SEQUENCE LISTING (80 sequences)

SEQ ID NO: 1 - protein, 116 aa, synthetic construct
  REGION 1..116: note = nanobody
QVQLVETGGGLVQAGGSLRLSCATSGTIFSRATMAWYRQTPGKQREWVTTITTSGNTNYADSVKGRFTISRDNAESTLYLQMNSLKPEDTAVYYCNAQFLSSRTNYWGKGTQVTVS

SEQ ID NO: 2 - protein, 122 aa, synthetic construct
  REGION 1..122: note = nanobody
QVQLVESGGGLVQAGGSLRLSCAASRRTFSGYAMGWFRQAPGKEREFVAAHWGSTSTHYADSVKGRFTISRDNAKNTVYLQMNTLKLEDTAVYYCAAKDRLGAVSDDQAYDYWGQGTQVTVS

SEQ ID NO: 3 - protein, 125 aa, synthetic construct
  REGION 1..125: note = Nanobody
QVQLVESGGGLVQAGGSLRLSCAASGRAFSSYPMGWFRQAPGKEREFLAAISGSGRLTYYADSVEGRFTISRDNAKNTVYLQMDVLTPEDTAIYYCAAQKPYASAQRWPPENYDFWGQGTQVTVS

SEQ ID NO: 4 - protein, 126 aa, synthetic construct
  REGION 1..126: note = Nanobody
QVQLVESGGGLVQAGGSLRLSCAASGRTFSRYAMGWFRQAPGKEREFVSAISGSGGFTDYADSVKGRFTISRDNAKSTVYLRMSSLKPEDTAVYYCAAEGSRGSSTRLDARGTYDYWGQGTQVTVS

SEQ ID NO: 5 - protein, 126 aa, synthetic construct
  REGION 1..126: note = Nanobody
QVQLVESGGGLVQAGGSLRLSCAASGRAFSSRPIGWFRQAPAKEREFVGVISQSGGFTDYEDSVKGRFTISRDNAKNTVYLQMSSLQPEDTAVYYCATGILFASGSAYYRPDDYDYWGQGTQVTVS

SEQ ID NO: 6 - protein, 125 aa, synthetic construct
  REGION 1..125: note = Nanobody
QVQLVETGGGLVQAGGSLRLSCAFSGRTFGNYAMAWFRQAPGKERDFVAYISWSGSSVFHADSVKGRFTISRDNAKNTVYLQMSSLKAEDTAVYYCAAAERTKRVSITDDTSYDYWGQGTQVTVS

SEQ ID NO: 7 - protein, 124 aa, synthetic construct
  REGION 1..124: note = Nanobody
QVQLVETGGGLVQAGGSLRLSCAASGRTFSSYEMAWFRQAPGKEREFVAVLSWTGRSTYYADSVKGRFTISRDNAKNTVYLQMNNLRPEDTAVYYCAADPPRHGYTWPPDGMDYWGKGTQVTVS

SEQ ID NO: 8 - protein, 120 aa, synthetic construct
  REGION 1..120: note = Nanobody
QVQLVESGGGFVQAGGSLRLSCVSSGGIFAIGKLAWYRQAPGQMREFVASISSGKIVSYESHVKGRFTISRDNTKDTVYLQMDSLKPEDTGVYYCAIQAPENWATTEYDYWGQGTQVTVS

SEQ ID NO: 9 - protein, 125 aa, synthetic construct
  REGION 1..125: note = Nanobody
QVQLVESGGGLVQAGGSLRLSCAASGRTGSSTTYRMAWFRQAPGKEREFVAHGTCNGGTYYADSVKGRFTISRNNAKNTVYLQMNNLKAEDTAVYYCAAARPTGASGRSVYDLDYWGQGTQVTVS

SEQ ID NO: 10 - protein, 124 aa, synthetic construct
  REGION 1..124: note = Nanobody
QVQLVETGGGLVQAGGSLRLSCAASGRTASDYVIAWFRQAPGKVRVFVAGISSGGTIKDYAESESVKGRFTISRDNAKNTVYLQMNSLKPEDTAVYYCAARLRVTDFDAASYDYWGQGTQVTVS

SEQ ID NO: 11 - protein, 122 aa, synthetic construct
  REGION 1..122: note = Nanobody
QVQLVESGGGLVQAGGSLRLSCAASSRTFSGYAVGWFRQAPRKEREFVAAFWGRTSTHYADSVKGRFTISRDNVKNTVYLQMNSLNPEDTAVYYCAAKDRLGAVSDDHAYDYWGQGTQVTVS

SEQ ID NO: 12 - protein, 124 aa, synthetic construct
  REGION 1..124: note = Nanobody
QVQLVESGGGLVQAGGSLRLSCAASGRTVSSYDMAWFRQAPGKERDFVAVISWTGRATYYEDSVKGRFTISRDDAKNTVYLQMNNLRPEDTAVYYCAADPPRHGASWPPSGMDYWGKGTQVTVS

SEQ ID NO: 13 - protein, 10 aa, synthetic construct
  REGION 1..10: note = CDR
GTIFSRATMA

SEQ ID NO: 14 - protein, 9 aa, synthetic construct
  REGION 1..9: note = CDR
TITTSGNTN

SEQ ID NO: 15 - protein, 10 aa, synthetic construct
  REGION 1..10: note = CDR
AQFLSSRTNY

SEQ ID NO: 16 - protein, 10 aa, synthetic construct
  REGION 1..10: note = CDR
RRTFSGYAMG

SEQ ID NO: 17 - protein, 9 aa, synthetic construct
  REGION 1..9: note = CDR
AHWGSTSTH

SEQ ID NO: 18 - protein, 16 aa, synthetic construct
  REGION 1..16: note = CDR
AKDRLGAVSDDQAYDY

SEQ ID NO: 19 - protein, 10 aa, synthetic construct
  REGION 1..10: note = CDR
GRAFSSYPMG

SEQ ID NO: 20 - protein, 10 aa, synthetic construct
  REGION 1..10: note = CDR
AISGSGRLTY

SEQ ID NO: 21 - protein, 18 aa, synthetic construct
  REGION 1..18: note = CDR
AQKPYASAQRWPPENYDF

SEQ ID NO: 22 - protein, 10 aa, synthetic construct
  REGION 1..10: note = CDR
GRTFSRYAMG

SEQ ID NO: 23 - protein, 10 aa, synthetic construct
  REGION 1..10: note = CDR
AISGSGGFTD

SEQ ID NO: 24 - protein, 19 aa, synthetic construct
  REGION 1..19: note = CDR
AEGSRGSSTRLDARGTYDY

SEQ ID NO: 25 - protein, 10 aa, synthetic construct
  REGION 1..10: note = CDR
GRAFSSRPIG

SEQ ID NO: 26 - protein, 10 aa, synthetic construct
  REGION 1..10: note = CDR
VISQSGGFTD

SEQ ID NO: 27 - protein, 19 aa, synthetic construct
  REGION 1..19: note = CDR
TGILFASGSAYYRPDDYDY

SEQ ID NO: 28 - protein, 10 aa, synthetic construct
  REGION 1..10: note = CDR
GRTFGNYAMA

SEQ ID NO: 29 - protein, 11 aa, synthetic construct
  REGION 1..11: note = CDR
AYISWSGSSVF

SEQ ID NO: 30 - protein, 18 aa, synthetic construct
  REGION 1..18: note = CDR
AAERTKRVSITDDTSYDY

SEQ ID NO: 31 - protein, 10 aa, synthetic construct
  REGION 1..10: note = CDR
GRTFSSYEMA

SEQ ID NO: 32 - protein, 10 aa, synthetic construct
  REGION 1..10: note = CDR
VLSWTGRSTY

SEQ ID NO: 33 - protein, 17 aa, synthetic construct
  REGION 1..17: note = CDR
ADPPRHGYTWPPDGMDY

SEQ ID NO: 34 - protein, 10 aa, synthetic construct
  REGION 1..10: note = CDR
GGIFAIGKLA

SEQ ID NO: 35 - protein, 10 aa, synthetic construct
  REGION 1..10: note = CDR
SISSGKIVSY

SEQ ID NO: 36 - protein, 14 aa, synthetic construct
  REGION 1..14: note = CDR
IQAPENWATTEYDY

SEQ ID NO: 37 - protein, 12 aa, synthetic construct
  REGION 1..12: note = CDR
GRTGSSTTYRMA

SEQ ID NO: 38 - protein, 9 aa, synthetic construct
  REGION 1..9: note = CDR
HGTCNGGTY

SEQ ID NO: 39 - protein, 17 aa, synthetic construct
  REGION 1..17: note = CDR
AARPTGASGRSVYDLDY

SEQ ID NO: 40 - protein, 10 aa, synthetic construct
  REGION 1..10: note = CDR
GRTASDYVIA

SEQ ID NO: 41 - protein, 10 aa, synthetic construct
  REGION 1..10: note = CDR
GISSGGTIKD

SEQ ID NO: 42 - protein, 15 aa, synthetic construct
  REGION 1..15: note = CDR
ARLRVTDFDAASYDY

SEQ ID NO: 43 - protein, 10 aa, synthetic construct
  REGION 1..10: note = CDR
SRTFSGYAVG

SEQ ID NO: 44 - protein, 9 aa, synthetic construct
  REGION 1..9: note = CDR
AFWGRTSTH

SEQ ID NO: 45 - protein, 16 aa, synthetic construct
  REGION 1..16: note = CDR
AKDRLGAVSDDHAYDY

SEQ ID NO: 46 - protein, 10 aa, synthetic construct
  REGION 1..10: note = CDR
GRTVSSYDMA

SEQ ID NO: 47 - protein, 10 aa, synthetic construct
  REGION 1..10: note = CDR
VISWTGRATY

SEQ ID NO: 48 - protein, 17 aa, synthetic construct
  REGION 1..17: note = CDR
ADPPRHGASWPPSGMDY

SEQ ID NO: 49 - protein, 132 aa, synthetic construct
  REGION 1..132: note = Nanobody
QVQLQESGGGLVPPGGSLRLSCAASGGPLDHYTIAWFRQAPGKEREVVSCIGSSDVSTYYADSVKGRFAISRDNVKNTVFLQMSSLKPEDTAVYLCAAKYVGSTSWGGGCAYELSVDRYLEVWGQGTQVSVS

SEQ ID NO: 50 - protein, 123 aa, synthetic construct
  REGION 1..123: note = Nanobody
QVQLQESGGGLVQAGGSLNLSCATSATFRVRLGPMSWYRQAPGKQRELVASITSGGSTNYSDSVRGRFTISRDNAKNAVYPQMNSLQTEDTAVYCCRADQDLPPGLIGTGAVVWGQGTQVAVS

SEQ ID NO: 51 - protein, 110 aa, synthetic construct
  REGION 1..110: note = Nanobody
EVQLQESGGGLVQPGGSLRLSCAASGTTINIGAARWYRQAPGKQREVVAGISFGGATNYIDSVKGRFTISRGDALNTWYLQMNNLKPEDTAVYYCNARGEWGQGTQVAVS

SEQ ID NO: 52 - protein, 114 aa, synthetic construct
  REGION 1..114: note = Nanobody
QVQLQESGGGLVQAGGSLRLSCVASGSGSSNNQVNWYRQAPGKQRELIARISTGGTTNYIDAVKGRFTIARDNAERSVYLQMNNLGPEDTAIYYCHAIIRARSYWGQGTLVTVS

SEQ ID NO: 53 - protein, 124 aa, synthetic construct
  REGION 1..124: note = Nanobody
EVQLQESGGGLVQAGGSLRLSCAASGDTICISAMGWYRQAPGKERELVAVITSADSTNYADSVKGRFTISRAGAKNTLYLQMNSLKPEDTAVYYCNAHTPKYSDFEVTCYEYDYWGQGTLVSVS

SEQ ID NO: 54 - protein, 10 aa, synthetic construct
  REGION 1..10: note = CDR
GGPLDHYTIA

SEQ ID NO: 55 - protein, 10 aa, synthetic construct
  REGION 1..10: note = CDR
CIGSSDVSTY

SEQ ID NO: 56 - protein, 25 aa, synthetic construct
  REGION 1..25: note = CDR
AKYVGSTSWGGGCAYELSVDRYLEV

SEQ ID NO: 57 - protein, 11 aa, synthetic construct
  REGION 1..11: note = CDR
ATFRVRLGPMS

SEQ ID NO: 58 - protein, 9 aa, synthetic construct
  REGION 1..9: note = CDR
SITSGGSTN

SEQ ID NO: 59 - protein, 16 aa, synthetic construct
  REGION 1..16: note = CDR
ADQDLPPGLIGTGAVV

SEQ ID NO: 60 - protein, 10 aa, synthetic construct
  REGION 1..10: note = CDR
GTTINIGAAR

SEQ ID NO: 61 - protein, 9 aa, synthetic construct
  REGION 1..9: note = CDR
GISFGGATN

SEQ ID NO: 62 - protein, 4 aa, synthetic construct
  REGION 1..4: note = CDR
ARGE

SEQ ID NO: 63 - protein, 10 aa, synthetic construct
  REGION 1..10: note = CDR
GSGSSNNQVN

SEQ ID NO: 64 - protein, 9 aa, synthetic construct
  REGION 1..9: note = CDR
RISTGGTTN

SEQ ID NO: 65 - protein, 8 aa, synthetic construct
  REGION 1..8: note = CDR
AIIRARSY

SEQ ID NO: 66 - protein, 10 aa, synthetic construct
  REGION 1..10: note = CDR
GDTICISAMG

SEQ ID NO: 67 - protein, 9 aa, synthetic construct
  REGION 1..9: note = CDR
VITSADSTN

SEQ ID NO: 68 - protein, 18 aa, synthetic construct
  REGION 1..18: note = CDR
AHTPKYSDFEVTCYEYDY

SEQ ID NO: 69 - protein, 167 aa, Apis mellifera
MQVVLGSLFLLLLSTSHGWQIRDRIGDNELEERIIYPGTLWCGHGNKSSGPNELGRFKHTDACCRTHDMCPDVMSAGESKHGLTNTASHTRLSCDCDDKFYDCLKNSADTISSYFVGKMYFNLIDTKCYKLEHPVTGCGERTEGRCLHYTVDKSKPKVYQWFDLRKY

SEQ ID NO: 70 - protein, 134 aa, Apis mellifera
IIYPGTLWCGHGNKSSGPNELGRFKHTDACCRTHDMCPDVMSAGESKHGLTNTASHTRLSCDCDDKFYDCLKNSADTISSYFVGKMYFNLIDTKCYKLEHPVTGCGERTEGRCLHYTVDKSKPKVYQWFDLRKY

SEQ ID NO: 71 - protein, 125 aa, synthetic construct
QVQLVESGGGLVQAGGSLRLSCAASGFTLDYYAIGWFRQAPGKEREGVSCITSYDDSTYYTDSVKGRFTISRDNAKNTVYLQMNSLKPEDTAVYYCAAVGTWYLVNWWAPSEYDYWGQGTQVTVS

SEQ ID NO: 72 - protein, 113 aa, synthetic construct
QVQLVESGGGLVQAGGSLRLSCAASGSIFNWAWYRQFPGKQREWVATITSGGNTNLADSVKGRFTISLDNAKNTMYLQMNSLKPEDTAVYYCNSARFRNRDAIWGQGTQVTVS

SEQ ID NO: 73 - protein, 10 aa, synthetic construct
GFTLDYYAIG

SEQ ID NO: 74 - protein, 10 aa, synthetic construct
CITSYDDSTY

SEQ ID NO: 75 - protein, 18 aa, synthetic construct
AVGTWYLVNWWAPSEYDY

SEQ ID NO: 76 - protein, 7 aa, synthetic construct
GSIFNWA

SEQ ID NO: 77 - protein, 9 aa, synthetic construct
TITSGGNTN

SEQ ID NO: 78 - protein, 10 aa, synthetic construct
SARFRNRDAI

SEQ ID NO: 79 - DNA, 375 bp, synthetic construct
  source 1..375: mol_type = other DNA
caggtgcagctcgtggagtcagggggaggcctggtgcaggctggggggtctctgagactctcctgtgcagcctctggattcactttggattattatgccataggctggttccgccaggccccagggaaggagcgcgagggggtctcatgtattactagttatgatgatagcacatactatacagactccgtgaagggccgattcaccatctccagagacaatgccaagaacacggtgtatctgcaaatgaacagcctgaaacctgaggacacggccgtttattactgtgccgcagtgggtacctggtatctcgtaaactggtgggccccatctgagtatgactactggggccaggggacccaggtcaccgtctcc

SEQ ID NO: 80 - DNA, 339 bp, synthetic construct
  source 1..339: mol_type = other DNA
caggtgcagctcgtggagtcggggggaggcttggtgcaggctggggggtctctgagactctcctgtgcagcctctggaagcatcttcaattgggcctggtaccgccagtttccagggaaacagcgcgagtgggttgcgacgattactagtggtggtaacacaaacttggcagactccgtgaagggccgattcaccatctccttggacaacgccaagaacacgatgtatctgcagatgaacagcctgaaacctgaagacacagccgtctattattgtaattccgcccggttccgcaacagagatgccatctggggccaggggacccaggtcaccgtctcc

(19) United States
(12) Patent Application Publication
(10) Pub. No.: US 2025/0206812 A1
(43) Pub. Date: Jun. 26, 2025

(54) SINGLE DOMAIN ANTIBODIES AGAINST BEE VENOM EPITOPES

(71) Applicant: AARHUS UNIVERSITET, Aarhus C (DK)

(72) Inventors: Edzard SPILLNER, Aarhus C (DK); Josephine Baunvig AAGAARD, Aarhus C (DK); Coline SIVELLE, Aarhus C (DK); Michaela Yvonne MIEHE, Aarhus C (DK)

(21) Appl. No.: 18/844,120

(22) PCT Filed: Mar. 10, 2023
(86) PCT No.: PCT/EP2023/056199

§ 371 Date: Sep. 5, 2024

(30) Foreign Application Priority Data
Mar. 11, 2022 (EP) 22161556.0
Aug. 16, 2022 (EP) 22190607.6

(51) Int. Cl. C07K 16/18; C07K 16/40

(52) U.S. Cl. CPC C07K 16/18; C07K 16/40; C12Y 301/01004; C07K 2317/24; C07K 2317/31; C07K 2317/569; C07K 2317/76; C07K 2317/92

(57) ABSTRACT

The present invention relates to single domain antibodies and multispecific antibodies that a) bind to the allergen from honeybee venom Api m 1 or Api m 2, b) exhibit non-overlapping epitopes with other antibodies, c) exhibit favourable kinetic properties; and/or d) have the ability to block the binding of IgE to the allergen. The present invention also relates to uses of these single domain antibodies and multispecific antibodies, in particular in the treatment or prevention of bee allergy. FIG. 1B to be published with the abstract.

### TECHNICAL FIELD OF THE INVENTION

The present invention relates to antibodies, such as single domain antibodies, against bee venom epitopes, such as honeybee venom epitopes. In particular, the present invention relates to the use of such antibodies alone or in combination for passive immunisation as medicaments, preferably for seasonal protection and/or treatment of bee allergy.

### BACKGROUND OF THE INVENTION

Central to immunity is antibody (Ab) recognition of antigen(s)—Ag(s). There are five different Ab classes: IgG, IgM, IgE, IgD, IgA. Two Ab Fab fragments connect to the Fc fragment and two Ag molecules are recognized by the Fabs. The IgE isotype is crucial for allergic diseases and acts as a key molecule in a network of proteins, including the high-affinity IgE receptor FcεRI, the low-affinity receptor CD23, and galectins, e.g., galectin-32. Upon crosslinking by allergens, IgE bound to FcεRI on mast cells and basophils triggers degranulation, release of proinflammatory mediators, and immediate reactions.

Hymenoptera venom allergy (HVA) is one of the most common causes of anaphylaxis in adults and frequently associated with severe anaphylaxis. It results in significant morbidity and impairment in quality of life. A prevalence of up to 3.5% is reported in Europe. The risk of a systemic reaction in sensitized subjects with no previous history of HVA however is between 3.3 and 5%. In Central and Northern Europe, the main perpetrators of HVA are honeybees (Apis) and yellow jackets (Vespula).

Treatment of allergic diseases can be pursued in different ways. Vaccination against the allergens is typically done by injecting increasing doses of allergens resulting in the establishment of an IgG response in the patient that interferes with the allergic trigger by blocking the interaction of the allergen and the IgE on the surface of effector cells.

The IgE response in venom allergic patients is typically directed to a set of major and minor allergens. The best-characterised and most abundant HBV components are phospholipase A2 (Api m 1), the venom hyaluronidase (Api m 2) and the basic peptide melittin (Api m 4). Api m 1 encodes a signal peptide of 18 amino acids, a propeptide of 15 residues, and a mature peptide of 134 residues. The catalytic activity and crystal structure are well described and it contains 5 disulfide bonds and an oligosaccharide attached to asparagine 13. Api m 2 is a polypeptide of 350 residues specifically cleaving the β-1,4 glycosidic bond between GlcNAc and GlcA of long hyaluronic acid chains. The protein contains 2 disulfide bonds and 4 potential glycosylation sites. Additional HBV allergens of lower abundance have been characterized such as the acid phosphatase (Api m 3), dipeptidylpeptidase IV (Api m 5), and icarapin (Api m 10).

Pucca et al. review knowledge on bee venom and bee envenoming therapy (Bee Updated: Current Knowledge on Bee Venom and Bee Envenoming Therapy. Front Immunol. 2019 Sep. 6; 10:2090).

Funayama et al. disclose the production of human antibody fragments binding to melittin and phospholipase A2 in Africanised Bee Venom (Production of Human Antibody Fragments Binding to Melittin and Phospholipase A2 in Africanised Bee Venom: Minimising Venom Toxicity. Basic & Clinical Pharmacology & Toxicology, 2012, 110, 290-297).

Pessenda et al. disclose the identification of phages specific to PLA2 from Africanized Apis mellifera bees, also known as killer bees. Specific clones for melittin and PLA2 were selected for the production of soluble scFvs, named Afribumabs (Human scFv antibodies (Afribumabs) against Africanized bee venom: Advances in melittin recognition. Toxicon. 2016 Mar. 15; 112:59-67).

Schneider et al. disclose human monoclonal antibodies against epitopes on bee venom phospholipase A2 (PLA2) (Human monoclonal or polyclonal antibodies recognize predominantly discontinuous epitopes on bee venom phospholipase A₂. J ALLERGY CLIN IMMUNOL. Vol 94. Pages 61-70).

Hence, an improved method to treat or prevent allergies would be advantageous, and in particular a more efficient and/or reliable medical composition for use in the treatment or prevention of allergies would be advantageous.

### SUMMARY OF THE INVENTION

The present invention relates to passive immunisation by administering allergen-specific antibodies/nanobodies for (seasonal) protection and treatment of allergies, in particular bee allergy. More specifically, the present invention relates to the targeted identification of single domain antibody candidates that
a) bind to the allergen from honeybee venom Api m 1 or Api m 2;
b) exhibit non-overlapping epitopes with other candidates;
c) exhibit favourable kinetic properties; and/or
d) have the ability to block the binding of IgE to the allergen.

Examples 1 and 2 show production and identification of such Api m 1 and Api m 2 single domain antibodies.

Example 3 shows that the identified single domain antibody AM1-1 binds a different epitope on Api m 1 than the other nanobodies.

Examples 4-5 show generation and characterization of Nanobody-hIgE formats. Nanobodies mimicking human IgE may be interesting for diagnostic and standardisation purposes. Importantly, this characterization also identifies Api m 1 and Api m 2 targeting nanobodies with non-overlapping epitopes.

Example 6 shows generation and characterization of bi-specific single domain antibodies-based IgGs that bind two different epitopes at the same time potentially increasing inhibitory potential.

Example 7 shows that the nanobodies according to the invention inhibit the binding of patient IgE to immobilized allergens.

Example 8 shows generation and characterization of mono- and bi-specific single domain antibody-based IgGs that bind two different epitopes at the same time potentially increasing inhibitory potential.

Example 9 shows binding kinetics of nanobody-based Human IgG1 formats according to the invention.

Example 10 further shows that the nanobodies according to the invention inhibit the binding of patient IgE to immobilized allergens.

Example 11 shows a crystal structure of Api m 1 in complex with the nanobodies AM1-1 and AM1-4 with a resolution of 1.8 Å. The structure shows that the two nanobodies binds to distinct epitopes on opposite sides of the Api m 1 molecule and that the nanobody AM1-1 is interacting directly with the active site of Api m1.

Example 12 shows that the nanobody AM1-1 can reduce the enzymatic activity of Api m 1 to 50%. Thus, in addition to blocking IgE binding to the allergen, the nanobody AM1-1 might therefore also reduce reactions associated with the enzymatic activity of Api m 1, such as venom induced toxicity.

Example 13 shows a clear potential in using nanobody-based formats according to the invention for inhibiting basophil activation in honeybee venom allergic patients. This effect was observed for single nanobody-based hIgGs, however, when combining the nanobody-based hIgG either as a mix or as a bispecific molecule, an additive or slightly synergetic effect was observed.

Example 14 shows a nanobody phage library derived from an Api m 1 immunized llama re-selected in order to identify nanobodies binding a distinct epitope than the nanobody AM1-4. In this selection the inventors selected nanobodies against immobilized AM1-4 IgG/Api m 1 complexes and identified two new Api m 1 specific nanobodies (AM1-21 and AM1-22).

Example 15 shows that the inventors successfully generated nanobody-based human IgG₁ formats comprising the nanobodies AM1-21 and AM1-22 which were found in Example 14.

Example 16 shows that the nanobody AM1-21 binds an epitope that does not overlap with AM1-1 or AM1-22. However, the nanobody-based IgG1 formats cannot bind at the same time suggesting steric hindrance by the chosen larger format.

Example 17 shows that the nanobody-based human IgG1 formats AM1-21 IgG1 and AM1-22 IgG1 bind Api m 1 with affinities in the picomolar range. Said affinities are approximately 100-fold higher than that of the AM1-1 IgG1. The nanobodies with strongly increased affinities and the epitopes in proximity or similar to the one of AM1-1 could potentially provide an advantage when applied for blocking purposes.

In sum, the above described single domain antibodies and bispecific “single domain antibody”-based IgG's may be used in the passive immunisation against bee venom allergy or for enzymatic inhibition.

Thus, an object of the present invention relates to the provision of compounds and composition for improving treatment, prevention and/or alleviation of bee venom allergy.

Another object of the present invention relates to the provision of compounds and composition for improving treatment, prevention and/or alleviation of a venom induced toxicity.

In particular, it is an object of the present invention to provide single domain antibodies targeting different epitopes on bee venom allergens, such as honeybee venom allergens.

Thus, one aspect of the invention relates to a single domain antibody having binding affinity for Api m 1 (PLA2), wherein said single domain antibody comprises complementarity determining region (CDR) domains selected from the group consisting of:
i. CDR domains:
   CDR1 according to SEQ ID NO: 13;
   CDR2 according to SEQ ID NO: 14; and
   CDR3 according to SEQ ID NO: 15;
ii. CDR domains:
   CDR1 according to SEQ ID NO: 16;
   CDR2 according to SEQ ID NO: 17; and
   CDR3 according to SEQ ID NO: 18;
iii. CDR domains:
   CDR1 according to SEQ ID NO: 19;
   CDR2 according to SEQ ID NO: 20; and
   CDR3 according to SEQ ID NO: 21;
iv. CDR domains:
   CDR1 according to SEQ ID NO: 22;
   CDR2 according to SEQ ID NO: 23; and
   CDR3 according to SEQ ID NO: 24;
v. CDR domains:
   CDR1 according to SEQ ID NO: 25;
   CDR2 according to SEQ ID NO: 26; and
   CDR3 according to SEQ ID NO: 27;
vi. CDR domains:
   CDR1 according to SEQ ID NO: 28;
   CDR2 according to SEQ ID NO: 29; and
   CDR3 according to SEQ ID NO: 30;
vii. CDR domains:
   CDR1 according to SEQ ID NO: 31;
   CDR2 according to SEQ ID NO: 32; and
   CDR3 according to SEQ ID NO: 33.
viii. CDR domains:
   CDR1 according to SEQ ID NO: 34;
   CDR2 according to SEQ ID NO: 35; and
   CDR3 according to SEQ ID NO: 36;
ix. CDR domains:
   CDR1 according to SEQ ID NO: 37;
   CDR2 according to SEQ ID NO: 38; and
   CDR3 according to SEQ ID NO: 39;
x. CDR domains:
   CDR1 according to SEQ ID NO: 40;
   CDR2 according to SEQ ID NO: 41; and
   CDR3 according to SEQ ID NO: 42;
xi. CDR domains:
   CDR1 according to SEQ ID NO: 43;
   CDR2 according to SEQ ID NO: 44; and
   CDR3 according to SEQ ID NO: 45;
xii. CDR domains:
   CDR1 according to SEQ ID NO: 46;
   CDR2 according to SEQ ID NO: 47; and
   CDR3 according to SEQ ID NO: 48;
xiii. CDR domains:
   CDR1 according to SEQ ID NO: 73;
   CDR2 according to SEQ ID NO: 74; and
   CDR3 according to SEQ ID NO: 75;
xiv. CDR domains:
   CDR1 according to SEQ ID NO: 76;
   CDR2 according to SEQ ID NO: 77; and
   CDR3 according to SEQ ID NO: 78;
or single domain antibodies according to any of i.-xii. having 1-3 substitutions in one or more of the CDR1, CDR2 and CDR3 domains, preferably 1-2 substitutions and more preferably 1 substitution in one or more of the CDR1, CDR2 and CDR3 domains;

### OR

a single domain antibody having binding affinity for Api m 2, wherein said single domain antibody comprises CDR domains selected from the group consisting of:
xv. CDR domains:
   CDR1 according to SEQ ID NO: 54;
   CDR2 according to SEQ ID NO: 55; and
   CDR3 according to SEQ ID NO: 56;
xvi. CDR domains:
   CDR1 according to SEQ ID NO: 57;
   CDR2 according to SEQ ID NO: 58; and
   CDR3 according to SEQ ID NO: 59;
xvii. CDR domains:
   CDR1 according to SEQ ID NO: 60;
   CDR2 according to SEQ ID NO: 61; and
   CDR3 according to SEQ ID NO: 62;
xviii. CDR domains:
   CDR1 according to SEQ ID NO: 63;
   CDR2 according to SEQ ID NO: 64; and
   CDR3 according to SEQ ID NO: 65;
xix. CDR domains:
   CDR1 according to SEQ ID NO: 66;
   CDR2 according to SEQ ID NO: 67; and
   CDR3 according to SEQ ID NO: 68;
or single domain antibodies according to any of xiii.-xvii. having 1-3 substitutions in one or more of the CDR1, CDR2 and CDR3 domains, preferably 1-2 substitutions and more preferably 1 substitution in one or more of the CDR1, CDR2 and CDR3 domains.

In a preferred embodiment, the invention relates to a single domain antibody, wherein said single domain antibody comprises CDR domains selected from the group consisting of:
i. CDR domains:
   CDR1 according to SEQ ID NO: 13;
   CDR2 according to SEQ ID NO: 14; and
   CDR3 according to SEQ ID NO: 15;
ii. CDR domains:
   CDR1 according to SEQ ID NO: 16;
   CDR2 according to SEQ ID NO: 17; and
   CDR3 according to SEQ ID NO: 18;
iii. CDR domains:
   CDR1 according to SEQ ID NO: 19;
   CDR2 according to SEQ ID NO: 20; and
   CDR3 according to SEQ ID NO: 21;
iv. CDR domains:
   CDR1 according to SEQ ID NO: 22;
   CDR2 according to SEQ ID NO: 23; and
   CDR3 according to SEQ ID NO: 24;
v. CDR domains:
   CDR1 according to SEQ ID NO: 25;
   CDR2 according to SEQ ID NO: 26; and
   CDR3 according to SEQ ID NO: 27;
vi. CDR domains:
   CDR1 according to SEQ ID NO: 28;
   CDR2 according to SEQ ID NO: 29; and
   CDR3 according to SEQ ID NO: 30;
vii. CDR domains:
   CDR1 according to SEQ ID NO: 31;
   CDR2 according to SEQ ID NO: 32; and
   CDR3 according to SEQ ID NO: 33.
viii. CDR domains:
   CDR1 according to SEQ ID NO: 34;
   CDR2 according to SEQ ID NO: 35; and
   CDR3 according to SEQ ID NO: 36;
ix. CDR domains:
   CDR1 according to SEQ ID NO: 37;
   CDR2 according to SEQ ID NO: 38; and
   CDR3 according to SEQ ID NO: 39;
x. CDR domains:
   CDR1 according to SEQ ID NO: 40;
   CDR2 according to SEQ ID NO: 41; and
   CDR3 according to SEQ ID NO: 42;
xi. CDR domains:
   CDR1 according to SEQ ID NO: 43;
   CDR2 according to SEQ ID NO: 44; and
   CDR3 according to SEQ ID NO: 45;
xii. CDR domains:
   CDR1 according to SEQ ID NO: 46;
   CDR2 according to SEQ ID NO: 47; and
   CDR3 according to SEQ ID NO: 48.
xiii. CDR domains:
   CDR1 according to SEQ ID NO: 73;
   CDR2 according to SEQ ID NO: 74; and
   CDR3 according to SEQ ID NO: 75;
      and
xiv. CDR domains and:
   CDR1 according to SEQ ID NO: 76;
   CDR2 according to SEQ ID NO: 77; and
   CDR3 according to SEQ ID NO: 78;

### OR

a single domain antibody having binding affinity for Api m 2, wherein said single domain antibody comprises CDR domains selected from the group consisting of:
xv. CDR domains:
   CDR1 according to SEQ ID NO: 54;
   CDR2 according to SEQ ID NO: 55; and
   CDR3 according to SEQ ID NO: 56;
xvi. CDR domains:
   CDR1 according to SEQ ID NO: 57;
   CDR2 according to SEQ ID NO: 58; and
   CDR3 according to SEQ ID NO: 59;
xvii. CDR domains:
   CDR1 according to SEQ ID NO: 60;
   CDR2 according to SEQ ID NO: 61; and
   CDR3 according to SEQ ID NO: 62;
xviii. CDR domains:
   CDR1 according to SEQ ID NO: 63;
   CDR2 according to SEQ ID NO: 64; and
   CDR3 according to SEQ ID NO: 65;
xix. CDR domains:
   CDR1 according to SEQ ID NO: 66;
   CDR2 according to SEQ ID NO: 67; and
   CDR3 according to SEQ ID NO: 68.

In an even more preferred embodiment, the invention relates to a single domain antibody having binding affinity for Api m 1 (PLA2), wherein said single domain antibody comprises the CDR domains (the AM1-1 antibody):
CDR1 according to SEQ ID NO: 13;
CDR2 according to SEQ ID NO: 14; and
CDR3 according to SEQ ID NO: 15.

As shown in example 3, such single domain antibody (annotated AM1-1 in here) binds to an epitope on Api m 1 different from the other identified nanobodies targeting Api m 1. As shown in example 11, this epitope is at the active site of Api m 1.

Another aspect of the present invention relates to a multi-specific antibody, such as a bispecific antibody, comprising at least one of the single domain antibodies according to the invention. Bispecific antibodies are characterized in example 6.

Yet another aspect of the present invention is to provide a composition or combination comprising one or more of the single domain antibodies according to the invention and/or a multi-specific antibody according to the invention.

A further aspect of the invention relates to the single domain antibody according to the invention, the multi-specific antibody according to the invention or the composition or combination according to the invention, for use as a medicament. Example 7 and 10 shows that the nanobodies according to the invention inhibit the binding of patient IgE to immobilized allergens.

Still a further aspect of the invention relates to the single domain antibody according to the invention, the multi-specific antibody according to the invention or the composition or combination according to the invention, for use in the treatment, prevention and/or alleviation of bee venom allergy, such as bee venom allergy caused by the honeybee (Apis) venom.

Another aspect of the invention relates to the single domain antibody according to the invention, the multi-specific antibody according to the invention or the composition or combination according to the invention, for use in providing passive immunity against bee venom allergy.

Still another aspect of the invention relates to the single domain antibodies according to the invention, the multispecific antibodies according to the invention and/or the composition or combination according to the invention for use in the treatment, prevention and/or alleviation of a venom induced toxicity.

Yet an aspect of the invention relates to
a) a nucleic acid encoding a single domain antibody according to the invention or encoding a multispecific antibody according to the invention; and/or
b) a vector comprising the nucleic acid according to a), such as an expression vector, such as a plasmid; and/or
c) a host cell comprising the vector according to b).

Still another aspect of the present invention is to provide in vitro use of a single domain antibody according to the invention, a multi-specific antibody according to the invention or a composition or combination according to the invention, such as for standardization, such as for standardizations of diagnostic or therapeutic products, such as honeybee venom products.

A further aspect of the invention is a single domain antibody or multi-specific antibody, such as a bispecific antibody having binding affinity for the active site of Api m 1.

### BRIEF DESCRIPTION OF THE FIGURES

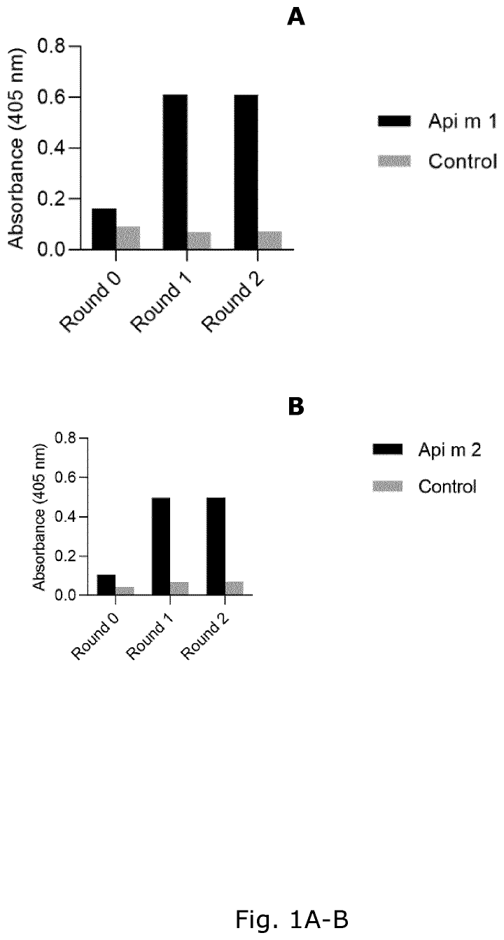

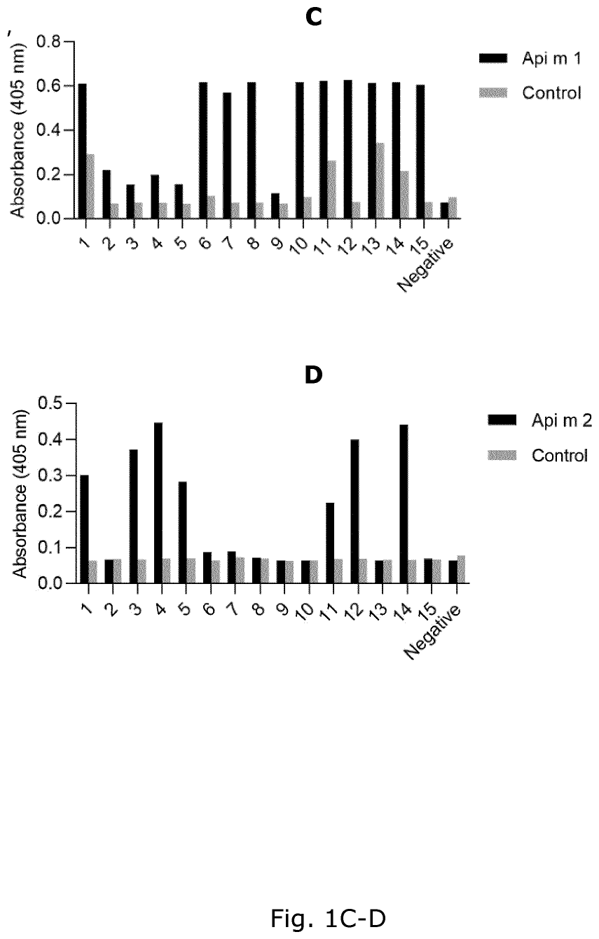

### FIG. 1

FIG. 1 shows the enrichment of the libraries. (A-B) assessed in ELISA by monitoring the immunoreactivity of polyclonal phages. (C-D) Binding of phages to immobilized allergens was detected using anti-M13 antibody conjugated to horseradish peroxidase. Immunoreactivity of monoclonal phages against Api m 1 or Api m 2 was analyzed in ELISA as above.

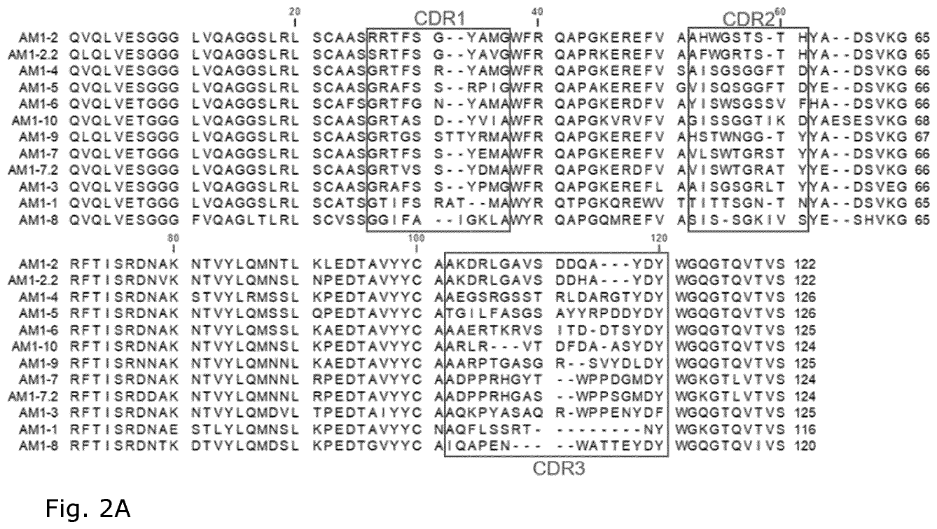

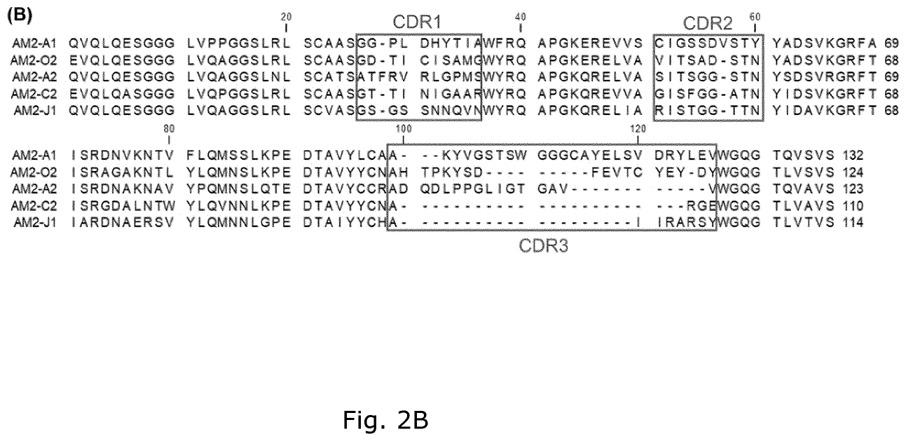

### FIG. 2

FIG. 2 shows sequence alignment of the nanobodies obtained by selection against (A) Api m 1 and (B) Api m 2. The Complementarity-determining regions (CDRs) are boxed. The individual full-length sequences and individual CDR domains are listed in Tables 3-6 including their unique names and sequence identifiers (SEQ ID's).

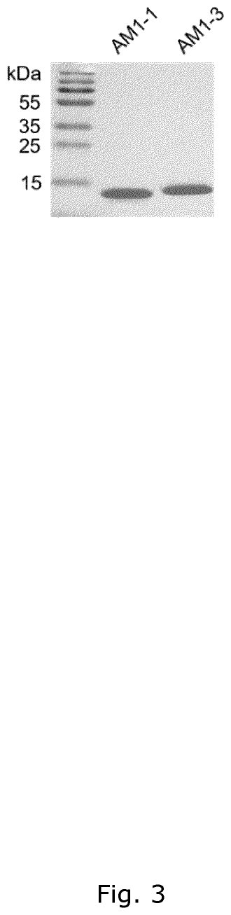

### FIG. 3

FIG. 3 shows expressed nanobodies AM1-1 and AM1-3, purified and assessed by SDS-PAGE.

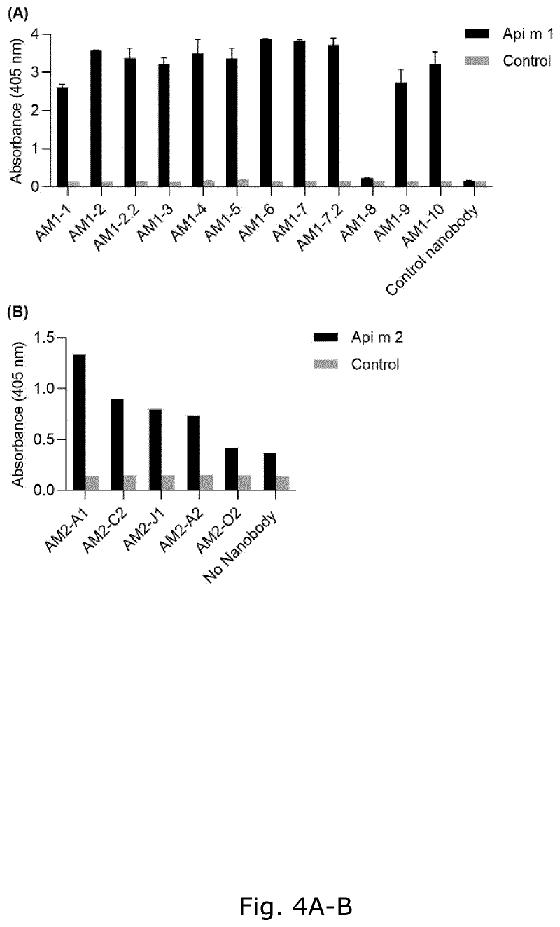

### FIG. 4

FIG. 4 shows the immunoreactivity of purified nanobodies assessed in ELISA. (A) Binding to immobilized Api m 1 was detected using anti-V5 antibody. Data are mean±s.d. of duplicates. (B) Binding to immobilized rApi m 2 was detected using mouse anti-penta-His and AP-conjugated rabbit anti-mouse IgG antibody.

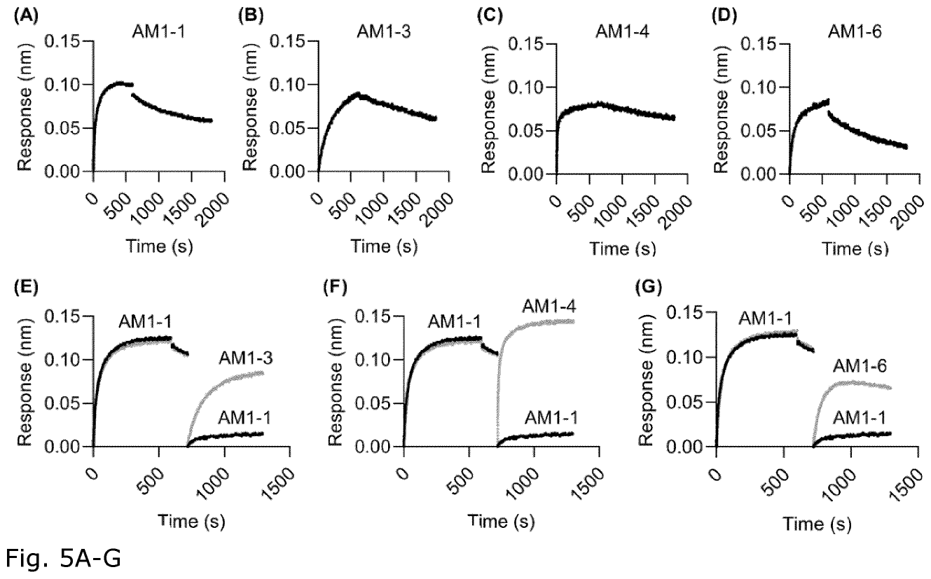

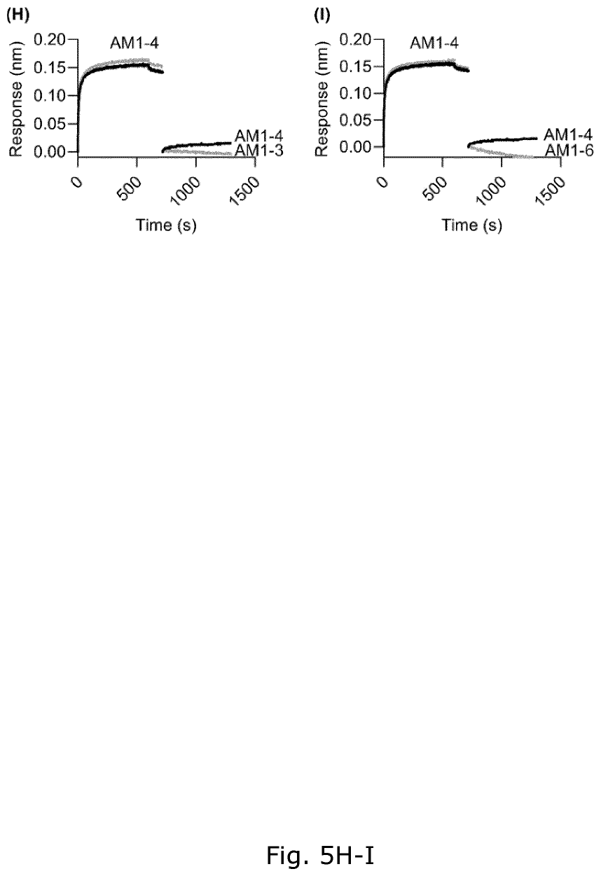

### FIG. 5

FIG. 5 shows BLI analysis of the nanobodies binding to Api m 1 immobilized on amino reactive biosensors. (A-D) Nanobody association was measured for 600 seconds followed by 1200 seconds of dissociation. (E-I) For epitope binding, nanobody association was followed by a short dissociation and association of a second nanobody.

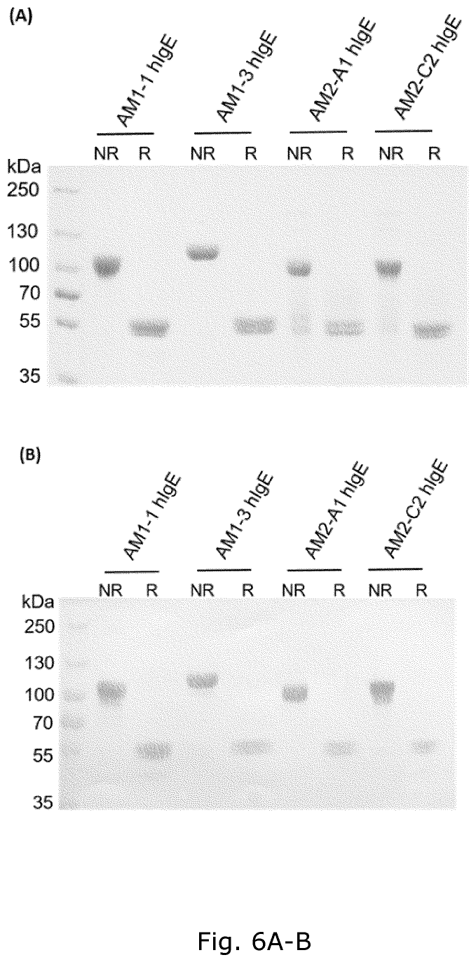

### FIG. 6

FIG. 6 shows (A) SDS-PAGE and (B) immunoblotting analysis of Nb-IgEs under reducing (R) and non-reducing (NR) conditions. The immunoblot was developed using anti-IgE antibody conjugated to alkaline phosphatase.

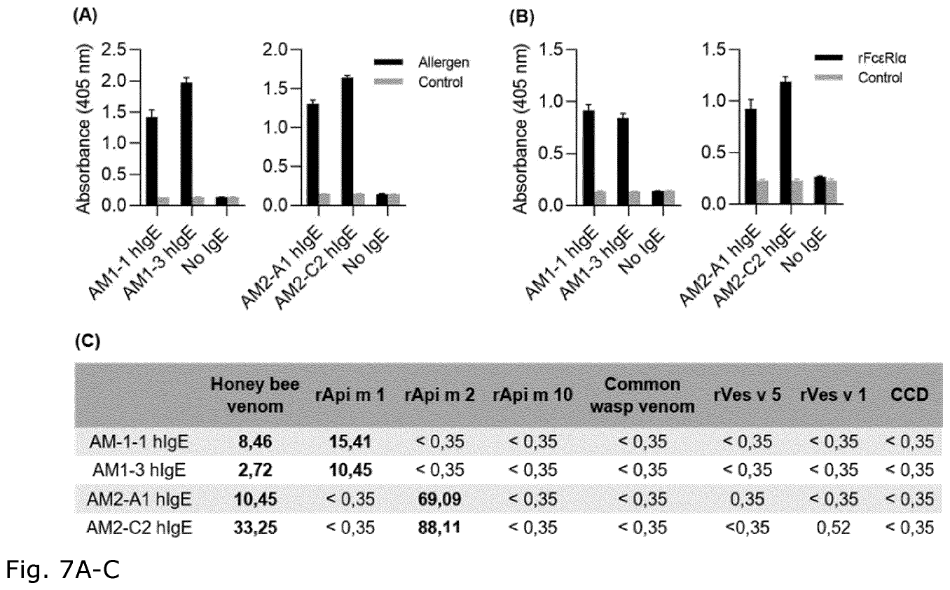

### FIG. 7

FIG. 7 shows the immunoreactivity of purified nb-IgEs assessed in ELISA. (A) Binding to immobilized allergens or (B) FcεRIα was detected using anti-IgE antibody conjugated to alkaline phosphatase. Data are mean±s.d. of triplicates. (C) The immunoreactivity of purified nb-IgE was further assessed in a commercial test platform, a Euroline assay, according to the manufacturer's instruction.

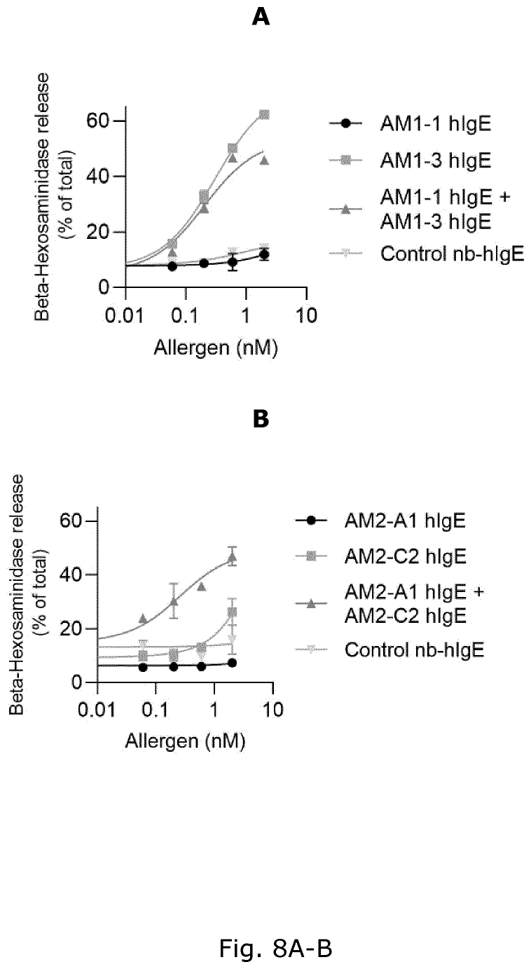

### FIG. 8

FIG. 8A-B shows RBL-SX38 cells expressing the human FcεRIα sensitized with single Nb-IgEs or pairs of Nb-IgEs. Degranulation was induced by series dilutions of allergens and monitored by released Beta-Hexosaminidase activity. Data are mean±s.d. of duplicates. (A) AM1-1 hIgE and AM1-3 hIgE. (B) AM2-A1 hIgE and AM2-C2 hIgE.

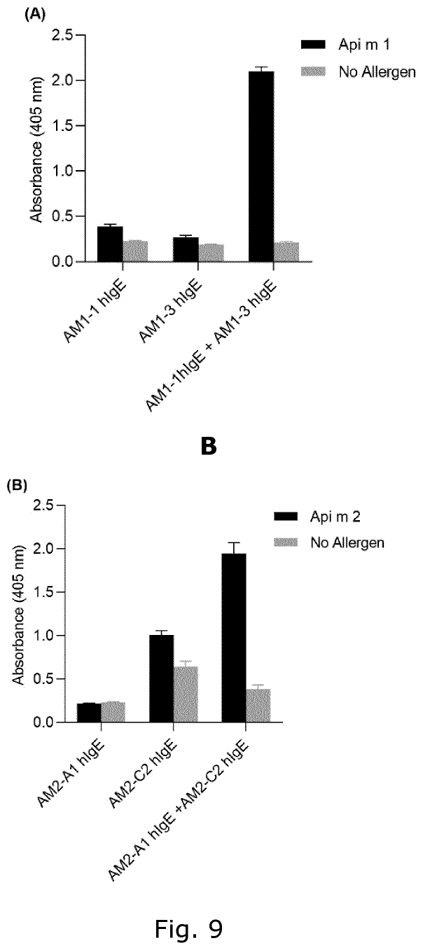

### FIG. 9

FIG. 9 shows Nb-IgEs pre-incubated with allergens. Binding of the complexes to immobilized CD23 was detected using biotin conjugated anti-IgE antibody. Data are mean±s.d. of duplicates or triplicates. (A) AM1-1 hIgE and AM1-3 hIgE. (B) AM2-A1 hIgE and AM2-C2 hIgE.

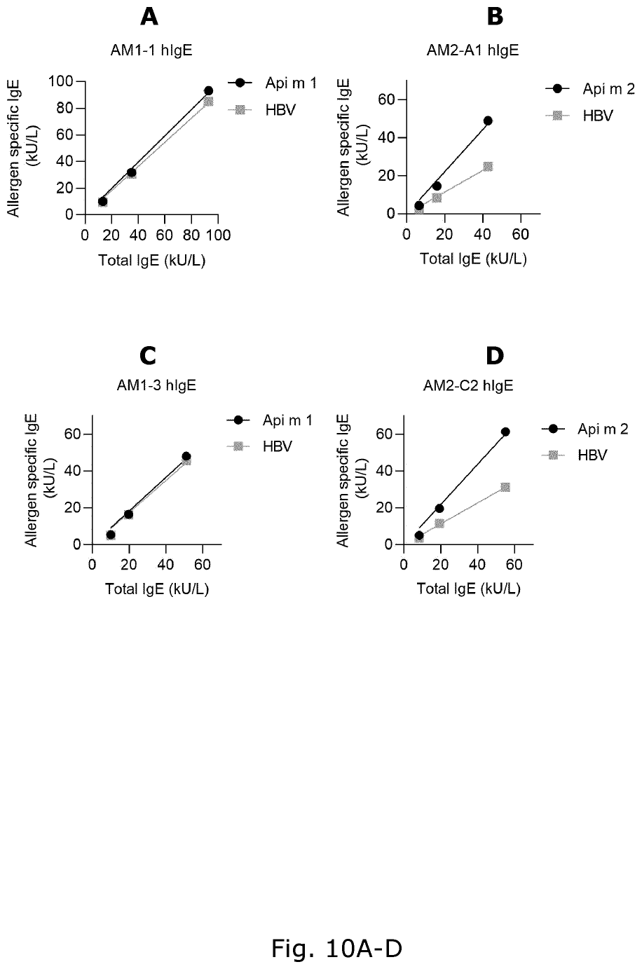

### FIG. 10

FIG. 10 shows total IgE and allergen specific IgE measured on dilution series of the nb-IgEs using immunoCAPs. (A) AM1-1 hIgE; (B) AM2-A1 hIgE. (C) AM1-3 hIgE. (D) AM2-C2 hIgE.

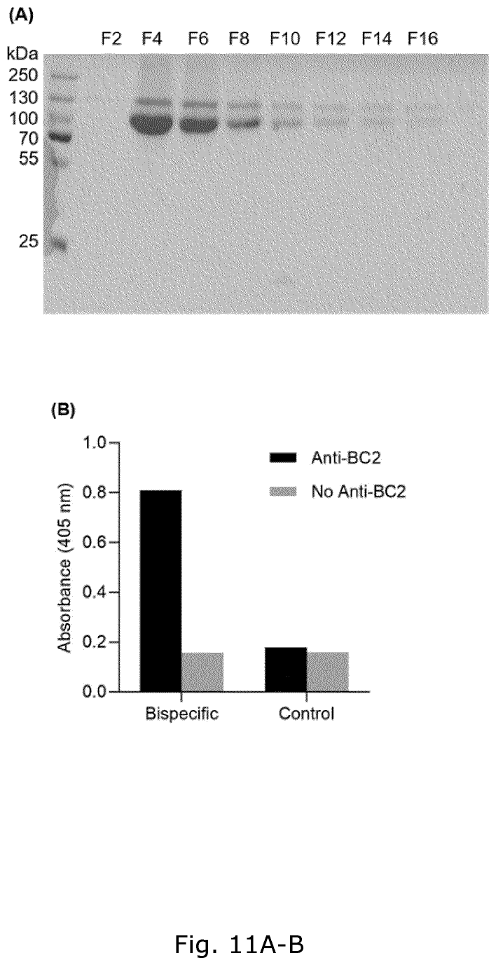

### FIG. 11

FIG. 11 shows (A) expressed bispecific nanobody-based IgG1 purified by a polyhistidine-tag and (B) the elution fractions (F2 to F14) were assessed by SDS-PAGE. Heterodimerization of the antibodies was confirmed by ELISA. Immobilized bispecific antibody was detected using biotinylated anti-BC2 antibody.

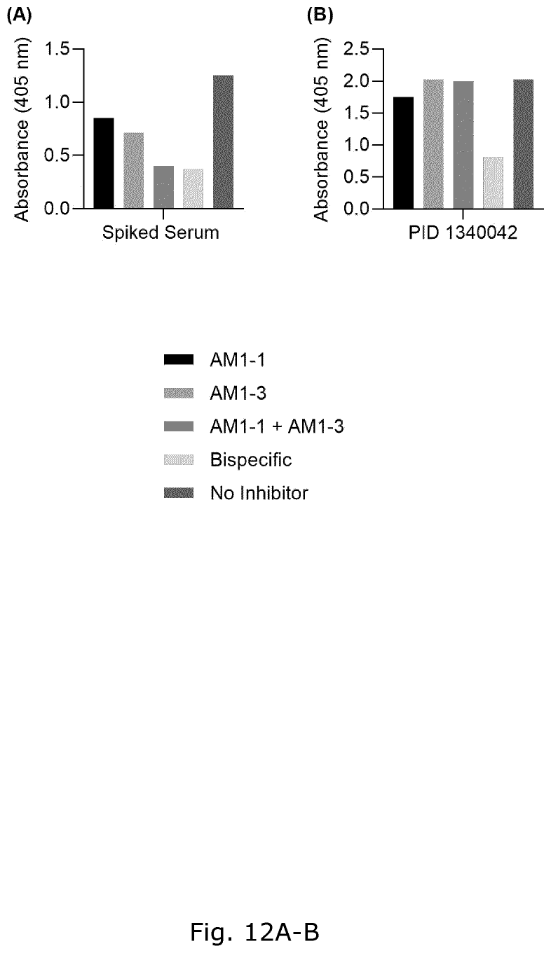

### FIG. 12

FIG. 12 shows inhibition of IgE binding to immobilized Api m 1 tested by incubation with different nanobodies and nanobody-formats. Bound IgE was detected using anti-IgE antibody conjugated to alkaline phosphatase. (A) Negative serum spiked with nb-hIgEs was used as a positive control of the assay. (B) Inhibition of bee venom allergic patient serum containing Api m 1 specific IgEs was assessed.

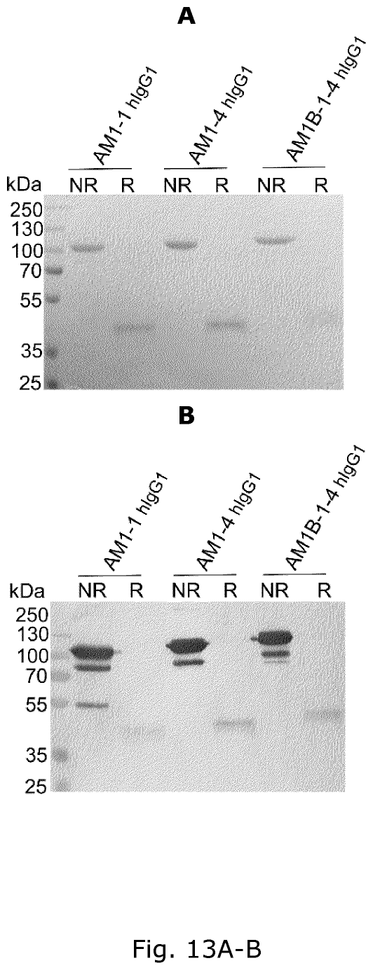

### FIG. 13

FIG. 13 shows SDS-PAGE (A) and immunoblotting (B) analysis of nanobody-based human IgG1 formats under reducing (R) and non-reducing (NR) conditions. The immunoblot was developed using anti-human IgG antibody conjugated to alkaline phosphatase.

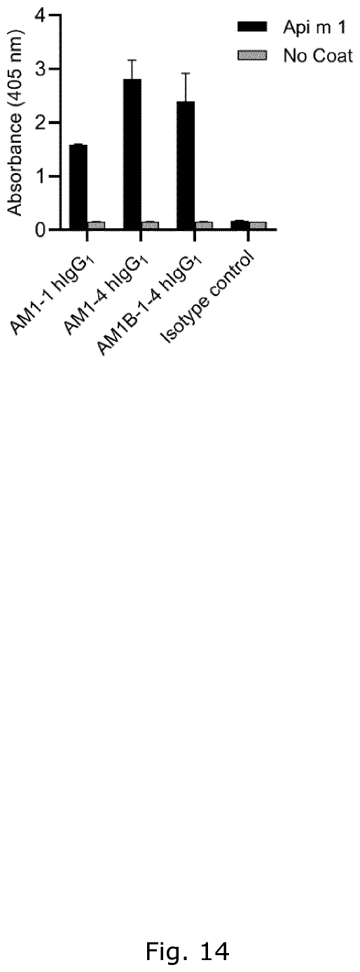

### FIG. 14

FIG. 14 shows immunoreactivity of purified nanobody-based human IgG1 assessed by ELISA. Nanobody-based human IgG₁ bound to immobilized Api m 1 was detected using anti-human IgG antibody conjugated to alkaline phosphatase. Data are mean±s.d. of triplicates.

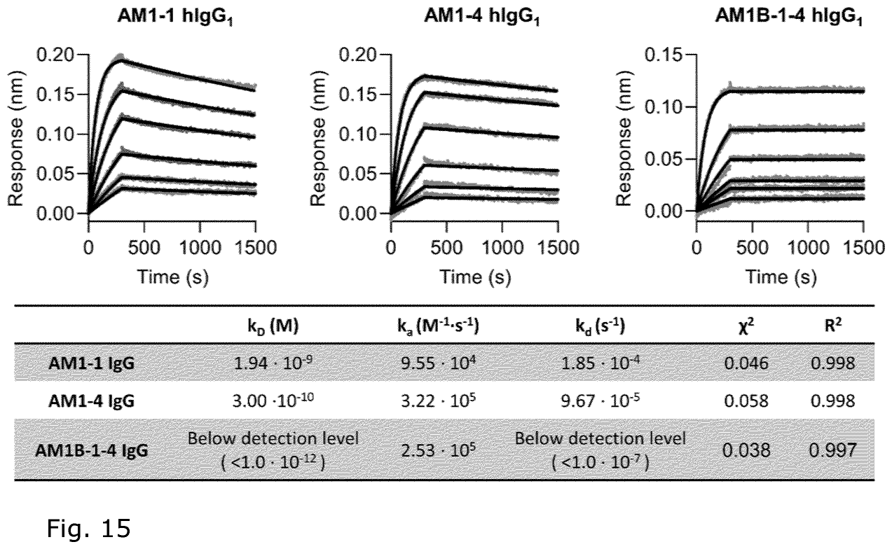

### FIG. 15

FIG. 15 shows binding Kinetics of Nanobody-based Human IgG1 formats. BLI analysis of two-fold dilution series of nApi m 1 binding to immobilized nanobody-based human IgG1. Kinetic constants were calculated using a 1:1 binding model (black lines).

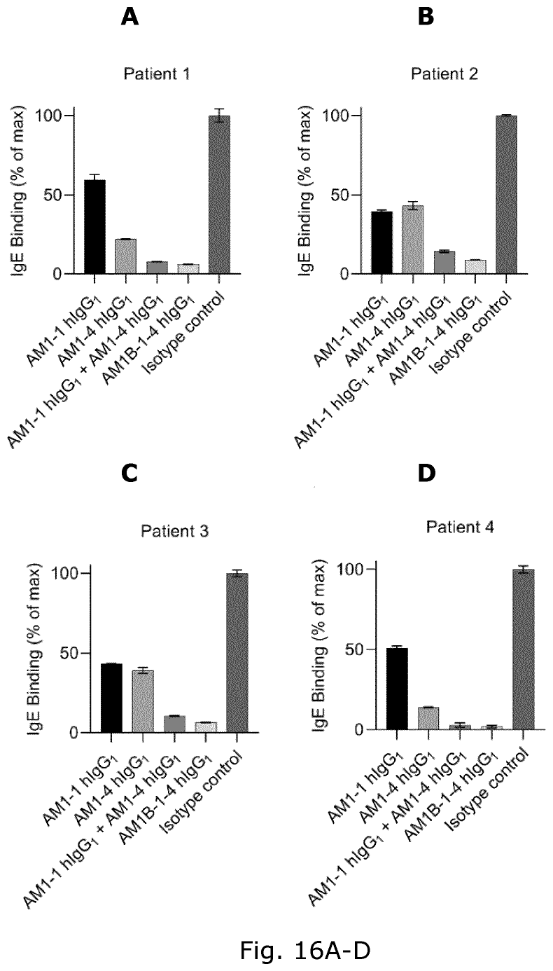

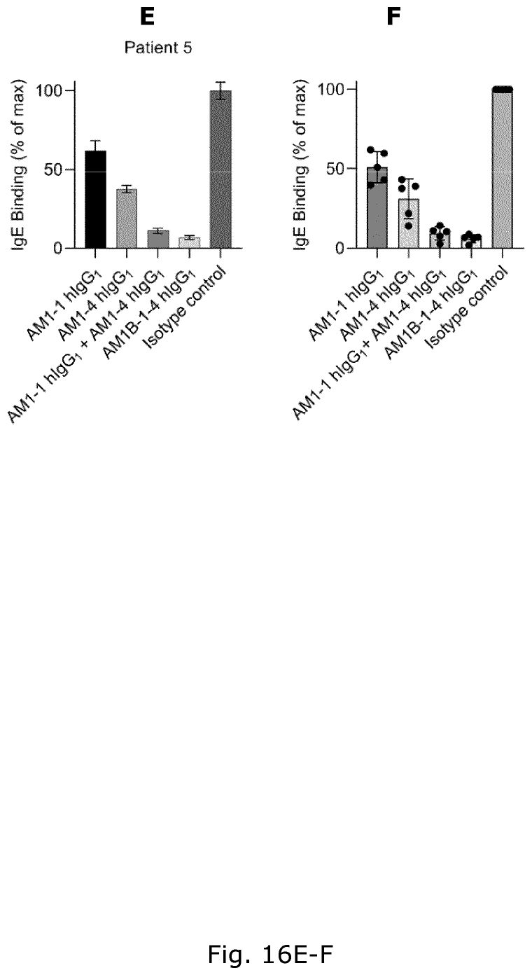

### FIG. 16

FIG. 16 shows inhibition of IgE binding to Api m 1. Binding of honeybee venom allergic patient IgE to immobilized Api m 1 was assessed in the presence of the different nanobody-based human IgG1 formats (1 μM). IgE binding was detected with anti-human IgE antibody conjugated to alkaline phosphatase. Sera from five patients were tested (A-E), the accumulative data is seen in F. Data are mean±s.d. of duplicates.

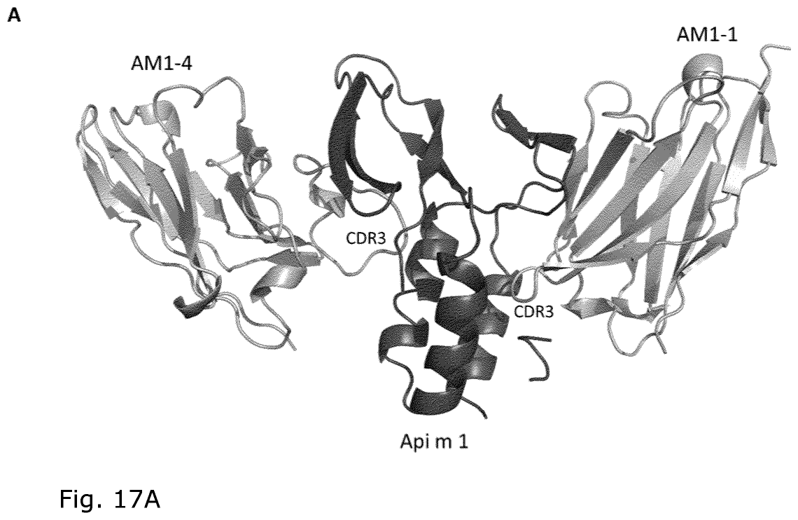

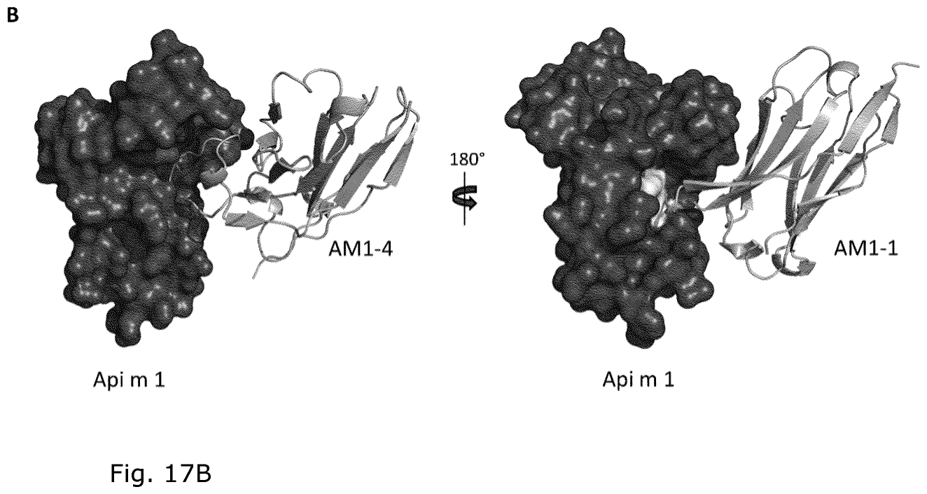

### FIG. 17

FIG. 17 shows a crystal structure of Api m 1 in complex with nanobodies AM1-1 and AM1-4. Cartoon representation (A) or surface representation (B) of Api m 1 in complex with nanobodies AM1-1 and AM1-4. Residues of the Api m 1 active site (H34, D64 and Y87) are shown in white.

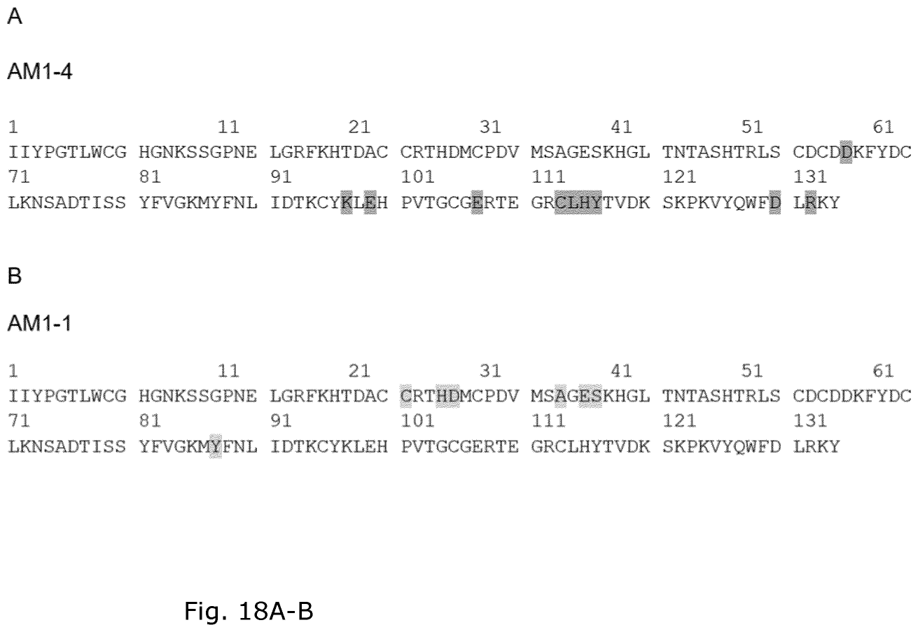

### FIG. 18

FIG. 18 shows the positions at which AM1-1 and AM1-4 binds to PLA2. A shows AM1-4 which corresponds to SEQ ID NO: 4, B shows AM1-1 which corresponds to SEQ ID NO: 1.

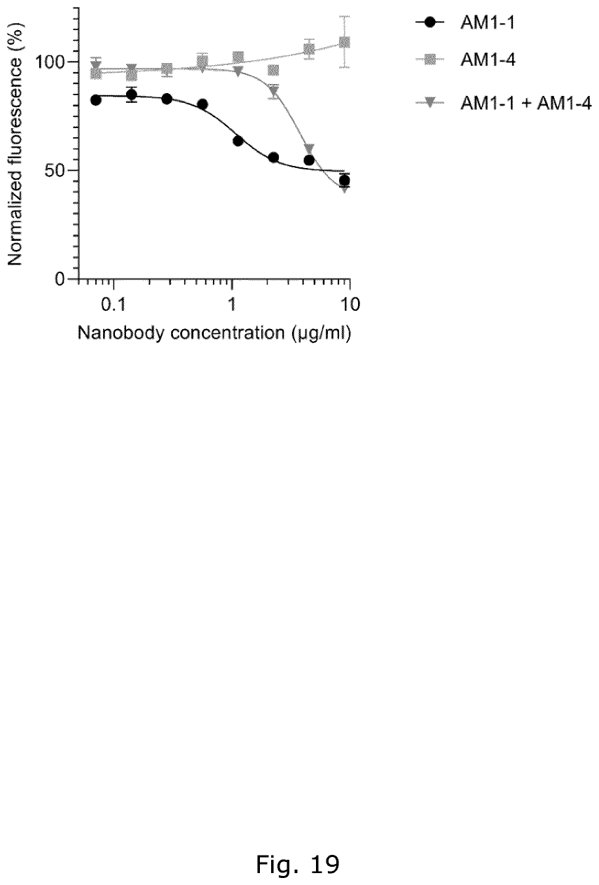

### FIG. 19

FIG. 19 shows the inhibition of the enzymatic activity of Api m 1 by AM1-1, AM1-4 or the combination thereof. The enzymatic activity is assessed using the phospholipase activity assay of native Api m 1 from honeybee venom in the presence of the nanobodies AM1-1 and AM1-4. Activity was measured as fluorescence using the Phospholipase A2 substrate Red/Green BODIPY® PC-A2 (Invitrogen). Data are mean±s.d. of duplicates.

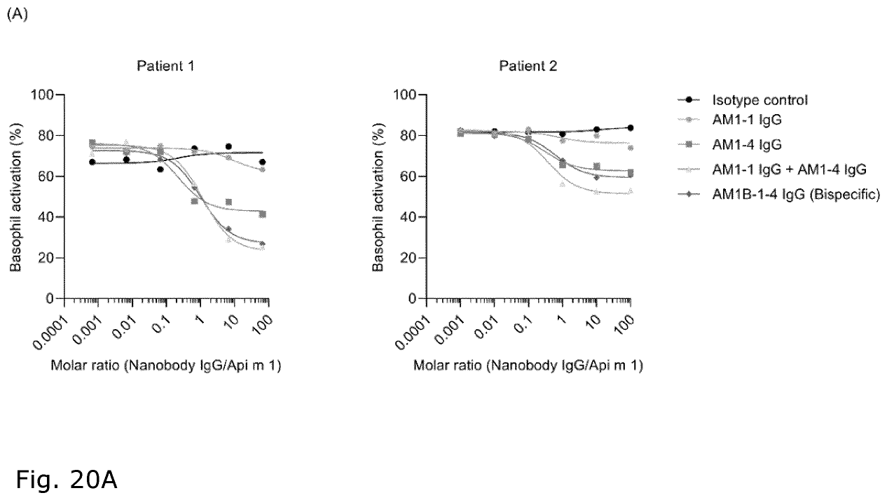

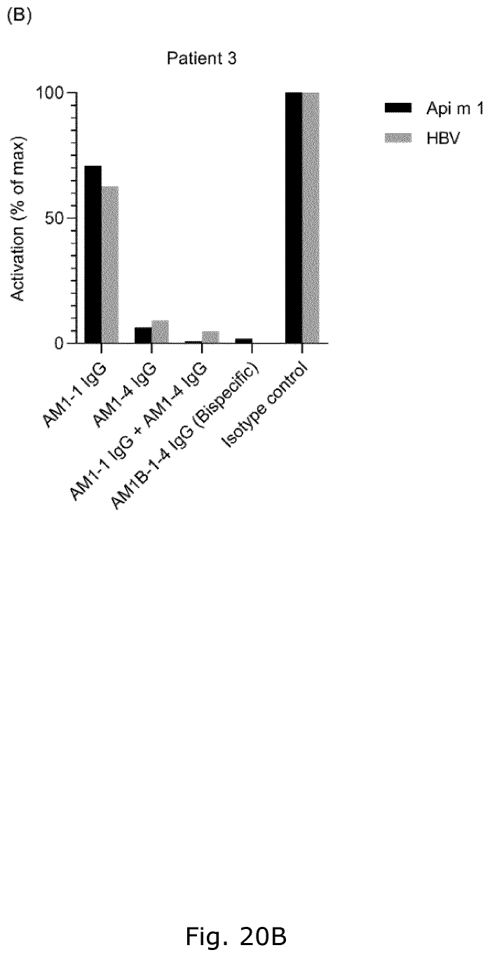

### FIG. 20

FIG. 20 shows the inhibition of basophil activation by nanobody-based hIgG₁. The basophil activation test was performed to assess the potential to reduce activation of basophils from honeybee venom allergic patients. (A) A fixed concentration of Api m 1 was incubated with a 10-fold dilution series of nb-IgG before incubation with EDTA blood from a honeybee venom allergic patient. (B) A 10-fold excess of nb-hIgG was incubated with a fixed concentration of Api m 1 or honeybee venom (HBV) before incubation with EDTA blood from a honeybee venom allergic patient. Activated basophils were identified as CD63 positive.

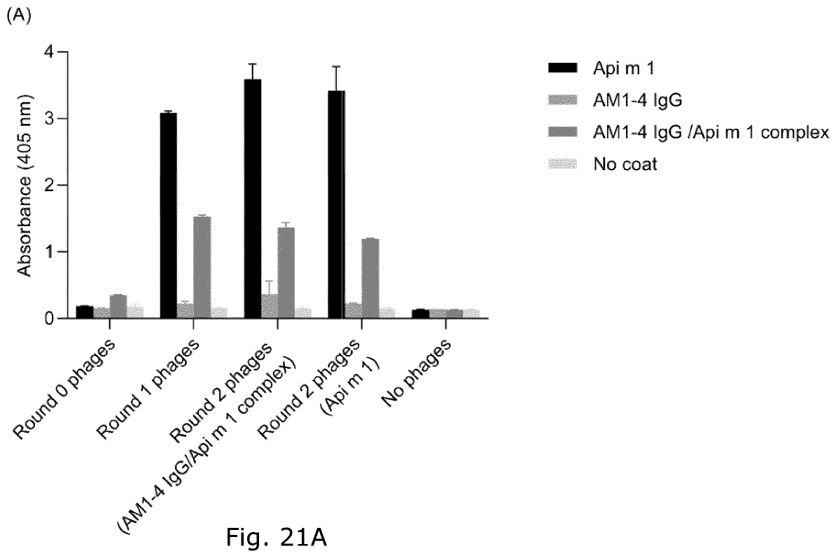

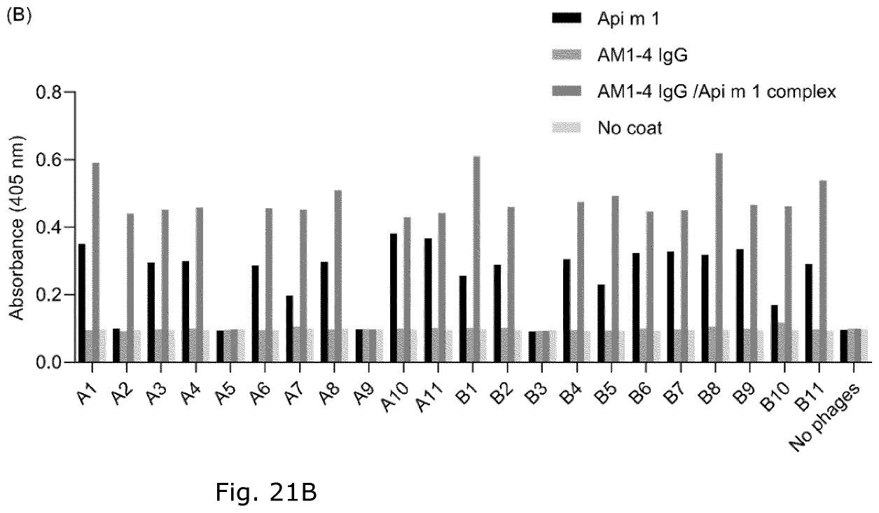

### FIG. 21

FIG. 21 shows a selection of nanobodies binding to Api m 1 in complex with AM1-4 IgG. Enrichment of the nanobody phage library was assessed in ELISA by measuring the immunoreactivity of polyclonal phages (A). Binding of phages to immobilized Api m 1 or AM1-4 IgG/Api m 1 complexes was detected using a biotinylated anti-M13 antibody. Data are mean±s.d. of duplicates. Immunoreactivity of monoclonal phages was analyzed in ELISA as above (B). Data are single measurements.

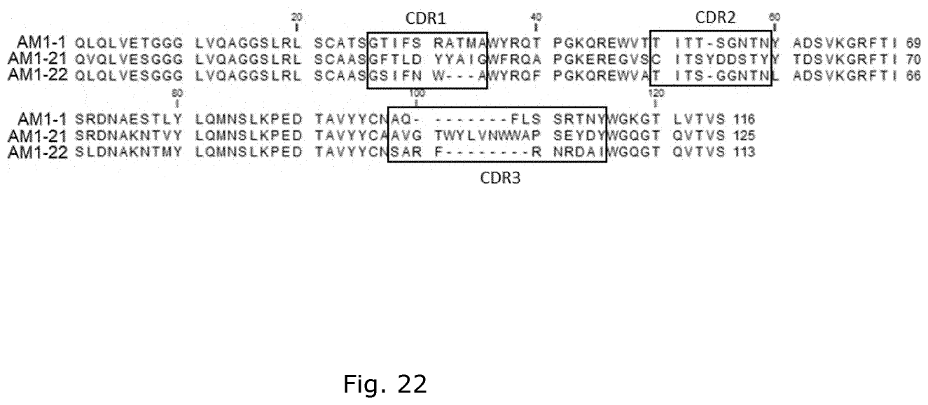

### FIG. 22

FIG. 22 shows sequence alignment of the three nanobodies selected against AM1-4 IgG/Api m 1 complexes. The Complementarity-determining regions (CDRs) are highlighted. AM1-1 (SEQ ID NO: 1), AM1-21 (SEQ ID NO: 71), and AM1-22 (SEQ ID NO: 72).

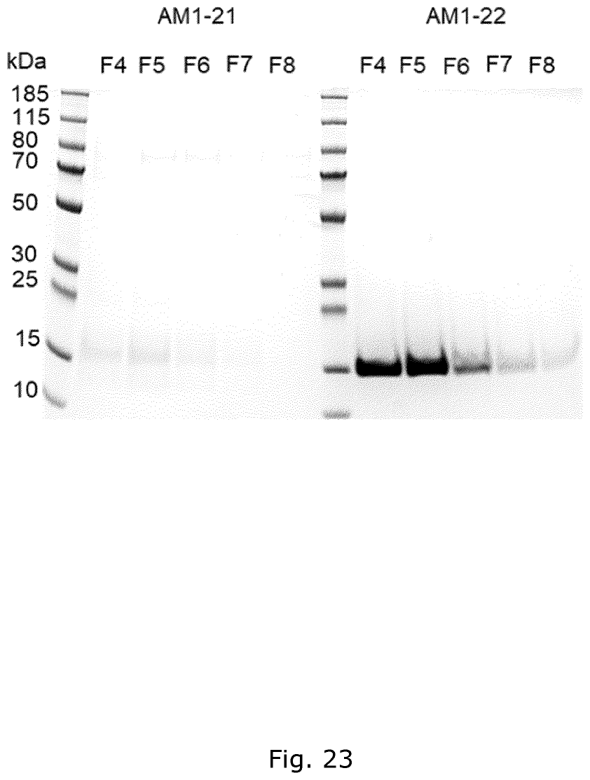

### FIG. 23

FIG. 23 shows an SDS-PAGE gel of purified nanobodies. Expressed nanobodies were purified from the supernatant by immobilized metal affinity chromatography. Fractions (F) from the purification were assessed by SDS-PAGE.

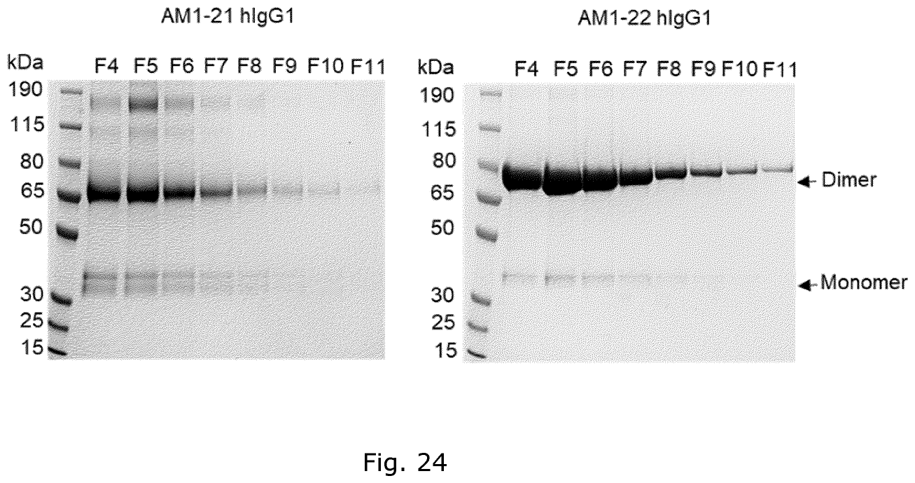

### FIG. 24

FIG. 24 shows two SDS-PAGE gels of purified nanobody-based human IgG1. Expressed nanobodies were purified from the supernatant of transient transfected cells by immobilized metal affinity chromatography. Fractions (F) from the purification were assessed by SDS-PAGE.

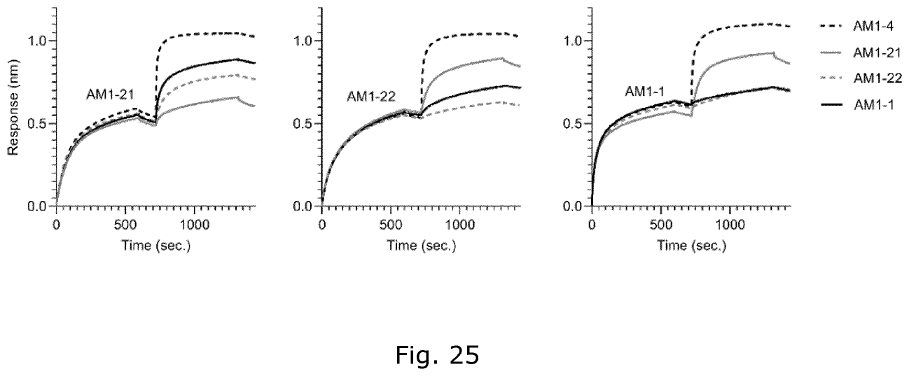

### FIG. 25

FIG. 25 shows the results from sequential biolayer interferometry analysis of nanobodies binding to immobilized Api m 1. Nanobody association was followed by a short dissociation and association of a second nanobody.

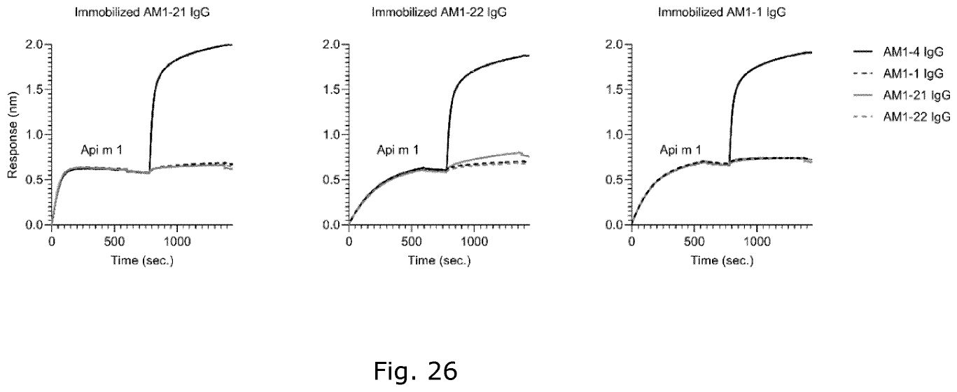

### FIG. 26

FIG. 25 shows the results from sequential biolayer interferometry analysis of nanobody-based human IgG1 binding to Api m 1. Nanobody-based human IgG1 was immobilized onto the sensors. Api m 1 association was followed by a short dissociation and association of a second nanobody-based human IgG1.

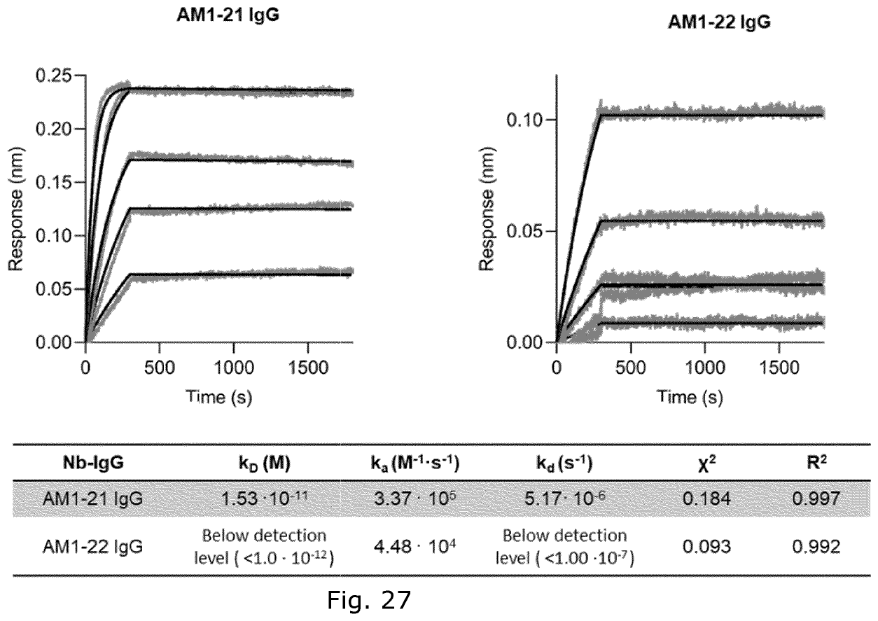

### FIG. 27

FIG. 27 shows the binding Kinetics of Nanobody-based Human IgG1 formats. BLI analysis of two-fold dilution series of nApi m 1 binding to immobilized nanobody-based human IgG1. Kinetic constants were calculated using a 1:1 binding model (black lines).

The present invention will now be described in more detail in the following.

### DETAILED DESCRIPTION OF THE INVENTION

### Definitions

Prior to discussing the present invention in further details, the following terms and conventions will first be defined:

### Single Domain Antibody

A single-domain antibody (sdAb), also known as a “nanobody”, is an antibody fragment consisting of a single monomeric variable antibody domain. Like a whole antibody, it is able to bind selectively to a specific antigen. With a molecular weight of only around 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain).

The single domain antibodies (such as a nanobody) of the invention bind selectively to (honey) bee “Api m 1” (PLA2) or (honey) bee “Api m 2” that is they bind preferentially to (honey) bee “Api m 1” (PLA2) (uniprot ID no P00630) or (honey) bee “Api m 2” with a greater binding affinity than to other antigens. The antibodies may bind selectively to (honey) bee “Api m 1” (PLA2) or (honey) bee “Api m 2”, but also bind detectably to other homologues hymenoptera venom allergen “Api m 1” or “Api m 2”.

In the present context, the terms “nanobody” and “single domain antibodies” are used interchangeably.

### Passive Immunity

In the present context, the term “passive immunity” is to be understood as administering to a subject (single domain) antibodies to prevent, treat or alleviate a disease (such as bee allergy) rather than the antibodies being produced by the subject's own immune system.

Passive immunisation by injecting allergen-specific antibodies is a promising approach for seasonal protection and treatment of allergies, such as bee venom allergy, more specifically honeybee venom allergy.

### Effective Amount

The terms “effective amount” and “therapeutically effective amount”, refer to an amount of a selective binding agent that is useful or necessary to support an observable change in the level of one or more biological activities of the single domain antibodies according to the invention.

### Sequence Identity

In the context of the present invention, the term “sequence identity” or “homologue” indicates a quantitative measure of the degree of homology between two amino acid sequences or between two nucleic acid sequences. If the two sequences to be compared are not of equal length, they must be aligned to give the best possible fit, allowing the insertion of gaps or, alternatively, truncation at the ends of the polypeptide sequences or nucleotide sequences. The sequence identity can be calculated as

$$\frac{( {N}_{ref} - {N}_{dif} ) 100}{{N}_{nef}} ,$$

wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (Ndif=2 and Nref=8). A gap is counted as non-identity of the specific residue(s), i.e. the DNA sequence AGTGTC will have a sequence identity of 75% with the DNA sequence AGTCAGTC (N_{dif}=2 and N_{ref}=8).

With respect to all embodiments of the invention relating to amino acid sequences or nucleotide sequences, the percentage of sequence identity between one or more sequences may also be based on alignments using the clustalW software (http:/www.ebi.ac.uk/clustalW/index.html) with default settings. For nucleotide sequence alignments these settings are: Alignment=3Dfull, Gap Open 10.00, Gap Ext. 0.20, Gap separation Dist. 4, DNA weight matrix: identity (IUB). For amino acid sequence alignments the settings are as follows: Alignment=3Dfull, Gap Open 10.00, Gap Ext. 0.20, Gap separation Dist. 4, Protein weight matrix: Gonnet.

Alternatively, nucleotide sequences may be analysed using programme DNASIS Max and the comparison of the sequences may be done at http://www.paralign.org/. This service is based on the two comparison algorithms called Smith-Waterman (SW) and ParAlign. The first algorithm was published by Smith and Waterman (1981) and is a well-established method that finds the optimal local alignment of two sequences. The other algorithm, ParAlign, is a heuristic method for sequence alignment; details on the method are published in Rognes (2001). Default settings for score matrix and Gap penalties as well as E-values were used.

### KD

In the present context, the terms “KD” or “KD value” refer to the equilibrium dissociation constant between an antibody (single-domain antibody) and its antigen. The KD value relates to the concentration of antibody (the amount of antibody needed for a particular experiment) and so the lower the KD value (lower concentration) and thus the higher the affinity of the antibody. In the present context, KD may be measured by Biacore T200. In an alternative embodiment, KD may be measured by BLI measurements on an Octet RED96 machine (ForteBio).

### Single Domain Antibody

As outlined above, the present invention relates to passive immunisation by administering allergen-specific antibodies/nanobodies for protection, alleviation and treatment of allergies, such as bee venom allergy, and in particular honeybee venom allergy. Thus, an aspect of the invention relates to a single domain antibody having binding affinity for Api m 1 (PLA2), wherein said single domain antibody comprises CDR domains selected from the group consisting of:
i. CDR domains:
   CDR1 according to SEQ ID NO: 13;
   CDR2 according to SEQ ID NO: 14; and
   CDR3 according to SEQ ID NO: 15;
ii. CDR domains:
   CDR1 according to SEQ ID NO: 16;
   CDR2 according to SEQ ID NO: 17; and
   CDR3 according to SEQ ID NO: 18;
iii. CDR domains:
   CDR1 according to SEQ ID NO: 19;
   CDR2 according to SEQ ID NO: 20; and
   CDR3 according to SEQ ID NO: 21;
iv. CDR domains:
   CDR1 according to SEQ ID NO: 22;
   CDR2 according to SEQ ID NO: 23; and
   CDR3 according to SEQ ID NO: 24;
v. CDR domains:
   CDR1 according to SEQ ID NO: 25;
   CDR2 according to SEQ ID NO: 26; and
   CDR3 according to SEQ ID NO: 27;
vi. CDR domains:
   CDR1 according to SEQ ID NO: 28;
   CDR2 according to SEQ ID NO: 29; and
   CDR3 according to SEQ ID NO: 30;
vii. CDR domains:
   CDR1 according to SEQ ID NO: 31;
   CDR2 according to SEQ ID NO: 32; and
   CDR3 according to SEQ ID NO: 33.
viii. CDR domains:
   CDR1 according to SEQ ID NO: 34;
   CDR2 according to SEQ ID NO: 35; and
   CDR3 according to SEQ ID NO: 36;
ix. CDR domains:
   CDR1 according to SEQ ID NO: 37;
   CDR2 according to SEQ ID NO: 38; and
   CDR3 according to SEQ ID NO: 39;
x. CDR domains:
   CDR1 according to SEQ ID NO: 40;
   CDR2 according to SEQ ID NO: 41; and
   CDR3 according to SEQ ID NO: 42;
xi. CDR domains:
   CDR1 according to SEQ ID NO: 43;
   CDR2 according to SEQ ID NO: 44; and
   CDR3 according to SEQ ID NO: 45;
xii. CDR domains:
   CDR1 according to SEQ ID NO: 46;
   CDR2 according to SEQ ID NO: 47; and
   CDR3 according to SEQ ID NO: 48;
xiii. CDR domains:
   CDR1 according to SEQ ID NO: 73;
   CDR2 according to SEQ ID NO: 74; and
   CDR3 according to SEQ ID NO: 75;
xiv. CDR domains:
   CDR1 according to SEQ ID NO: 76;
   CDR2 according to SEQ ID NO: 77; and
   CDR3 according to SEQ ID NO: 78;
or single domain antibodies according to any of i.-xii. having 1-3 substitutions in one or more of the CDR1, CDR2 and CDR3 domains, preferably 1-2 substitutions and more preferably 1 substitution in one or more of the CDR1, CDR2 and CDR3 domains;

### OR

a single domain antibody having binding affinity for Api m 2, wherein said single domain antibody comprises CDR domains selected from the group consisting of:
xv. CDR domains:
   CDR1 according to SEQ ID NO: 54;
   CDR2 according to SEQ ID NO: 55; and
   CDR3 according to SEQ ID NO: 56;
xvi. CDR domains:
   CDR1 according to SEQ ID NO: 57;
   CDR2 according to SEQ ID NO: 58; and
   CDR3 according to SEQ ID NO: 59;
xvii. CDR domains:
   CDR1 according to SEQ ID NO: 60;
   CDR2 according to SEQ ID NO: 61; and
   CDR3 according to SEQ ID NO: 62;
xviii. CDR domains:
   CDR1 according to SEQ ID NO: 63;
   CDR2 according to SEQ ID NO: 64; and
   CDR3 according to SEQ ID NO: 65;
xix. CDR domains:
   CDR1 according to SEQ ID NO: 66;
   CDR2 according to SEQ ID NO: 67; and
   CDR3 according to SEQ ID NO: 68;
or single domain antibodies according to any of xiii.-xvii. having 1-3 substitutions in one or more of the CDR1, CDR2 and CDR3 domains, preferably 1-2 substitutions and more preferably 1 substitution in one or more of the CDR1, CDR2 and CDR3 domains.

As outlined in the example section, single domain antibodies with the above outlined CDR domains have been identified and characterized.

In a preferred embodiment, said single domain antibody comprises CDR domains selected from the group consisting of:
i. CDR domains:
   CDR1 according to SEQ ID NO: 13;
   CDR2 according to SEQ ID NO: 14; and
   CDR3 according to SEQ ID NO: 15;
ii. CDR domains:
   CDR1 according to SEQ ID NO: 16;
   CDR2 according to SEQ ID NO: 17; and
   CDR3 according to SEQ ID NO: 18;
iii. CDR domains:
   CDR1 according to SEQ ID NO: 19;
   CDR2 according to SEQ ID NO: 20; and
   CDR3 according to SEQ ID NO: 21;
iv. CDR domains:
   CDR1 according to SEQ ID NO: 22;
   CDR2 according to SEQ ID NO: 23; and
   CDR3 according to SEQ ID NO: 24;
v. CDR domains:
   CDR1 according to SEQ ID NO: 25;
   CDR2 according to SEQ ID NO: 26; and
   CDR3 according to SEQ ID NO: 27;
vi. CDR domains:
   CDR1 according to SEQ ID NO: 28;
   CDR2 according to SEQ ID NO: 29; and
   CDR3 according to SEQ ID NO: 30;
vii. CDR domains:
   CDR1 according to SEQ ID NO: 31;
   CDR2 according to SEQ ID NO: 32; and
   CDR3 according to SEQ ID NO: 33.
viii. CDR domains:
   CDR1 according to SEQ ID NO: 34;
   CDR2 according to SEQ ID NO: 35; and
   CDR3 according to SEQ ID NO: 36;
ix. CDR domains:
   CDR1 according to SEQ ID NO: 37;
   CDR2 according to SEQ ID NO: 38; and
   CDR3 according to SEQ ID NO: 39;
x. CDR domains:
   CDR1 according to SEQ ID NO: 40;
   CDR2 according to SEQ ID NO: 41; and
   CDR3 according to SEQ ID NO: 42;
xi. CDR domains:
   CDR1 according to SEQ ID NO: 43;
   CDR2 according to SEQ ID NO: 44; and
   CDR3 according to SEQ ID NO: 45;
xii. CDR domains:
   CDR1 according to SEQ ID NO: 46;
   CDR2 according to SEQ ID NO: 47; and
   CDR3 according to SEQ ID NO: 48;
xiii. CDR domains:
   CDR1 according to SEQ ID NO: 73;
   CDR2 according to SEQ ID NO: 74; and
   CDR3 according to SEQ ID NO: 75;
xiv. CDR domains:
   CDR1 according to SEQ ID NO: 76;
   CDR2 according to SEQ ID NO: 77; and
   CDR3 according to SEQ ID NO: 78;

### OR

a single domain antibody having binding affinity for Api m 2, wherein said single domain antibody comprises CDR domains selected from the group consisting of:
xv. CDR domains:
   CDR1 according to SEQ ID NO: 54;
   CDR2 according to SEQ ID NO: 55; and
   CDR3 according to SEQ ID NO: 56;
xvi. CDR domains:
   CDR1 according to SEQ ID NO: 57;
   CDR2 according to SEQ ID NO: 58; and
   CDR3 according to SEQ ID NO: 59;
xvii. CDR domains:
   CDR1 according to SEQ ID NO: 60;
   CDR2 according to SEQ ID NO: 61; and
   CDR3 according to SEQ ID NO: 62;
xviii. CDR domains:
   CDR1 according to SEQ ID NO: 63;
   CDR2 according to SEQ ID NO: 64; and
   CDR3 according to SEQ ID NO: 65;
xix. CDR domains:
   CDR1 according to SEQ ID NO: 66;
   CDR2 according to SEQ ID NO: 67; and
   CDR3 according to SEQ ID NO: 68.

The single domain antibodies having specificity for Api m 1, may also be characterized by the full length sequence. Thus, in an embodiment, the single domain antibody is selected from the group consisting of
a) SEQ ID NO's: 1-12 and 71-72; or
b) a single domain antibody having at least 70%, such at least 80%, for example at least 85%, preferably at least 90% or more than 95% sequence identity, such as 100% sequence identity with the amino acid according to a), with the proviso that said single domain antibody comprises a CDR1 domain, a CDR2 domain and a CDR3 domain according to any of i.-xiv. as defined above.

In an embodiment, the single domain antibody has at least 90% sequence identity or more than 95% sequence identity, such as 100% sequence identity with the amino acid according to a), with the proviso that said single domain antibody comprises a CDR1 domain, a CDR2 domain and a CDR3 domain according to any of i.-xiv. as defined above.

SEQ ID NO's: 1-12 and 71-72 correspond to the full-length single domain antibodies having affinity for Api m 1.

In a preferred embodiment, when a substitution is made to the single domain antibody of group I, Tyr37, Thr50, Asn58, Ser101 and Arg103 of SEQ ID NO: 1 are maintained, i.e. the Ser at position 5 and the Arg at position 7 in SEQ ID NO: 15, the Asp at position 9 of SEQ ID NO: 14 and the Tyr and Thr at positions 37 and 50 in SEQ ID NO: 1. More specifically, a single domain antibody having binding affinity for Api m 1 (PLA2), wherein said single domain antibody comprises a Tyr and Thr at positions 37 and 50 according to SEQ ID NO: 1, and wherein said single domain antibody comprises CDR domains:
CDR1 according to SEQ ID NO: 13, the CDR1 domain having 1-3 substitutions, preferably 1-2 substitutions and more preferably 1 substitution;
CDR2 according to SEQ ID NO: 14, the CDR2 domain having 1-3 substitutions, preferably 1-2 substitutions and more preferably 1 substitution, with the proviso that the Asp at position 9 of SEQ ID NO: 14 is maintained; and
CDR3 according to SEQ ID NO: 15, the CDR3 domain having 1-3 substitutions, preferably 1-2 substitutions and more preferably 1 substitution, with the proviso that the Ser at position 5 and the Arg at position 7 in SEQ ID NO: 15 are maintained;
In another preferred embodiment, when a substitution is made to the single domain antibody of group IV, Gly103 to Arg 107 of SEQ ID NO: 4 are maintained, i.e. the linear peptide GlySerSerThrArg in CDR3 (SEQ ID NO: 24).

More specifically, a single domain antibody having binding affinity for Api m 1 (PLA2), wherein said single domain antibody comprises CDR domains:
CDR1 according to SEQ ID NO: 22, the CDR1 domain having 1-3 substitutions, preferably 1-2 substitutions and more preferably 1 substitution;
CDR2 according to SEQ ID NO: 23, the CDR2 domain having 1-3 substitutions, preferably 1-2 substitutions and more preferably 1 substitution; and
CDR3 according to SEQ ID NO: 24, the CDR3 domain having 1-3 substitutions, preferably 1-2 substitutions and more preferably 1 substitution, with the proviso that the sequence GlySerSerThrArg of SEQ ID NO: 15 is maintained;

The single domain antibodies having specificity for Api m 2, may also be characterized by the full length sequence. Thus, in an embodiment, the single domain antibody is elected from the group consisting of
a) SEQ ID NO's 49-53; or
b) a single domain antibody having at least 70%, such at least 80%, for example at least 85%, such as at least 90% or more than 95% sequence identity (such as 100%) with the amino acid according to a), with the proviso that said single domain antibody comprises a CDR1 domain, a CDR2 domain and a CDR3 domain according to any of xv.-xix. as defined in claim 1 or 2.

In an embodiment, the single domain antibody has at least 90% sequence identity or more than 95% sequence identity, such as 100% sequence identity with the amino acid according to a), with the proviso that said single domain antibody comprises a CDR1 domain, a CDR2 domain and a CDR3 domain according to any of xiii.-xvii. as defined above.

SEQ ID NO's: 49-53 correspond to the full-length single domain antibodies having affinity for Api m 2.

The single domain antibody according to the invention should have a certain binding affinity for their targets. Thus, in an embodiment, the single domain antibody has a K_{D} value to Api m 1 below 1*10⁻⁷, preferably below 1*10⁻⁸, or more preferably below 1*10⁻⁹ M, or such as in the range 1*10⁻⁷ to 1*10⁻¹² M, or preferably in the range 1*10⁻⁸ to 1*10⁻¹⁰ M.

In a corresponding embodiment, the single domain antibody has a K_{D} value to Api m 2 below 1*10⁻⁷, preferably below 1*10⁻⁸, or more preferably below 1*10⁻⁹ M, or such as in the range 1*10⁻⁷ to 1*10⁻¹² M, or preferably in the range 1*10⁻⁸ to 1*10⁻¹⁰ M.

In example 17, it is shown that AM1-21 IgG and AM1-22 IgG bind nApi m 1 with high affinities of 1.53·10⁻¹¹ M and below detection level (1.0·10⁻¹² M) respectively.

In another embodiment, the single domain antibody is humanized.

In yet another embodiment, the single domain antibody has a maximum length of 150 amino acids, preferably at the most 140 amino acids, more preferably at the most 130 amino acids.

In a further embodiment, the single domain antibody is a Nanobody or a VHH antibody.

The single domain antibody annotated as AM1-1 shows unique properties, including binding to an epitope on Api m 1 (from the honeybee) different from the other identified antibodies (see example 3). The epitope is located at the active site, and thus in a preferred embodiment, the single domain antibody of the invention binds to the active site, i.e. one or more of the residues Cys31, His34, Asp35, Ala43, Glu45, Ser46, and Tyr87, preferably His34, and Tyr87 of SEQ ID NO: 70. Thus, in a preferred embodiment the single domain antibody comprises the CDR domains
CDR1 according to SEQ ID NO: 13;
CDR2 according to SEQ ID NO: 14; and
CDR3 according to SEQ ID NO: 15.

In another preferred embodiment, the single domain antibody comprises SEQ ID NO: 1.

The single domain antibodies AM1-21 and AM1-22 has been characterized as outlined in examples 14-17, showing that these antibodies have unique properties compared to the other known antibodies having affinity for Api m 1.

Thus, in an embodiment, the single domain antibody comprises the CDR domains (AM1-21):
CDR1 according to SEQ ID NO: 73;
CDR2 according to SEQ ID NO: 74; and
CDR3 according to SEQ ID NO: 75.

In another preferred embodiment, the single domain antibody comprises SEQ ID NO: 71. (AM1-21 full length).

In another embodiment, the single domain antibody comprises the CDR domains (AM1-22):
CDR1 according to SEQ ID NO: 76;
CDR2 according to SEQ ID NO: 77; and
CDR3 according to SEQ ID NO: 78.

In another preferred embodiment, the single domain antibody comprises SEQ ID NO: 72. (AM1-22 full length).

The single domain antibody annotated as AM1-4 binds to an epitope positioned on the opposite side of Api m 1 (from the honeybee). Thus, in another embodiment, the single domain antibody of the invention binds to Asp65, Lys97, Glu99, Glu107, Cys113, Leu114, His115, Tyr116, Asp130, and Arg132 of SEQ ID NO: 70.

The single domain antibodies according to the invention may be coupled to different moieties. Thus, in an embodiment, the single domain antibody is coupled to an Ig, such as IgE or IgG, such as IgG4 or IgG1, preferably to the Fc part of the Ig, such as the Fc part of IgG or the Fc part of IgE.

In another embodiment, the single domain antibody is coupled to another proteinaceous or non-proteinaceous entity.

In yet an embodiment, the proteinaceous entity is selected from the group consisting of a single domain antibody, or other proteinaceous antigen binders including but not limited to antibody fragments, darpins, lipocalins, etc., half-life extending entities such as BSA, and functional peptides for targeted delivery.

In yet another embodiment, the non-proteinaceous entity is selected from the group consisting of a half-life extending entity, such as polyethyleneglycol (PEG), or similar, or to moieties for targeted delivery such as carbohydrates.

### Epitope

The inventors of the present invention have developed a single domain antibody, targeting the active site of Api m 1 (PLA2). Thus, in one embodiment, the single domain antibody of the invention relates to a single domain antibody targeting the active site of Api m 1 (PLA2). The active site in Api m 1 (PLA2) comprises the catalytic residues H34, D64 and Y87, thus by targeting one or more of these residues the catalytic activity of Api m 1 (PLA2) will be reduced, due to their close proximity (FIG. 17B). Reducing the catalytic activity will also reduce the toxic effect Api m 1 (PLA2) can exert in a subject. Thus, in a preferred embodiment, the single domain antibody of the invention binds to the active site, i.e. one or more, such as 1, 2, 3, 4, 5, 6, or all of the residues Cys31, His34, Asp35, Ala43, Glu45, Ser46, and Tyr87, preferably His34, and Tyr87 according to SEQ ID NO: 70.

In a specific embodiment, the antibody binds to the residues Cys31, His34, Asp35, Ala43, Glu45, Ser46, and Tyr87 of SEQ ID NO: 70. In another embodiment, the single domain antibody binding to the active site is the single domain antibody of group I, comprising CDRs 1-3 according to SEQ ID NO: 13-15 or the single domain antibody comprising SEQ ID NO: 1, as defined in any of the embodiments further herein.

The inventors of the present invention have additionally developed single domain antibodies binding to a site different from the active site. As seen from FIG. 17 such a site can be located on the opposite side of Api m 1. In an embodiment, the invention relates to antibodies binding to one or more, such as 1, 2, 3, 4, 5, 6, 7, 8, 9, or all of the residues Asp65, Lys97, Glu99, Glu107, Cys113, Leu114, His115, Tyr116, Asp130, and Arg132 of SEQ ID NO: 70. In one embodiment, a single domain antibody binding to such a site is an antibody selected from the group consisting of AM1-3, AM1-4, and AM1-6, i.e. an antibody comprising:
iii. CDR domains:
   CDR1 according to SEQ ID NO: 19;
   CDR2 according to SEQ ID NO: 20; and
   CDR3 according to SEQ ID NO: 21;
iv. CDR domains:
   CDR1 according to SEQ ID NO: 22;
   CDR2 according to SEQ ID NO: 23; and
   CDR3 according to SEQ ID NO: 24; or
vi. CDR domains:
   CDR1 according to SEQ ID NO: 28;
   CDR2 according to SEQ ID NO: 29; and
   CDR3 according to SEQ ID NO: 30;
or a single domain antibody selected from SEQ ID NO: 3, 4, or 6, as defined in any of the embodiments further herein.

In a preferred embodiment, the single domain antibody binding to one or more, such as 1, 2, 3, 4, 5, 6, 7, 8, 9, or all of the residues Asp65, Lys97, Glu99, Glu107, Cys113, Leu114, His115, Tyr116, Asp130, and Arg132 of SEQ ID NO: 70 is AM1-4, i.e. group iv and/or SEQ ID NO: 4 as defined above.

The skilled person will recognize that by selecting single domain antibodies capable of binding to distinct epitopes, a multi-specific antibody can be generated with increased avidity towards an allergen. Multi-specific antibodies are defined further herein.

### Compositions and Combinations

The single domain antibodies according to the invention may preferably be included in compositions or combinations. Thus, an aspect of the invention relates to a composition or combination comprising one or more of the single domain antibodies according to the invention.

In an embodiment, the composition or combination comprises one or more of the single domain antibodies having binding affinity for Api m 1 according to the invention.

In yet an embodiment, the composition or combination comprises two or more of the single domain antibodies having binding affinity for Api m 1 according to the invention, such as 3-12, such as 4-12, such as 5-12, such as 3-10, or such as 3-5, or preferably 2-3 single domain antibodies.

In yet another embodiment, the composition or combination according to the invention, comprises at least a single domain antibody comprising
CDR domains:
   CDR1 sequence according to SEQ ID NO: 13;
   CDR2 sequence according to SEQ ID NO: 14; and
   CDR3 sequence according to SEQ ID NO: 15.

Again, such as single domain antibody has been annotated AM1-1 and binds to a unique epitope on Api m 1 (see example 3).

In a further embodiment, the composition or combination according to the invention comprises at least a single domain antibody comprising SEQ ID NO: 1. SEQ ID NO: 1 corresponds to full length AM1-1.

In yet an embodiment, the composition or combination according to the invention, comprises a further antibody, such as a single domain antibody, having affinity for Api m 1, wherein said further single domain antibody binds to an epitope different from the epitope targeted by AM1-1, such as a non-overlapping epitope. By including several antibodies targeting different epitopes on the same target molecule, a higher inhibitory effect can be achieved (see example 7).

In another embodiment, the composition or combination according to the invention, comprises at least a single domain antibody comprising
CDR domains (AM1-21):
   CDR1 sequence according to SEQ ID NO: 73;
   CDR2 sequence according to SEQ ID NO: 74; and
   CDR3 sequence according to SEQ ID NO: 75.

In yet another embodiment, the composition or combination according to the invention, comprises at least a single domain antibody comprising
CDR domains (AM1-22):
   CDR1 sequence according to SEQ ID NO: 76;
   CDR2 sequence according to SEQ ID NO: 77; and
   CDR3 sequence according to SEQ ID NO: 78.

Again, such as single domain antibody has been annotated AM1-21 and AM1-22 respectively and bind to unique epitopes on Api m 1 (see example 16).

In a further embodiment, the composition or combination according to the invention comprises at least a single domain antibody comprising SEQ ID NO: 71. SEQ ID NO: 71 corresponds to full length AM1-21.

In yet a further embodiment, the composition or combination according to the invention comprises at least a single domain antibody comprising SEQ ID NO: 72. SEQ ID NO: 72 corresponds to full length AM1-22.

In yet an embodiment, the composition or combination according to the invention, comprises a further antibody, such as a single domain antibody, having affinity for Api m 1, wherein said further single domain antibody binds to an epitope different from the epitope targeted by AM1-21 and/or AM1-22, such as a non-overlapping epitope. By including several antibodies targeting different epitopes on the same target molecule, a higher inhibitory effect can be achieved (see e.g. example 7 and 16).

In another embodiment, the composition or combination according to the invention comprises one or more of the single domain antibodies having binding affinity for Api m 2.

In yet an embodiment, the composition or combination according to the invention comprises two or more of the single domain antibodies having binding affinity for Api m 2 according to the invention, such as 3-5, such as 4-5, preferably 2-3 single domain antibodies.

In yet another embodiment, the composition or combination according to the invention, comprises at least a single domain antibody comprising
CDR domains (AM2-A1):
   CDR1 sequence according to SEQ ID NO: 54;
   CDR2 sequence according to SEQ ID NO: 55; and
   CDR3 sequence according to SEQ ID NO: 56.
      and/or

### A Single Domain Antibody Comprising

CDR domains (AM2-C2):
   CDR1 sequence according to SEQ ID NO: 60;
   CDR2 sequence according to SEQ ID NO: 61; and
   CDR3 sequence according to SEQ ID NO: 62.

As outlined in example 5, the nanobodies AM2-A1 and AM2-C2 bind non-overlapping epitopes.

In a related embodiment, the composition or combination comprises at least a single domain antibody comprising SEQ ID NO: 49 (AM2-A1, full length) and/or a single domain antibody comprising SEQ ID NO: 51 (AM2-C2, full length).

In yet another embodiment, the composition or combination comprises a further antibody, such as a single domain antibody, having affinity for Api m 2, wherein said further single domain antibody binds to an epitope different from the single domain antibodies AM2-A1 and/or AM2-C2, such as an non-overlapping epitope.

In an embodiment, the composition or combination comprises one or more physiologically acceptable carriers, excipients and/or diluents.

### Multispecific/Bispecific Antibodies

To more efficiently target two epitopes on the same target, it may be advantageous to use multi-specific antibodies, such as a bispecific antibodies. Thus, an aspect of the invention relates to a multi-specific antibody, such as a bispecific antibody, comprising at least one of the single domain antibodies according to the invention. Example 6 and 8 shows generation of bispecific antibodies comprising single domain antibodies according to the invention, namely, bispecific nanobodies in a IgG₁-format comprising two different Api m 1 specific nanobodies. Example 7 and 10 shows nanobody-based inhibition demonstrating blocking of the binding of patient IgE to Api m 1.

Thus, in an embodiment, the multi-specific antibody comprises at least two of the single domain antibodies according to the invention.

In another embodiment, at least two of the entities of the multi-specific antibody target the same target (such as AP m 1 or AP m 2), preferably at different epitopes of the same target.

In yet another embodiment, at least two of the entities of the multi-specific antibody target different epitopes, such as targeting AP m 1 and AP m 2.

In a further embodiment, the multi-specific antibody, such as a bispecific antibody, comprises at least two of the single domain antibodies according to the invention.

In a preferred embodiment, the multi-specific antibody comprises a single domain antibody having the CDR domains (AM1-1):
CDR1 sequence according to SEQ ID NO: 13;
CDR2 sequence according to SEQ ID NO: 14; and
CDR3 sequence according to SEQ ID NO: 15;

In another preferred embodiment, the multi-specific antibody comprises a single domain antibody having the CDR domains (AM1-21):
CDR1 according to SEQ ID NO: 73;
CDR2 according to SEQ ID NO: 74; and
CDR3 according to SEQ ID NO: 75.

In a further preferred embodiment, the multi-specific antibody comprises SEQ ID NO: 71. (AM1-21 full length).

In yet another preferred embodiment, the multi-specific antibody comprises a single domain antibody having the CDR domains (AM1-22):
CDR1 according to SEQ ID NO: 76;
CDR2 according to SEQ ID NO: 77; and
CDR3 according to SEQ ID NO: 78.

In a further preferred embodiment, the multi-specific antibody comprises SEQ ID NO: 72. (AM1-22 full length).

In another preferred embodiment, the multi-specific antibody comprises a single domain antibody comprising SEQ ID NO: 1 (AM1-1), e.g. in combination with AM1-3 or a (single domain) antibody comprising the CDR regions of AM1-3. Such antibodies are generated and tested in examples 6-7.

In a further preferred embodiment, the multi-specific antibody comprises a single domain antibody comprising SEQ ID NO: 1 (AM1-1), e.g. in combination with AM1-4 or a single domain antibody comprising the CDR regions of AM1-4. Such antibodies are generated and tested in examples 8-10.

In another embodiment, the multi-specific antibody comprises a single domain antibody comprising SEQ ID NO: 1 (AM1-1), e.g. in combination with AM1-6 or a single domain antibody comprising the CDR regions of AM1-6. In an embodiment, the multi-specific antibody is IgG-based, such as IgG₁- or IgG₄-based. In examples 6-7 and 8-10, IgG₁-based bispecific antibodies are used.

In yet another embodiment, the multi-specific antibody comprises a single domain antibody comprising SEQ ID NO: 1 (AM1-1), e.g. in combination with AM1-21 and/or AM1-22 or a single domain antibody comprising the CDR regions of AM1-21 and/or AM1-22.

In another preferred embodiment, the multi-specific antibody comprises a single domain antibody comprising SEQ ID NO: 71 (AM1-21), e.g. in combination with AM1-3 or a (single domain) antibody comprising the CDR regions of AM1-3.

In a further preferred embodiment, the multi-specific antibody comprises a single domain antibody comprising SEQ ID NO: 72 (AM1-21), e.g. in combination with AM1-4 or a single domain antibody comprising the CDR regions of AM1-4.

In another embodiment, the multi-specific antibody comprises a single domain antibody comprising SEQ ID NO: 71 (AM1-21), e.g. in combination with AM1-6 or a single domain antibody comprising the CDR regions of AM1-6.

In yet another embodiment, the multi-specific antibody comprises a single domain antibody comprising SEQ ID NO: 71 (AM1-21), e.g. in combination with AM1-1 and/or AM1-22 or a single domain antibody comprising the CDR regions of AM1-1 and/or AM1-22.

In an embodiment, the multi-specific antibody is IgG-based, such as IgG₁- or IgG₄-based.

In another preferred embodiment, the multi-specific antibody comprises a single domain antibody comprising SEQ ID NO: 72 (AM1-22), e.g. in combination with AM1-3 or a (single domain) antibody comprising the CDR regions of AM1-3.

In a further preferred embodiment, the multi-specific antibody comprises a single domain antibody comprising SEQ ID NO: 72 (AM1-22), e.g. in combination with AM1-4 or a single domain antibody comprising the CDR regions of AM1-4. Such antibodies are generated and tested in examples 8-10.

In another embodiment, the multi-specific antibody comprises a single domain antibody comprising SEQ ID NO: 72 (AM1-22), e.g. in combination with AM1-6 or a single domain antibody comprising the CDR regions of AM1-6. In an embodiment, the multi-specific antibody is IgG-based, such as IgG₁- or IgG₄-based.

In yet another embodiment, the multi-specific antibody comprises a single domain antibody comprising SEQ ID NO: 72 (AM1-22), e.g. in combination with AM1-1 and/or AM1-21 or a single domain antibody comprising the CDR regions of AM1-1 and/or AM1-21.

Thus, in an embodiment, the multi-specific antibody is coupled to an Ig, such as IgE or IgG, such as IgG4 or IgG1, preferably to the Fc part of the Ig, such as the Fc part of IgG or the Fc part of IgE.

As visualized in example 11 and FIG. 17-18, the inventors have also found the specific binding sites of the single domain antibodies defined herein. An IgG is around 150 kDa compared to 15 kd Api m 1. By using IgG based multi-specific antibodies targeting two sites of Api m 1, and due to the flexibility of the arms and the hinges of the IgG the multi-specific antibody will be able to trap the allergen and protect it from contacting other binding partners.

### Medical Uses

The single domain antibodies according to the invention may find use as medicaments. Thus, an aspect of the invention relates to the single domain antibodies according to the invention, the multispecific antibodies according to the invention and/or the composition or combination according to the invention for use as a medicament. As outlined in the example section the antibodies according to the invention may have unique properties. For example, example 13 shows that the antibodies may inhibit basophil activation in honeybee venom allergic patients. Example 12 shows that the nanobody AM1-1 can reduce the enzymatic activity of Api m 1 to 50%.

In another aspect, the single domain antibodies according to the invention, the multispecific antibodies according to the invention and/or the composition or combination according to the invention is for use in the treatment, prevention and/or alleviation of bee venom allergy.

In an embodiment, the bee venom allergy is caused by the honeybee (Apis) venom.

In an embodiment the treatment, prevention and/or alleviation of bee venom allergy is caused by inhibition of basophil activation.

Being able to inhibit the enzymatic activity towards venom is also sought after. If a small molecule drug is used to inhibit a phospholipase venom, such as Api m 1, it is likely that the small molecule will also inhibit vital functions in the body performed by endogenous phospholipases-thus a highly specific single domain antibody or multispecific antibody, provides a much more targeted inhibition. Thus, in another aspect, the single domain antibodies according to the invention, the multispecific antibodies according to the invention and/or the composition or combination according to the invention is for use in the treatment, prevention and/or alleviation of a venom induced toxicity.

In another embodiment the treatment, prevention and/or alleviation of a venom induced toxicity is caused by reducing the enzymatic activity of Api m 1. Again reduced enzymatic activity may be due to blocking of the active site in Api m 1. In one embodiment, the venom inducing the toxicity is a honeybee (Apis) venom. In another embodiment, the venom inducing the toxicity is a phospholipase. In another embodiment, the venom inducing the toxicity is Api 1 m.

In another embodiment, the single domain antibodies according to the invention, the multispecific antibodies according to the invention and/or the composition or combination according to the invention are administered in a therapeutically effective amount.

In yet an embodiment, the single domain antibodies according to the invention, the multispecific antibodies according to the invention and/or the composition or combination according to the invention is for providing passive immunity against bee venom allergy.

In yet another embodiment, the single domain antibodies according to the invention, the multispecific antibodies according to the invention and/or the composition or combination according to the invention is administered before the bee season, such as in late spring, such as April-June in relation to the Nordic hemisphere, such as in Mediterranean areas. The skilled person will be able to time the administration to be before the onset of the bee season of specific geographical regions.

It is noted such timing of administration is less relevant in relation to greenhouse workers, where the bee season may last all year.

In an embodiment, the single domain antibodies according to the invention, the multispecific antibodies according to the invention and/or the composition or combination according to the invention is for use in a mammal subject, such as livestock or pet or racing animal, such as horses or camels, preferably the subject is a human.

In an embodiment, said subject, preferably a human, suffers from bee venom allergy.

Administration of the compound can be done in a number of ways as described in the following, non-limiting examples. Oral or nasal, which is administration through the mouth or nose. By intradermal injection, which is a delivery of the compound into the dermis of the skin, located between epidermis and the hypodermis. Alternatively, the compound can be administered intravenous, which is an administration directly into the blood stream of the subject. Further, intramuscular administration of the compound is an injection into the muscles of the subject. In addition, the compound can be administered subcutaneous, which is under the skin, in the area between the muscle and the skin of the subject. Further, the compound can be administered intratracheal, which is administration directly into the trachea and by transdermal administration, which is administration across the skin.

Any mode of administration can be used as long as the mode results in the desired effect of the compound.

Thus, in one embodiment, the compound is administered to the subject by oral or nasal administration.

In a preferred embodiment, the compound is administered to a subject by oral administration.

In another preferred embodiment, the compound is administered to a subject by inhalation.

### Nucleic Acid

The different types of single domain antibodies according to the invention may be expressed from a nucleic acid. Thus, an aspect of the invention relates to a nucleic acid encoding the single domain antibodies according to the invention and/or the multispecific antibodies according to the invention.

### Vector

The nucleic acids according to the invention may be located in a vector. Thus, another aspect of the invention relates to a vector comprising the nucleic acid according to the invention, such as an expression vector, such as a plasmid, such as the pET22 vector.

### Host Cell

The vector according to the invention may be located in a host cell. Thus, yet another aspect of the invention relates to a host cell comprising the vector according to the invention.

In an embodiment, the host cell is selected from the group consisting of mammalian cells, yeast cells, insect cells and bacterial cells, preferably mammalian cells.

### In Vitro Uses

The single domain antibodies according to the invention, the multispecific antibodies according to the invention and/or the composition or combination according to the invention may also find use in vitro. Thus, a further aspect of the invention relates to the in vitro use of the single domain antibodies according to the invention, the multispecific antibodies according to the invention and/or the composition or combination according to the invention, such as for standardization, such as for standardizations of diagnostic or therapeutic products, such as honeybee venom products.

### Other Aspects of the Invention

A further aspect of the invention relates to a method for treatment, prevention and/or alleviation of bee allergy in a subject in need thereof, the method comprising administering one or more of the single domain antibodies according to the invention, one or more of the multispecific antibodies according to the invention and/or the composition or combination according to the invention to the subject.

Still a further aspect of the invention relates to a method for treatment, prevention and/or a venom induced toxicity in a subject in need thereof, the method comprising administering one or more of the single domain antibodies according to the invention, one or more of the multispecific antibodies according to the invention and/or the composition or combination according to the invention to the subject.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Production of Nanobodies

### Aim of Study

To select nanobodies specific for the major hymenoptera venom allergen Api m 1 and Api m 2.

### Materials and Methods

### Immunization and Generation of Phage Library:

Llamas were immunized with native Api m1 (nApi m 1) or recombinant Api m 2 (rApi m 2) expressed in Sf9 insect cells. The immunization was performed by Capralogics and comprised four injection with the allergen over a period of 84 days.

Phage libraries were generated by extraction of mRNA from peripheral blood mononuclear cells of immunized llamas. The extracted mRNA was reverse-transcribed into cDNA that was then used for PCR amplification of the VHH domain. The VHH domains were amplified carrying the restriction sites for SfiI and XhoI, for introduced into the phagemid vector; pCANTAB. Ligation of the VHH domain into the vector resulted in the VHH domain being fused to the pIII minor capsid protein from M13 bacteriophages. The phagemid vectors were subsequently transformed into E. coli ER2738 by electroporation.

### Phage Display:

Selection of the phage library was essentially perform as previously described by Griffiths et al. (EMBO J. 1994; 13 (14): 3245-60). and consist of repeated rounds of selection and phage propagation. The method is described in details in the following.

### Propagation of Phages:

The phage library in E. coli ER2738 cells was grown in 2YT media containing 100 μg/ml ampicillin and 1% glucose at 37° C. shaking until an OD600 of 0.4. Phages propagation was initiated by inoculation of a 50 ml culture with 2×10¹¹ KM13 helper phages. After additional 30 min of incubation the media was changed to 2YT containing 100 μg/ml ampicillin, 50 μg/ml kanamycin and 0.1% glucose and the culture was incubated o.n. at 30° C. shaking. Phages were purified by centrifugation at 4000×g for 10 min at 4° C. and subsequently precipitated from the supernatant by incubation with 20% PEG and 2.5 M NaCl for 1 h on ice. After two rounds of precipitation, phages were resuspended in PBS. Titers were determined by infection of E. coli ER2738 with a dilution series of the phages and subsequent plating.

### Selection of Phages:

The phage library was selected using microwells coated with the relevant allergen (nApi m 1 or rApi m 2) in PBS. For the first round of selection, wells were coated with 1 μg of allergen. For the following rounds, wells were coated with 0.1 μg of allergen. After o.n incubation at 4° C. the wells were blocked with PBS containing 3% skimmed milk powder for 1 h and thereafter incubated with phages (2-3×10¹² phages in the first round and 2-3×10¹⁰ phages in the following rounds) for 1 h. After stringent washing, bound phages were eluded with 0.2 M glycine, pH 2.2. The eluted phages were neutralized with 1 M Tris pH 9.1 and used for infection of E. coli ER2738 and titers were determined by plating of dilution series. For subsequent rounds of selections, colonies were scraped from the agar plates, and the propagation and selection was repeated as described above.

### Phage ELISA:

After repeated rounds of selection the polyclonal phages were assessed for binding to the allergen by ELISA. Briefly, microwell plates were coated with 10 μg/ml nApi m 1 or rApi m 2 diluted in PBS at 4° C. o.n. After blocking for 1 h with PBS containing 4% skimmed milk powder, the eluted polyclonal phages were diluted 1:5 and incubated in the wells for 2 h at rt. shaking. After washing, bound phages were detected with HRP conjugated anti-M13 antibody (GE Healthcare). For assessment of monoclonal phages, E. coli ER2738 infected with polyclonal phages were plated and individual colonies were picked and expanded for monoclonal phage propagation. Monoclonal phages were assessed by ELISA as described above.

### Expression and Purification of Nanobodies:

Identified nanobodies were recloned from the pCANTAB vector into pET22 by SLICE cloning using nanobody and vector specific primers. The pET22 vector used for the cloning already contained a pelB signaling sequence for secretion into the periplasmic space, a polyhistidine-tag for purification and a V5-tag or a HA-tag for detection.

The nanobodies were expressed in BL21 Rosetta™ (DE3) cells. The transformation was achieved by heat shock and transformed cells were grown to OD600 0.6 before induction with 0.5 M IPTG and o.n. incubation at 20° C. shaking. Expressed nanobodies were purified from the culture supernatant by Immobilized metal affinity chromatography on the ÄKTA™ start protein purification system using HisTrap™ Excel columns (GE Healthcare). The culture supernatants were run on the column and bound proteins were eluted with 500 mM Imidazole.

### Results

Api m 1 and Api m 2 specific nanobodies were selected by phage display. Two rounds of selections were performed using microwells coated with nApi m 1 or rApi m 2. After the selection, polyclonal phages from each round of the selection were assessed by ELISA. The polyclonal phage ELISA demonstrated significant enrichment of the libraries already after the first round of selection (FIG. 1A-B). Subsequently, monoclonal phages from the second round of selections were assessed and exhibited pronounced reactivity in phage ELISA (FIG. 1C-D). This suggests presence of a significant number of Api m 1 specific clones after the second round of selection.

Sequence analysis of the monoclonal phages resulted in a panel of 12 different Api m 1 specific nanobodies (FIG. 2A) and 5 different Api m 2 specific nanobodies (FIG. 2B). The selected nanobodies are highly divergent in the CDR3 sequence with length ranging from 10 to 19 residues and 4 to 25 residues for Api m 1 and Api m 2 specific nanobodies respectively. This makes it very likely that the identified nanobodies recognize different epitopes on their target allergen.

All selected nanobodies were recloned into pET22 and recombinantly expressed in bacteria. During the cloning a pelB signal sequence, a polyhistidine-tag and a V5-tag or a HA-tag was added to the nanobodies which allowing for secretion into the periplasmic space and subsequent purification and detection. Thus, expressed nanobodies were purified from the culture supernatant by immobilized metal affinity chromatography. FIG. 3 shows an example of an SDS-page of two of the purified Api m 1 specific nanobodies (AM1-1 and AM1-3), confirming the expected molecular masses of around 15 kDa.

### Conclusion

A llama-derived immune-repertoire library and phage display was applied for the generation of nanobodies with specificity for the major insect venom allergens Api m 1 and Api m 2. Two rounds of allergen directed selection, resulted in a panel of 12 Api m 1 specific nanobodies and 5 Api m 2 specific nanobodies that are highly divergent in the CDR3 sequences.

### Example 2—Immunoreactivity of Nanobodies

### Aim of Study

To assess the specificity of the nanobodies selected against Api m 1 and Api m 2.

### Materials and Methods

### Immunoreactivity:

The immunoreactivity of expressed nanobodies was assessed by ELISA. Briefly, micro well plates were coated with 10 μg/ml nApi m 1 or rApi m 2 diluted in PBS at 4° C. o.n. After blocking for 1 h with PBS containing 4% skimmed milk powder, purified nanobodies were incubated in the wells for 2-3 h at rt. shaking. After washing, nanobodies were detected with rabbit anti-V5 antibody (antibodies-online) and AP-conjugated goat anti-rabbit IgG antibody (Sigma) or with mouse anti-penta-His and AP-conjugated rabbit anti-mouse IgG antibody (Sigma).

### Results

The immunoreactivity of the purified nanobodies was analyzed by ELISA (FIG. 4A-B). This confirmed that all purified nanobodies bound their target allergen.

### Conclusion

All the 12 nanobodies selected against Api m 1 binds immobilized nApi m 1 in an ELISA setup.

All the 5 nanobodies selected against Api m 2 binds immobilized rApi m 2 in an ELISA setup.

### Example 3—Characterization of Epitopes

### Aim of Study

To identify Api m 1 specific nanobodies with non-overlapping epitopes. This will allow for the identification of nanobodies that can be used in combination for inhibition purposes.

### Materials and Methods

Affinity Determination and Epitope Binning using Biolayer Interferometry: BLI measurements were performed at 30° C. on an Octet RED96 machine (ForteBio) using amine reactive second-generation (AR2G) biosensors (ForteBio).

Sensors were prepared by dipping the sensors into a solution of nAPi m 1 at 20 μg/ml in 10 mM Na acetate pH 6.0 for 20 min. The reaction was quenched for 300 s in 0.5 M ethanolamine pH 8.5 and the baseline was obtained after 120 s wash in kinetic buffer (PBS, 0.02% Tween20, 0.1% BSA). Association with nanobody was performed over 600 s and the dissociation phase in the same buffer was monitored for 1200 s. Binding in the absence of antigen was used as a reference to subtract the non-specific interactions with the biosensor. Curve fitting was performed with Octet software using 1:1 binding model.

For epitope binding the secondary association of nanobodies to the antigen was assessed in the same manner.

### Results

In order to identify nanobodies with non-overlapping epitopes the binding behavior of individual nanobodies was further assessed using biolayer interferometry. Kinetic profiles of individual nanobodies were generated by measuring the association and dissociation of the nanobodies to sensors loaded with Api m 1. The kinetic profiles indicated large differences in binding kinetics of the various clones.

FIG. 5A-D show examples of four nanobodies with relative high on-rate and low off-rate.

Secondary binding of nanobodies to the already formed allergen/nanobody complex on the surface further suggested that some nanobodies recognize non-overlapping epitopes.

As shown in FIG. 5E-G association of the nanobodies AM1-3, Am1-4 and Am1-6 after the association of AM1-1 can be observed indicating that AM1-1 binds a different epitope than the three other nanobodies.

In contrast, Am1-3 and AM1-6 cannot associate to sensors with AM1-4 already associated indication that AM1-3, AM1-4 and AM1-6 binds to overlapping epitopes (FIG. 5H-I).

A full analysis indicated that the nanobody AM1-1 recognizes an epitope that is different from any of the other nanobodies whereas all other nanobodies seems to bind overlapping epitopes.

### Conclusion

Using biolayer interferometry (BLI) it is shown that the nanobody AM1-1 recognize an epitope that is different from any of the other nanobodies whereas all other nanobodies seems to bind overlapping epitopes. This suggest that AM1-1 can be used in combinations with other nanobodies for simultaneous binding and presumed inhibition of the allergen.

### Example 4—Generation of Nanobody-hIgE Formats

### Aim of Study

To establish a nanobody-based IgE format that can be used as a surrogate of human IgE in research and diagnostics.

### Materials and Methods

### Generation of Nanobody-hIgE Formats:

A nanobody human IgE format (nb-hIgE) was generated by fusion of selected nanobodies to the human IgE fc domains.

Nanobodies were amplified from the pCANTAB vectors by PCR and the product was cloned into a pCDNA3.1 vector between a human immunoglobulin-derived signal sequence and the human IgE Fc domains CH2-CH4.

### Expression and Purification of Nanobody-hIgEs:

Nb-hIgEs were expressed in mammalian HEK293 or FreeStyle 293-f cells. Transfection was performed with polyethylenimine (PEI) in a 1:3 ratio of DNA and PEI and stably transfected cells were selected by adding zeocin to the media. Expressed nb-hIgEs were purified from the supernatant of stable transfected cells by immobilized metal affinity chromatography in the same way as described for the nanobodies.

### Immunoreactivity:

Immunoreactivity of purified nb-hIgEs were assessed by ELISA as described for the nanobodies but using AP-conjugated rabbit anti-hIgE antibody (Sigma) for detection.

Immunoreactivity and cross reactivity was also assessed using the commercial Euroline assay DPA-Dx Insect venoms 2 (Euroimmun) according to the manufacturer's instructions.

### Results

Selected nanobodies specific for Api m 1 and Api m 2, were converted into homodimeric IgE formats by fusion of the nanobodies to the human IgE CH2-CH4 domains.

The nanobody based human IgE formats (nb-hIgE) were expressed in mammalian HEK cells and purified by immobilized metal affinity chromatography. The expressed nb-hIgEs were analysed by SDS-page and immunoblotting (FIG. 6A-B). This verified the expected molecular mass of approximately 100 kDa and suggested proper folding, dimerization and glycosylation of the nanobody IgE format.

The immunoreactivity of the purified nb-IgEs were assessed by ELISA (FIG. 7A-B). This demonstrated binding to their target allergens (Api m 1 and Api m 2), as well as to the FcεRIα receptor.

Moreover, the nb-IgEs exhibited reactivity to both venom extract and the individual allergen without any cross-reactivity to other allergens upon application to a commercial Euroline tests containing the venom extracts as well as a number of individual venom allergens (FIG. 7C).

### Conclusion

Nanobody-based human IgE formats that bind specifically to the allergens Api m 1 and Api m 2 were successfully generated.

### Example 5—Functional Activity of Nb-IgEs

### Aim of Study

To assess the functionality of the nanobody-based IgE format in order to investigate if the formats can be used as surrogates of human IgE in research and diagnostics.

### Materials and Methods

### Functionality Assays:

The functionality of the nb-hIgEs were assessed by activation of effector cells (RBL-SX38) in a β-Hexosaminidase release assay as previously described (Hecker et al. Mol Immunol. 48(9-10):1236-44) RBL-SX38 cells expressing the human FcεRIα receptor were sensitized with 1 μg/ml nb-IgEs for 1 h at 37° C. After washing, the cells were incubated with dilution series of the relevant allergen and incubated for 1 h at 37° C. As a reference, cross-linking was achieved by incubation with polyclonal anti-human IgE antibody (Dako). β-Hexosaminidase release of viable versus lysed cells was assessed with p-nitrophenyl N-acetyl-glucosaminide (Sigma) as a substrate.

In order to evaluate the capability of nb-hIgEs to form IgE/allergen complexes and bind to the low affinity receptor CD23 an ELIFAB assay was performed as previously described by Shamji et al. (J Allergy Clin Immunol. 2013; 132(4):1003-5 e1-4). Nb-hIgEs (5 μg/mL) were pre-incubated with antigen (0.33 μg/mL) at 37° C. for 1 h for formation of IgE/allergen complexes. After pre-incubation, nb-hIgE/allergen complexes were transferred to micro well plates coated with soluble CD23 (R&D Systems) and incubated for 1 h at room temperature. After washing, nb-hIgE/allergen complexes bound to CD23 were detected by biotin-conjugated anti-human IgE antibody (BD Biosciences) and AP-conjugated ExtrAvidin (Sigma).

### ImmunoCAP:

The sIgE and total IgE antibody concentrations were measured using immunoCAPs (Thermo Fischer Scientific) at Aarhus University Hospital.

### Results

In order to assess the functional activity of the nb-IgEs, degranulation of nb-IgE loaded RBL-SX38 cells upon allergen stimulation was analyzed. Anti-IgE mediated activation could be shown for all nb-IgEs (data not shown) confirming the loading of the IgEs to cells. As allergen-dependent activation requires cross-linking of two IgE molecules with non-overlapping epitopes, combinations of nb-IgE was analyzed (FIG. 8A-B). For the two Api m 2 specific nb-IgEs, AM2-A1 and AM2-C2, allergen-dependent activation could be clearly shown for the combinations of nb-IgEs compared to single nb-IgEs. For the two Api m 1 specific nb-IgEs, Am1-1 and AM1-3, a clear allergen-dependent activation was also observed. Notably, for one of the nb-IgEs, AM1-3, allergen-mediated cellular activation was observed without addition of the complementary nb-IgE.

In order to further evaluate the functional activity of the nb-IgEs, an ELIFAB assay was performed to evaluate formation of allergen/IgE immune complexes and subsequent binding to the CD23 receptor (FIG. 9A-B). This assay revealed pronounced binding of Api m 1 and Api m 2/IgE complexes to surface-immobilized CD23. Omission of one of the IgE as well as omission of antigen decreased the binding.

In order to investigate whether the established nb-IgEs can be used of standardization purposes in diagnostic tests, the nb-IgEs were subjected to sIgE determination using the immunoCAP test system. Pronounced, concentration-dependent IgE reactivity was observed for all nb-IgEs both when testing against venom extract and specific allergen (FIG. 10). This supported their functionality as well as the quality of the nb-hIgEs for use in diagnostic test systems. For Api m 1 specific nb-IgEs, the sIgE reactivity against rApi m 1 was highly similar to that against HBV. In contract, a significant reduction in reactivity against venom extract as compared to recombinant allergen was observed for the two Api m 2 specific nb-IgEs. This emphasizes the superiority of component testing in HVA diagnostics.

### Conclusion

Selected nanobodies were used for the generation of a novel nanobody-based human IgE format. Our data show that the nanobody-based IgE format efficiently mimics the functional activity of IgE with regard to receptor binding and cellular activation.

Importantly, as allergen-dependent activation requires cross-linking of two IgE molecules with non-overlapping epitopes the cellular assay support that the nanobodies AM1-1 and AM1-3 as well as the nanobodies AM2-A1 and AM2-C2 bind non-overlapping epitopes. This is also supported by the ELIFAB assay. In this assay, we observed an increased binding when using the nb-IgEs in combination as compared to the individual nb-IgEs.

This indicates that the nb-IgEs bind different epitopes and thereby can form larger complexes, which increases the binding to the low-affinity receptor CD23.

In addition, proof-of-concept for the use of nanobody-based IgE for standardization in a diagnostic setup was shown. The efficacy and ease in generation of the nanobody-based IgEs makes the format very interesting as a tool for research and diagnostic testing.

### Example 6—Bispecific Nanobody-Based IgG1

### Aim of Study

To establish a nanobody-based bispecific IgG1 format.

### Materials and Methods

### Generation of Bispecific Nanobody Based IgG₁:

Bispecific IgG₁ formats were generated by introducing DEKK mutations into human IgG₁ fc as described by De Nardis et al. (J Biol Chem. 2017; 292(35): 14706-17). Briefly, one pCDNA3.1 vector containing the human IgG₁ fc with L351D and L368E mutations and a BC2-tag was constructed. Another pCDNA3.1 vector containing the human IgG₁ fc with L351K and T366K mutations and a polyhistidine-tag was also constructed. This introduction of so-called “DEKK mutations” facilitates the formation of heterodimeric IgG₁. In addition, one vector contains the gene for Zeocin resistance while the other contains the gene for Geneticin (G418) resistance. This makes it possible to select for cells successfully transfected with both vectors.

Selected nanobodies (AM1-1 and Am1-3) were amplified from the pCANTAB vectors by PCR and the product was cloned into one of the two pCDNA3.1 vectors between a human immunoglobulin-derived signal sequence and the hinge region of the mutated human IgG₁ Fc domains.

The bispecific nanobody based human IgG₁ were expressed in mammalian HEK293 cells. Transfection was performed by co-transfection of the cells with the two different IgG₁ construct in a 1:1 ratio. The transfection was performed with a 1:3 ratio of DNA and PEI and stably transfected cells were selected by adding Zeocin and Geneticin (G418) to the media.

Expressed antibodies were purified from the supernatant of stable transfected cells by immobilized metal affinity chromatography in the same way as described for the nanobodies.

Formation of bispecific antibodies was confirmed by ELISA. Briefly, microwells were coated with the bispecific antibodies and detected by a biotinylated anti-BC2 antibody (Chromotek).

### Results

A bispecific nanobody based IgG₁ format comprising the two nanobodies Am1-1 and AM1-3 was generated by fusion of the nanobodies to the hinge region of human IgG₁ Fc domains containing DEKK mutations for facilitated formation of heteromeric antibodies.

The bispecific nanobody-based IgG₁ format was expressed in mammalian HEK cells and purified from the medium by immobilized metal affinity chromatography via a polyhistidine-tag only present on one of the heavy chains. The purified antibodies were analyzed by SDS-page verifying the expected molecular mass of approximately 80 kDa and suggesting proper folding and dimerization (FIG. 11A).

Formation of bispecific antibodies was confirmed by ELISA (FIG. 11B). Immobilized bispecific antibody was detected using an anti-BC2 antibody. As the BC2-tag and the polyhistidine-tag are not both present on the same heavy chains, purification by the polyhistidine-tag and subsequent detection by the BC2-tag confirms the formation of heterodimeric bispecific antibodies.

### Conclusion

A bispecific nanobody IgG₁ format comprising two different Api m 1 specific nanobodies was successfully generated.

### Example 7—Nanobody-Based Inhibition

### Aim of Study

To assess the ability of nanobodies and nanobody-formats to inhibit the binding of patient IgE to immobilized Api m 1.

### Materials and Methods

A 384 micro well plate was coated with 5 μg/ml nApi m 1 diluted in PBS at 4° C. o.n. After blocking for 30 min with PBS containing 4% skimmed milk powder, the wells were incubated with 3 μM purified nanobody or bispecific nanobody based human IgG₁ for 1 h at rt. shaking. Serum from a bee venom allergic patient was added on top of the nanobodies, and the plate was incubated for additional 1 h. As a positive control, negative serum was spiked with 20 kU/L Am1-1hIgE and AM1-3 hIgE and incubated in the wells instead of patient serum. After washing, bond IgE was detected using AP-conjugated rabbit anti-hIgE antibody (Sigma).

### Results

To assess the ability of the selected nanobodies to inhibit the binding of patient's IgE to Api m 1 an inhibition ELISA was developed. Immobilized Api m 1 was incubated with a large excess of purified nanobodies or bispecific nanobody based IgG₁ followed by incubation with bee venom allergic patient serum containing Api m 1 specific IgEs. As an initial positive validation of the assay, negative serum spiked with AM1-1 hIgE and AM1-3 hIgE was analyzed (FIG. 12A). In this assay, incubation with the nanobodies AM1-1 or AM1-3 resulted in a reduced binding of IgE of 30% and 40% respectively. Mixing the two nanobodies had an additive effect and resulted in a 70% reduction. A similar reduction was observed when incubating with the bispecific nanobody based IgG₁ comprising the same two nanobodies.

After this initial validation of the assay, a bee allergic patient serum was analyzed in the assay (FIG. 12B). Here, only minor inhibition was observed when incubating with the nanobody AM1-1 whereas no inhibition was observed when incubating whit the nanobody AM1-3 or a mix of the two. However, when incubating with a bispecific IgG₁ comprising the same two nanobodies a large reduction of 60% was observed. It is not evident from this assay whether the increased inhibition by the bispecific-format is a result of higher affinity of the bivalent antibody or simply a matter of increased steric hindrance by the larger molecule.

### Conclusion

This initial test of nanobody-based inhibition clearly demonstrates a potential of using nanobody-based formats for blocking the binding of patient IgE to Api m 1, in particular in the bispecific format.

### Example 8—Generation of Nanobody-Based Human IgG₁

### Aim of Study

To generate nanobody-based monospecific and bispecific IgG₁.

### Materials and Methods

### Generation of Nanobody-Based Human IgG₁:

For generation of monospecific nanobody-based human IgG₁ (Nb-hIgG₁), selected nanobodies (AM1-1 and AM1-4) were amplified from the pCANTAB vectors by PCR and the product was cloned into pCDNA3.1 vectors between a human immunoglobulin-derived signal sequence and the hinge region of a human IgG₁ Fc domain.

Bispecific IgG₁ formats were generated by introducing DEKK mutations into human IgG₁ fc as described by De Nardis et al. (1). Briefly, one pCDNA3.1 vector containing the human IgG₁ fc with L351D and L368E mutations and a BC2-tag was constructed. Another pCDNA3.1 vector containing the human IgG₁ fc with L351K and T366K mutations and a polyhistidine-tag was also constructed. This introduction of so-called DEKK mutations facilitates the formation of heterodimeric IgG₁. In addition, one vector contains the gen for Zeocin resistance while the other contains the gen for Geneticin (G418) resistance. This makes it possible to select for cells successfully transfected with both vectors.

### Expression and Purification of Nanobody-Based IgG₁:

The monospecific nanobody-based IgG₁ were transiently expressed in mammalian FreeStyle™ 293-F cells. The transfection was performed with a 1:2 ratio of DNA and PEI.

The bispecific nanobody-based human IgG₁ were expressed in mammalian HEK293 cells. Transfection was performed by co-transfection of the cells with the two different IgG₁ construct in a 1:1 ratio. The transfection was performed with a 1:3 ratio of DNA and PEI and stably transfected cells were selected by adding Zeocin and Geneticin (G418) to the media.

Expressed antibodies were purified from the supernatant of transient or stable transfected cells by immobilized metal affinity chromatography on the ÄKTA™ start protein purification system using a HisTrap™ Excel columns (GE Healthcare). The culture supernatants were run on the column and bound proteins were eluted with 500 mM Imidazole. Then nanobody-based human IgG₁ were further purified by size exclusion chromatography using a HiLoad 16/600 200 pg superdex column (GE Healthcare) equilibrated in PBS pH 7.4.

### Immunoreactivity:

The immunoreactivity of recombinantly expressed nanobody-based human IgG₁s was assessed by ELISA. Briefly, micro well plates were coated o.n. at 4° C. with 2 μg/ml nApi m 1 diluted in PBS. After blocking for 30 min with PBS containing 4% skimmed milk powder, 100 nM purified nanobody-based human IgG₁ was incubated in the wells for 3 h at rt. shaking. After washing, bound nanobody-based human IgG₁ was detected with alkaline phosphatase-conjugated anti-hIgG antibody (Sigma).

### Results

Monospecific nanobody-based human IgG₁ (AM1-1 IgG₁ and AM1-4 IgG₁) comprising either the nanobody AM1-1 or the nanobody AM1-4 were generated by fusion of the nanobodies to the hinge region of human IgG₁ Fc domains. In addition, a bispecific nanobody-based human IgG₁ format (AM1B-1-4 IgG₁) comprising both of the nanobodies was generated by fusion of the nanobodies to the hinge region of human IgG₁ Fc domains containing DEKK mutations for facilitated formation of heterodimeric proteins.

The nanobody-based IgG₁ formats were expressed in mammalian HEK cells and purified from the supernatant by immobilized metal affinity chromatography and size exclusion chromatography. The purified antibodies were analyzed by SDS-page and western blot under reducing and non-reducing condition. The nanobody-based human IgG₁ formats are expected to have a molecular mass of approximately 80 kDa. At non-reducing conditions, bands are observed at approximately 100 kDa. The higher molecular mass is most likely caused by glycosylation of the constructs. At reducing condition, bands are observed at approximately half the molecular mass, suggesting proper folding and dimerization of the nanobody-based human IgG₁ formats (FIG. 13). The immunoreactivity of the purified nanobody-based human IgG₁ formats was assessed by ELISA. The obtained data demonstrated binding of all three nanobody-based human IgG₁s to their target allergen Api m 1 (FIG. 14).

### Conclusion

We successfully generated monospecific and bispecific nanobody-based human IgG₁ formats that bind specifically to the allergen Api m 1.

### Example 9—Binding Kinetics of Nanobody-Based Human IgG₁ Formats

### Aim of Study

To analyse the affinity of the monospecific and bispecific nanobody-based human IgG₁.

### Materials and Methods

### Affinity Determination Using Biolayer Interferometry:

Biolayer interferometry (BLI) measurements were performed at 30° C. on an Octet RED96 system (ForteBio) using Anti-Human Fc Capture (AHC) Biosensors (Sartorius). All samples were prepared in Kinetic buffer (PBS pH 7.4, 0.1% BSA and 0.02% Tween20). Sensors were loaded with 2 nM nanobody-based human IgG₁ for 420 s and a baseline was obtained after 300 s in Kinetic buffer. Association was performed over 300 s with 2-fold dilution series of nApi m 1 from 160 nm to 5 nM (AM1-1 hIgG₁), 40 nM to 1.25 nM (AM1-4 hIgG₁) and 50 nm to 0.8 nM (AM1B-1-4 hIgG₁). Dissociation was measured for 1200 s in Kinetic buffer. Binding in the absence of allergen was used as a reference to subtract the non-specific interactions with the biosensor. Curve fitting was performed with Octet software using 1:1 binding model.

### Results

The two monospecific nanobody-based IgG₁ bound nApi m 1 with high affinities of 1.94·10⁻⁹ M and 3.00·10⁻¹⁰ M for AM1-1 hIgG1 and AM1-4 hIgG1 respectively (FIG. 15).

As expected, the binding of the bispecific nanobody-based human IgG₁ format, AM1B-1-4, was enhanced compared to the monospecific formats indicating an increased avidity mediated by the binding of two distinct Api m 1 epitopes.

### Conclusion

Using biolayer interferometry, we showed that the nanobody-based human IgG₁ formats bind Api m 1 with affinities in the nanomolar to subnanomolar range and a clear benefit for simultaneous targeting of two epitopes.

### Example 10—Inhibition with Nanobody-Based Human IgG1

### Aim of Study

To assess the ability of nanobody-based human IgG₁ formats to block the binding of Api m 1-specific patient IgE to immobilized Api m 1.

### Materials and Methods

A high binding 384 micro well plate (Greiner Bio-One) was coated o.n. at 4° C. with 5 μg/ml nApi m 1 (Latoxan) diluted in PBS. After blocking for 30 min with TBS containing 4% BSA, the wells were incubated for 1 h at rt. shaking with 1 UM nanobody-based human IgG₁ diluted in TBS+2% BSA. Serum from honeybee venom allergic patient sensitized to Api m 1 was then added on top of the nanobody-based human IgG₁s, and the plate was incubated for additional 1 h. After washing, bound IgE was detected by 1 h incubation with AP-conjugated anti-hIgE antibody (Sigma) diluted in TBS+2% BSA. The plate was developed with substrate solution (5 mg/mL 4-nitro-phenylphosphate, Sigma) for 1 h and 40 min and the plate was measures at 405 nm.

### Results

The ability of the nanobody-based human IgG1 formats to inhibit the binding of Api m 1-specific IgE from allergic patients to the allergen Api m 1, was assessed by an inhibition ELISA. Immobilized Api m 1 was incubated with a large excess of nanobody-based human IgG1 followed by incubation with honeybee venom allergic patient serum containing Api m 1-specific IgEs. In total, sera from five different honeybee venom allergic patients was tested.

In the inhibition ELISA, each of the monospecific nanobody-based human IgG1 partially blocked the binding of IgE to Api m 1 with a median inhibition of 49% and 62% for AM1-1 IgG1 and AM1-4 IgG1 respectively.

An increased median inhibition of 89% was observed when combing the two monospecific nanobody-based and a median inhibition of 93% was observed when applying the bispecific nanobody-based IgG1.

### Conclusion

In this study, we observed a median inhibition of 93% of the binding of patient IgE to the allergen Api m 1 when inhibiting with the bispecific nanobody-based human IgG₁ format in contrast to a clearly reduced inhibition when using single nanobody-based human IgG₁ format alone. This clearly demonstrates the potential of using nanobody-based formats for blocking the binding of patient IgE to Api m 1.

### Example 11—Structure of Api m 1 in Complex with Nanobodies AM1-1 and AM1-4

### Aim of Study

We aimed to obtain a crystal structure of Api m 1 in complex with the two nanobodies AM1-1 and AM1-4 in order to analyze the nanobody-allergen interactions.

### Materials and Methods

### Crystallization:

nApi m 1, AM1-1 and AM1-4 were concentrated to 14.5 mg/ml in 20 mM HEPES, 150 mM NaCl 10 mM CaCl₂), pH 7.4 and mixed 1:1:1. The complex was crystallized by vapor diffusion in sitting drops formed by mixing protein solution and reservoir solution containing 0.2 M NaCl, 0.1 M Phosphate/citrate pH 4.2 and 20% w/v polyethylene glycol (PEG) 8000 in 1:1 ratio. Crystals were cryocooled in liquid nitrogen.

### Data Collection and Structure Determination and Refinement:

Data were collected at the beamline P13 operated by EMBL at the PETRA III storage ring (DESY, Hamburg, Germany) and processed with XDS (1). The structure was determined by molecular replacement with PHASER (2), using pdb ID: 1POC as search model for Api m 1. Manual model building was done in COOT (3). The final model was obtained from several cycles of rebuilding in COOT and refinement with PHENIX refine. Figures were prepared with the PyMOL Molecular Graphics System (Schrödinger LLC). Interactions between nApi m 1 and the nanobodies were analysed by PDBePISA.

### Results

In order to understand the nanobody-allergen interactions, we examined the complex of Api m 1 bound by the two nanobodies AM1-1 and AM1-4 using X-ray crystallography. We obtained a crystal that diffracted X-rays to a maximum resolution of 1.8 Å (Table 1).

TABLE 1
Data collection

| | |
|---|---|
| Wavelength | 0.97626 |
| Space group | C121 |
| Cell dimensions | |
| a, b, c (Å) | 133.96, 51.33, 73.25 |
| α, β, γ (°) | 90.00, 120.78, 90.00 |
| Resolution (Å) | 42.03 - 1.76 |
| Completeness (%) | 98.3 |
| Mean I/sigma(I) | 12.48 |
| Total number of reflections | 186541 |
| Number of unique reflections | 42084 |

The structure revealed a 1:1:1 complex in which the two nanobodies binds epitopes located on opposite sides of the Api m 1 molecule (FIG. 17A). The epitopes of the two nanobodies does not overlap, which is consistent with our BLI data. As expected, the CDRs of the nanobodies are responsible for the majority of the interaction. The relative large CDR3 of AM1-4 (19 residues) is reaching into a pocket located between the α-helical segment and the double-stranded antiparallel β-sheets (FIG. 17B). On the opposite side of the Api m 1 molecule, the CDR3 of AM1-1 interact with the active site of Api m 1 (FIG. 17B). It is therefore most likely that the nanobody AM1-1 interferes with the enzymatic activity of Api m 1.

Pisa analyses (proteins, interfaces, structures and assemblies), were used to further describe the interface between Api m 1 and the two nanobodies (Table 2). The recognition is primarily driven by CDR2 and CDR3 as expected.

Table 2: Complex between PLA2 and the two nanobodies: The residues at the interface between PLA2 (chain A) and Nb1-4 (chain B) are 25 for PLA2 and 20 for Nb1-4. The residues at the interface between PLA2 and Nb 1-1 (chain C) are 29 for PLA2 and 20 for Nb1-1. The ones involved in interactions are listed below. Buried surface area (BSA) between chain A and B is 1470.5 Å² (681.1 Å² in A and 789.4 Å² in B). BSA between chain A and C is 1592.3 Å² (728.9 Å² in A and 863.4 Å² in C). The BSA for the entire assembly is 3062.8 Å².

| Api m 1 | NB | | | |
|---|---|---|---|---|
| RESIDUE | (Chain B | | | |
| (Chain A) | or C) | RESIDUE | Location | BOND |
| Asp 65 | NB 1-4 | Gly 103 | CDR3 | H |
| Asp 65 | NB 1-4 | Ser 104 | CDR3 | H |
| Lys 97 | NB 1-4 | Ser 105 | CDR3 | H |
| Glu 99 | NB 1-4 | Arg 107 | CDR3 | H, SB |
| Glu 107 | NB 1-4 | Ser 52 | CDR2 | H |
| Glu 107 | NB 1-4 | Ser 54 | CDR2 | H |
| Cys 113 | NB 1-4 | Ser 105 | CDR3 | H |
| Leu 114 | NB 1-4 | Thr 106 | CDR3 | H |
| His 115 | NB 1-4 | Thr 106 | CDR3 | H |
| Tyr 116 | NB 1-4 | Thr 106 | CDR3 | H |
| Asp 130 | NB 1-4 | Ser 105 | CDR3 | H |
| Arg 132 | NB 1-4 | Glu 99 | CDR3 | H, SB |
| Arg 132 | NB 1-4 | Gly 103 | CDR3 | H |
| Arg 132 | NB 1-4 | Ser 104 | CDR3 | H |
| Arg 132 | NB 1-4 | Thr 106 | CDR3 | H |
| Cys 31 | NB 1-1 | Ser 101 | CDR3 | H |
| His 34 | NB 1-1 | Ser 101 | CDR3 | H |
| Asp 35 | NB 1-1 | Ser 101 | CDR3 | H |
| Ala 43 | NB 1-1 | Tyr 37 | FR2 | H |
| Glu 45 | NB 1-1 | Thr 50 | FR2 | H |
| Glu 45 | NB 1-1 | Asn 58 | CDR2 | H |
| Ser 46 | NB 1-1 | Asn 58 | CDR2 | H |
| Tyr 87 | NB 1-1 | Arg 103 | CDR3 | H |

H: hydrogen bond
SB: salt bridge

Notably, the two nanobodies recognize conformational epitopes on the Api m 1 (FIG. 18). The epitope of the AM1-1 antibodies includes the catalytically relevant residues H34 and Y87, hence an impact on the catalytic activity of the PLA2 can be expected.

### Conclusion

The inventing team have obtained a crystal structure of Api m 1 in complex with the nanobodies AM1-1 and AM1-4 with a resolution of 1.8 Å. The structure shows that the two nanobodies bind mainly with their CDR regions to distinct epitopes on opposite sides of the Api m 1 molecule and that the nanobody AM1-1 is interacting directly with the active site of Api m1, the residues H34 and Y87.

### Example 12—Inhibition of the Enzymatic Activity of Phospholipase A2 (Api m 1)

### Aim of Study

In Example 11, the inventors showed that the nanobody AM1-1 interacts directly with the residues H34, D35 and Y87 of Api m 1. These residues are a part of the active site of Api m 1 and it is therefore reasonable to suggest that at least AM1-1 can inhibit the enzymatic activity of Api m 1. In this study, the inventors investigated the inhibition of the enzymatic activity of Api m 1 mediated by the nanobodies AM1-1 and AM1-4.

### Material and Methods

Inhibition of the enzymatic activity of nApi m 1 (Latoxan) was evaluated with the EnzChek Phospolipase A2 Assay kit (Invitrogen) according to the recommendation of the manufacturer. Briefly, 2 μg/ml nApi m 1 was incubated with the nanobodies (2-fold dilution series starting at 18 μg/ml) and incubated 1 h at room temperature shaking. The enzymatic reaction was started by adding PLA2 substrate-lipid mix to each of the samples. After 10 min incubation at room temperature, fluorescence (Excitation 467±7 and Emission 515±10 and 575±10) was measured with a ClarioStar Plus plate reader. The fluorescence ratio (515 nm/575 nm) was determined, background (no Api m 1) was subtracted and the data was normalized to a positive control (Api m 1 without nanobody).

### Results

In order to assess the inhibition of the enzymatic activity of Api m 1 mediated by the nanobodies AM1-1 and AM1-4, the inventors determined the enzymatic activity of Api m 1 using a fluorescence phospholipase assay in the presence of the nanobodies (FIG. 19). The assay demonstrated that the nanobody AM1-1 can reduce the enzymatic activity of Api m 1 to 50%. The nanobody AM1-4 had no inhibitory effect on the enzymatic activity.

### Conclusion

In this study, the inventors demonstrated that the nanobody AM1-1 can reduce the enzymatic activity of Api m 1 to 50%. In addition to blocking IgE binding to the allergen, the nanobody AM1-1 might therefore also reduce reactions associated with the enzymatic activity of Api m 1.

### Example 13—Inhibition of Basophil Activation

### Aim of Study

In this study, the inventors assessed the ability of the nanobody-based human IgG₁ formats to inhibit the activation of basophils from honeybee venom allergic patients upon allergen stimulation.

### Materials and Methods

Unless otherwise stated, all buffers and reagents used for Basophil Activation Test (BAT) are from the Flow Cast kit (Bühlmann). To evaluate the inhibition of basophil activation, nApi m 1 (Latoxan) or honeybee venom extract (Bühlmann) was pre-blocked with nanobody-based human IgG₁ formats (10-fold serial dilutions) in stimulation buffer for 1 h at room temperature. Peripheral EDTA blood from honeybee venom allergic patients was then incubated with the pre-blocked allergen or extract. Samples were stained with anti-CD63-FITC and anti-CCR3-PE in a 37° C. water bath for 15 min. Erythrocytes were lysed for 7.5 min followed by centrifugation at 600×g for 5 min. Cell pellets were resuspended in 100 μl washing buffer and basophil activation was evaluated by flow cytometry. Basophils were defined as CCR3^{pos}/SSC^{low} cells and activated basophils were distinguished by being CD63 positive.

### Results

In order to assess the inhibitory potential of the nanobody-based human IgG₁ formats, basophils from honeybee venom allergic patents were stimulated with Api m 1 pre-blocked with the nb-hIgG₁. With basophils from one patient (Patient 1), the AM1-1 IgG inhibited 16% of the activation while AM1-4 IgG inhibited 44% of the activation. When inhibiting with a mix of the two nb-hIgG or the bispecific nb-hIgG an additive or even slightly synergetic effect was observed with inhibitions of 69% and 63% respectively (FIG. 20A).

With basophils from another patient (Patient 2), the AM1-1 IgG inhibited 7% of the activation while AM1-4 IgG inhibited 23% of the activation. When inhibiting with a mix of the two nb-hIgG or the bispecific nb-hIgG an additive or even slightly synergetic effect was observed with inhibitions of 38% and 27% respectively (FIG. 20A).

For a third patient (Patient 3), inhibition of basophil activation was assessed both using Api m 1 and honeybee venom extract for the stimulation. With basophils from this patient, the inventors observed a large inhibition with AM1-4 IgG of approximately 90% and an inhibition with AM1-1 IgG of approximately 30%. Almost complete inhibition was observed with the mix of the two nb-hIgG and with the bispecific nb-hIgG. More importantly, the inhibition was not only observed when stimulating with Api m 1 but also when stimulation with honeybee venom extract (FIG. 20B).

### Conclusion

In this study, the inventors assessed the potential of the nanobody-based hIgG₁ to inhibit the activation of basophils from honeybee venom allergic patients. In the study, the inventors demonstrated a clear potential in using nanobody-based formats for inhibiting basophil activation. When testing single nanobody-based IgG, AM1-4 IgG showed the largest inhibition. When combining the nanobody-based hIgG either as a mix or as a bispecific molecule, an additive or slightly synergetic effect was observed. Among the patients, the inventors observed a varying degree of inhibition with the nanobody-based hIgG. This point towards a broad and diverse epitope recognizing by the patient IgE. Therefore, including more nanobodies with distinct epitopes might increase the inhibition of the basophil activation.

### Example 14—Generation of Nanobodies Binding Distinct Epitopes from the Nanobody AM1-4

### Aim of Study

In the previous examples, the inventors showed a large inhibition of the binding of Api m 1 to specific IgE from honeybee venom allergic patients mediated by the nanobody AM1-4. In this study, the inventors designed a selection strategy in order to select new, complementary nanobodies against Api m 1. These nanobodies should bind distinct epitopes from AM1-4 and thereby enable the inventors to combine them with AM1-4 and potentially AM1-1 for further inhibitory studies.

### Materials and Methods

### Selection of Allergen Specific Nanobodies

### Phage Display

In order to select Api m 1 specific nanobodies that bind a distinct epitope than AM1-4, the inventors designed a phage display selecting strategy in which nanobodies were selected against immobilized AM1-4 IgG/Api m 1 complexes. Selection of the phage library was essentially performed as previously described by Griffiths et al. and consists of repeated rounds of selection and phage propagation. The method is described in details in the following.

### Propagation of Phages

The phage library in E. coli ER2738 cells was grown in 2YT media containing 100 μg/ml ampicillin and 1% glucose at 37° C. shaking until an OD600 of 0.4. Phage propagation was initiated by inoculation of a 50 ml culture with 2×10¹¹ KM13 helper phages. After additional 30 min of incubation, the media was changed to 2YT containing 100 μg/ml ampicillin, 50 μg/ml kanamycin and 0.1% glucose and the culture was incubated o.n. at 30° C. shaking. Phages were purified by centrifugation at 4000×g for 10 min at 4° C. and subsequently precipitated from the supernatant by incubation with 20% PEG and 2.5 M NaCl for 1 h on ice. After two rounds of precipitation, phages were resuspended in PBS. Titers were determined by infection of E. coli ER2738 with a dilution series of the phages and subsequent plating.

### Selection of Phages

In the first round of selection, micro wells were coated with 10 μg/ml of AM1-4 IgG in PBS. After o.n incubation at 4° C. the wells were blocked with PBS containing 4% skimmed milk powder for 30 min and thereafter incubated with 2 μg/ml nApi m 1 for 2 h at room temperature. After formation of AM1-4 IgG/Api m 1 complexes, the wells were washed and incubated with 2-3×10¹² phages for 1 h. After stringent washing, bound phages were eluded with 0.2 M glycine, pH 2.2. The eluted phages were neutralized with 1 M Tris pH 9.1 and used for infection of E. coli ER2738 and titers were determined by plating of dilution series.

For the second round of selection, wells were coated with either 1 μg/ml Api m 1 or 1 μg/ml AM1-4 IgG followed by incubation with 2 μg/ml Api m 1 as above. Before the selection against Api m 1 or the AM1-4IgG/Api m 1 complexes, the phages (2-3×10¹⁰) were pre-incubated for 1 h in wells coated with AM1-4 IgG in order to remove any IgG binders. The rest of the selection was performed as above.

### Phage ELISA

After two rounds of selection the polyclonal phages were assessed for binding to Api m 1 by ELISA. Briefly, micro well plates were coated with 2 μg/ml Api m 1 or 10 μg/ml AM1-4IgG diluted in PBS at 4° C. o.n. After blocking for 30 min with TBS containing 4% skimmed milk powder, the wells were incubated with 2 μg/ml Api m 1 for 1 h at room temperature. After formation of AM1-4 IgG/Api m 1 complexes, polyclonal phages (1×10¹² phages/ml) were incubated in the wells for 2 h at room temperature. After washing, bound phages were detected with biotinylated anti-M13 antibody (Invitrogen) and AP-conjugated ExtrAvidin. For assessment of monoclonal phages, E. coli ER2738 infected with polyclonal phages were plated and individual colonies were picked and expanded for monoclonal phage propagation. Monoclonal phages were assessed by ELISA as described above.

### Expression and Purification of Nanobodies

Identified nanobodies were recloned from the pCANTAB vector into pET22b (+) by SLICE cloning using nanobody and vector specific primers. The pET22 vector used for the cloning already contained a pelB signaling sequence for secretion into the periplasmic space, a polyhistidine-tag for purification and a V5-tag for detection. The nanobodies were expressed in BL21 Rosetta™ (DE3) cells. The transformation was achieved by heat shock and transformed cells were grown to OD600 0.8 before induction with 0.5 M IPTG and o.n. incubation at 25° C. shaking. Expressed nanobodies were purified from the culture supernatant by Immobilized metal affinity chromatography on the ÄKTA™ start protein purification system using HisTrap™ Excel columns (GE Healthcare). The culture supernatants were run on the column and bound proteins were eluted with 500 mM Imidazole.

### Results

New Api m 1 specific nanobodies were selected by phage display. Two rounds of selection were performed. In the first round, the selection was performed against immobilized AM1-4 IgG/Api m 1 complexes. In the second round, the selection was performed against either immobilized Api m 1 or AM1-4 IgG/Api m 1 complexes. After the selection, polyclonal phages from each round of the selection were assessed by ELISA. The polyclonal phage ELISA demonstrated significant enrichment of phages binding Api m 1 as well as the AM1-4 IgG/Api m 1 complex (FIG. 21A). Subsequently, monoclonal phages from the second round of selection were assessed and exhibited pronounced reactivity in phages ELISA (FIG. 21B). This suggested the presence of Api m 1 specific colonies binding Api m 1 at a distinct epitope than AM1-4.

Sequence analysis of all 19 positive clones identified 84% of the clones as the nanobody AM1-1. In addition to AM1-1, two new nanobodies A7 and B4 were identified. These nanobodies are highly divergent in the CDR sequences and are from now on referred to as nanobody AM1-21 and AM1-22, respectively (FIG. 22).

The two selected nanobodies were recloned into the expression vector pET22 b (+) and recombinantly expressed in bacteria. During the cloning, a pelB signal sequence, a polyhistidine-tag and a V5-tag was added to the nanobodies allowing for secretion into the periplasmic space and subsequent purification and detection. Thus, expressed nanobodies were purified from the culture supernatant by immobilized metal affinity chromatography, and the expected molecular mass of 15 kDa was confirmed by SDS-PAGE (FIG. 23).

### Conclusion

A nanobody phage library derived from an Api m 1 immunized llama was re-selected in order to identify nanobodies binding a distinct epitope than the nanobody AM1-4. In this selection the inventors selected nanobodies against immobilized AM1-4 IgG/Api m 1 complexes. This resulted in two new Api m 1 specific nanobodies.

### Example 15—Generation of Nanobody-Based Human IgG₁

### Aim of Study

In this study, the inventors aimed at generating nanobody-based human IgG₁ formats comprising the nanobodies AM1-21 and AM1-22.

### Materials and Methods

### Generation of Nanobody-Based Human IgG₁

Nanobody-based human IgG₁ (nb-hIgG₁) formats were generated by fusion of selected nanobodies to the human IgG₁ fc domain.

Nanobodies were amplified from the parental pCANTAB vectors by PCR and the product was cloned into a pCDNA3.1 vector between a human immunoglobulin-derived signal sequence and the human IgG₁ fc domains.

### Expression and Purification of Nanobody-Based Human IgG₁

Nb-hIgG₁ formats were expressed in mammalian FreeStyle 293-f cells. Transient transfection was performed with polyethylenimine (PEI) in a 1:2 ratio of DNA and PEI.

Expressed nb-hIgG₁ were purified from the supernatant of transient transfected cells by immobilized metal affinity chromatography on the ÄKTA™ start protein purification system using a 1 ml HisTrap™ Excel columns (GE Healthcare). The culture supernatants were run on the column and bound proteins were eluted with 500 mM Imidazole.

### Results

The Api m 1 specific nanobodies AM1-21 and AM1-22 were converted into homodimeric IgG₁ formats by fusion of the nanobodies to the human IgG₁ fc domains. The nanobody-based human IgG₁ (nb-hIgG₁) formats were expressed in mammalian cells and purified by immobilized metal affinity chromatography. The expressed nb-hIgG1 formats were analysed by SDS-Page (FIG. 24). The SDS-PAGE verified the expected molecular mass of approximately 80 kDa and suggested proper folding and dimerization of the nb-hIgG₁ format.

### Conclusion

The inventors successfully generated nanobody-based human IgG₁ formats comprising the nanobodies AM1-21 and AM1-22.

### Example 16—Characterization of Epitope Overlap

### Aim of Study

In this study, the inventors assessed the epitope overlap of the nanobodies AM1-1, AM1-4, AM1-21 and AM1-22. This will allow for identification of nanobodies that can be used in combination for blocking purposes.

### Materials and Methods

Epitope Binning with Nanobodies

Biolayer interferometry (BLI) measurements were performed at 30° C. on an Octet RED96 (ForteBio) using amine reactive second-generation (AR2G) biosensors (Sartorius). Sensors were prepared by dipping the sensors into a solution of nAPi m 1 at 20 μg/ml in 10 mM Na acetate pH 6.0 for 120s. The reaction was quenched for 300s in 1 M ethanolamine pH 8.5 and the baseline was obtained after 120s wash in kinetic buffer (PBS, 0.02% Tween20, 0.1% BSA). Association with nanobody was performed over 600s and the dissociation phase in the same buffer was monitored for 120s. The secondary association of nanobody to the antigen was assessed in the same manner.

Epitope Binning with Nanobody-Based IgG₁

BLI measurements were performed at 30° C. on an Octet RED96 (ForteBio) using Anti-hIgG Fc Capture (AHC) biosensors (Sartorius). Sensors were loaded by dipping the sensors into a solution of 50 nM nanobody-based human IgG₁ in kinetic buffer (PBS, 0.02% Tween20, 0.1% BSA) for 600s. The sensors were blocked with 50 μg/ml non-specific nanobody-based human IgG₁ for 600s and the baseline was obtained after 240s wash in kinetic buffer. Association with nApi m 1 was performed over 600s and the dissociation phase in the same buffer was monitored for 180s. The secondary association was performed with 50 nM nanobody-based human IgG₁ for 600s.

### Results

In order to identify nanobodies binding non-overlapping epitopes the inventors analyzed sequential binding of nanobodies to immobilized Api m 1 using biolayer interferometry. The binding studies confirmed that the nanobodies AM1-21 and AM1-22 bind epitopes that do not overlap with the nanobody AM1-4. In addition, the sequential binding studies indicated that the nanobody AM1-21 binds an epitope that does not overlap with neither AM1-1 nor AM1-22. In contrast, AM1-22 binds an epitope overlapping with AM1-1 (FIG. 25).

In order to analyze whether or not the nanobody-based IgG₁ formats can be used in combinations, the inventors assessed the sequential binding of the nb-IgG₁ formats to Api m 1 using biolayer interferometry. The binding studies confirmed that the nanobody-based human IgG₁ formats AM1-21 IgG₁ and AM1-22 IgG₁ can bind at the same time as AM1-4 IgG₁. However, AM11-21 IgG₁, AM-22 IgG₁ and AM1-1 IgG₁ cannot bind at the same time indicating that they bind epitopes at least in close proximity and that the sequential binding observed with the nanobodies is sterically blocked by the larger IgG formats (FIG. 26).

### Conclusion

The binding studies show that the nanobody AM1-21 binds an epitope that does not overlap with AM1-1 nor AM1-22. However, the nanobody-based IgG₁ formats cannot bind at the same time suggesting steric hindrance by the chosen larger format.

### Example 17—Kinetics of AM1-21 and AM1-22 IgG₁

### Aim of Study

In this study, the inventors aimed at determining the affinity of the nanobody-based IgG₁ formats AM1-21 and AM1-22 IgG₁.

### Material and Methods

### Affinity Determination Using Biolayer Interferometry

Biolayer interferometry (BLI) measurements were performed at 30° C. on an Octet RED96 system (ForteBio) using Anti-Human Fc Capture (AHC) Biosensors (Sartorius). All samples were prepared in Kinetic buffer (PBS pH 7.4, 0.1% BSA and 0.02% Tween20). Sensors were loaded with 2 nM nanobody-based human IgG₁ for 420s and a baseline was obtained after 300s in Kinetic buffer. Association was performed over 300s with 2-fold dilution series of nApi m 1 from 60 nm to 3.75 nM. Dissociation was measured for 1800s in Kinetic buffer. Binding in the absence of an allergen was used as a reference to subtract the non-specific interactions with the biosensor. Curve fitting was performed with Octet software using 1:1 binding model.

### Results

In this study the inventors analyzed the affinity of AM1-21 IgG and AM1-22 IgG using biolayer interferometry (FIG. 27). The two nanobody-based hIgG₁ AM1-21 IgG and AM1-22 IgG bind nApi m 1 with high affinities of 1.53·10⁻¹¹ M and below detection level (1.0·10⁻¹² M) respectively.

### Conclusion

Using biolayer interferometry the inventors showed that the nanobody-based human IgG₁ formats AM1-21 IgG₁ and AM1-22 IgG₁ bind Api m 1 with affinities in the picomolar range. The affinities of AM1-21 IgG₁ and AM1-22 IgG₁ are approximately 100-fold higher than that of the AM1-1 IgG₁. Obtaining nanobodies of picomolar affinity via the targeted selection strategy described here is surprising. The nanobodies with strongly increased affinities and the epitopes in proximity or similar to the one of AM1-1 could potentially provide an advantage when applied for blocking purposes.

### REFERENCES

Griffiths, A. D., et al.: Isolation of high affinity human antibodies directly from large synthetic repertoires. EMBO J, 1994. 13(14): p. 3245-60.

TABLE 3
Sequence listing
Sequences of Nanobodies targeting
Api m 1, including CDRs.

| SEQ | | |
|---|---|---|
| ID | Name | Amino acid sequence |
| 1 | AM1-1 | QVQLVETGGGLVQAGGSLRLSCATS-GTIFSRATMA- |
| | | WYRQTPGKQREWVT-TITTSGNTN- |
| | | YADSVKGRFTISRDNAESTLYLQMNSLKPEDTAVYYCN- |
| | | AQFLSSRTNY-WGKGTQVTVS |
| 2 | AM1-2 | QVQLVESGGGLVQAGGSLRLSCAAS-RRTFSGYAMG- |
| | | WFRQAPGKEREFVA-AHWGSTSTH- |
| | | YADSVKGRFTISRDNAKNTVYLQMNTLKLEDTAVYYCA |
| | | AKDRLGAVSDDQAYDY-WGQGTQVTVS |
| 3 | AM1-3 | QVQLVESGGGLVQAGGSLRLSCAAS-GRAFSSYPMG- |
| | | WFRQAPGKEREFLA-AISGSGRLTY- |
| | | YADSVEGRFTISRDNAKNTVYLQMDVLTPEDTAIYYCA- |
| | | AQKPYASAQRWPPENYDF-WGQGTQVTVS |
| 4 | AM1-4 | QVQLVESGGGLVQAGGSLRLSCAAS-GRTFSRYAMG- |
| | | WFRQAPGKEREFVS-AISGSGGFTD- |
| | | YADSVKGRFTISRDNAKSTVYLRMSSLKPEDTAVYYCA- |
| | | AEGSRGSSTRLDARGTYDY-WGQGTQVTVS |
| 5 | AM1-5 | QVQLVESGGGLVQAGGSLRLSCAAS-GRAFSSRPIG- |
| | | WFRQAPAKEREFVG-VISQSGGFTD- |
| | | YEDSVKGRFTISRDNAKNTVYLQMSSLQPEDTAVYYCA- |
| | | TGILFASGSAYYRPDDYDY-WGQGTQVTVS |
| 6 | AM1-6 | QVQLVETGGGLVQAGGSLRLSCAFS-GRTFGNYAMA- |
| | | WFRQAPGKERDFV-AYISWSGSSVF- |
| | | HADSVKGRFTISRDNAKNTVYLQMSSLKAEDTAVYYCA- |
| | | AAERTKRVSITDDTSYDY-WGQGTQVTVS |
| 7 | AM1-7 | QVQLVETGGGLVQAGGSLRLSCAAS-GRTFSSYEMA- |
| | | WFRQAPGKEREFVA-VLSWTGRSTY- |
| | | YADSVKGRFTISRDNAKNTVYLQMNNLRPEDTAVYYCA- |
| | | ADPPRHGYTWPPDGMDY-WGKGTQVTVS |
| 8 | AM1-8 | QVQLVESGGGFVQAGGSLRLSCVSS-GGIFAIGKLA- |
| | | WYRQAPGQMREFVA-SISSGKIVSY- |
| | | ESHVKGRFTISRDNTKDTVYLQMDSLKPEDTGVYYCA- |
| | | IQAPENWATTEYDY-WGQGTQVTVS |
| 9 | AM1-9 | QVQLVESGGGLVQAGGSLRLSCAAS-GRTGSSTTYRMA- |
| | | WFRQAPGKEREFVA-HGTCNGGTY- |
| | | YADSVKGRFTISRNNAKNTVYLQMNNLKAEDTAVYYCA- |
| | | AARPTGASGRSVYDLDY-WGQGTQVTVS |
| 10 | AM1-10 | QVQLVETGGGLVQAGGSLRLSCAAS-GRTASDYVIA |
| | | WFRQAPGKVRVFVA-GISSGGTIKD- |
| | | YAESESVKGRFTISRDNAKNTVYLQMNSLKPEDTAVYYC |
| | | A-ARLRVTDFDAASYDY-WGQGTQVTVS |
| 11 | AM1-2.2 | QVQLVESGGGLVQAGGSLRLSCAAS-SRTFSGYAVG- |
| | | WFRQAPRKEREFVA-AFWGRTSTH- |
| | | YADSVKGRFTISRDNVKNTVYLQMNSLNPEDTAVYYCA- |
| | | AKDRLGAVSDDHAYDY-WGQGTQVTVS |
| 12 | AM1-7.2 | QVQLVESGGGLVQAGGSLRLSCAAS-GRTVSSYDMA- |
| | | WFRQAPGKERDFVA-VISWTGRATY- |
| | | YEDSVKGRFTISRDDAKNTVYLQMNNLRPEDTAVYYCA- |
| | | ADPPRHGASWPPSGMDY-WGKGTQVTVS |

Bold: CDR1; Underlining: CDR2; Italic: CDR3

TABLE 4
CDRs for the Nanobodies targeting
Api m 1 according to table 3.

| SEQ ID | Name | Amino acid sequence |
|---|---|---|
| 13 | AM1-1-CDR1 | GTIFSRATMA |
| 14 | AM1-1-CDR2 | TITTSGNTN |
| 15 | AM1-1-CDR3 | AQFLSSRTNY |
| 16 | AM1-2-CDR1 | RRTFSGYAMG |
| 17 | AM1-2-CDR2 | AHWGSTSTH |
| 18 | AM1-2-CDR3 | AKDRLGAVSDDQAYDY |
| 19 | AM1-3-CDR1 | GRAFSSYPMG |
| 20 | AM1-3-CDR2 | AISGSGRLTY |
| 21 | AM1-3-CDR3 | AQKPYASAQRWPPENYDF |
| 22 | AM1-4-CDR1 | GRTFSRYAMG |
| 23 | AM1-4-CDR2 | AISGSGGFTD |
| 24 | AM1-4-CDR3 | AEGSRGSSTRLDARGTYDY |
| 25 | AM1-5-CDR1 | GRAFSSRPIG |
| 26 | AM1-5-CDR2 | VISQSGGFTD |
| 27 | AM1-5-CDR3 | TGILFASGSAYYRPDDYDY |
| 28 | AM1-6-CDR1 | GRTFGNYAMA |
| 29 | AM1-6-CDR2 | AYISWSGSSVF |
| 30 | AM1-6-CDR3 | AAERTKRVSITDDTSYDY |
| 31 | AM1-7-CDR1 | GRTFSSYEMA |
| 32 | AM1-7-CDR2 | VLSWTGRSTY |
| 33 | AM1-7-CDR3 | ADPPRHGYTWPPDGMDY |
| 34 | AM1-8-CDR1 | GGIFAIGKLA |
| 35 | AM1-8-CDR2 | SISSGKIVSY |
| 36 | AM1-8-CDR3 | IQAPENWATTEYDY |
| 37 | AM1-9-CDR1 | GRTGSSTTYRMA |
| 38 | AM1-9-CDR2 | HGTCNGGTY |
| 39 | AM1-9-CDR3 | AARPTGASGRSVYDLDY |
| 40 | AM1-10-CDR1 | GRTASDYVIA |
| 41 | AM1-10-CDR2 | GISSGGTIKD |
| 42 | AM1-10-CDR3 | ARLRVTDFDAASYDY |
| 43 | AM1-2.2-CDR1 | SRTFSGYAVG |
| 44 | AM1-2.2-CDR2 | AFWGRTSTH |
| 45 | AM1-2.2-CDR3 | AKDRLGAVSDDHAYDY |
| 46 | AM1-7.2-CDR1 | GRTVSSYDMA |
| 47 | AM1-7.2-CDR2 | VISWTGRATY |
| 48 | AM1-7.2-CDR3 | ADPPRHGASWPPSGMDY |

TABLE 5
Sequences of Nanobodies targeting
Api m 2, including CDRs.

| SEQ | | |
|---|---|---|
| ID | Name | Amino acid sequence |
| 49 | AM2-A1 | QVQLQESGGGLVPPGGSLRLSCAAS-GGPLDHYTIA- |
| | | WFRQAPGKEREVVS-CIGSSDVSTY- |
| | | YADSVKGRFAISRDNVKNTVFLQMSSLKPEDTAVYLCA- |
| | | AKYVGSTSWGGGCAYELSVDRYLEV-WGQGTQVSVS |
| 50 | AM2-A2 | QVQLQESGGGLVQAGGSLNLSCATS-ATFRVRLGPMS- |
| | | WYRQAPGKQRELVA-SITSGGSTN- |
| | | YSDSVRGRFTISRDNAKNAVYPQMNSLQTEDTAVYCCR- |
| | | ADQDLPPGLIGTGAVV-WGQGTQVAVS |
| 51 | AM2-C2 | EVQLQESGGGLVQPGGSLRLSCAAS-GTTINIGAAR- |
| | | WYRQAPGKQREVVA-GISFGGATN- |
| | | YIDSVKGRFTISRGDALNTWYLQMNNLKPEDTAVYYCN- |
| | | ARGE-WGQGTQVAVS |
| 52 | AM2-J1 | QVQLQESGGGLVQAGGSLRLSCVAS-GSGSSNNQVN- |
| | | WYRQAPGKQRELIA-RISTGGTTN- |
| | | YIDAVKGRFTIARDNAERSVYLQMNNLGPEDTAIYYCH- |
| | | ATIRARSY-WGQGTLVTVS |
| 53 | AM2-O2 | EVQLQESGGGLVQAGGSLRLSCAAS-GDTICISAMG- |
| | | WYRQAPGKERELVA-VITSADSTN- |
| | | YADSVKGRFTISRAGAKNTLYLQMNSLKPEDTAVYYCN- |
| | | AHTPKYSDFEVTCYEYDY-WGQGTLVSVS |

Bold: CDR1; Underlining: CDR2; Italic: CDR3

TABLE 6
CDRs for the Nanobodies targeting
Api m 2 according to Table 5.

| SEQ ID | Name | Amino acid sequence |
|---|---|---|
| 54 | AM2-A1-CDR1 | GGPLDHYTIA |
| 55 | AM2-A1-CDR2 | CIGSSDVSTY |
| 56 | AM2-A1-CDR3 | AKYVGSTSWGGGCAYELSVDRYLEV |
| 57 | AM2-A2-CDR1 | ATFRVRLGPMS |
| 58 | AM2-A2-CDR2 | SITSGGSTN |
| 59 | AM2-A2-CDR3 | ADQDLPPGLIGTGAVV |
| 60 | AM2-C2-CDR1 | GTTINIGAAR |
| 61 | AM2-C2-CDR2 | GISFGGATN |
| 62 | AM2-C2-CDR3 | ARGE |
| 63 | AM2-J1-CDR1 | GSGSSNNQVN |
| 64 | AM2-J1-CDR2 | RISTGGTTN |
| 65 | AM2-J1-CDR3 | ATIRARSY |
| 66 | AM2-O2-CDR1 | GDTICISAMG |
| 67 | AM2-O2-CDR2 | VITSADSTN |
| 68 | AM2-O2-CDR3 | AHTPKYSDFEVTCYEYDY |

SEQ ID NO: 69
>sp|P00630|PA2_APIME Phospholipase A2 OS =
Apis mellifera OX = 7460 PE = 1 SV = 3
MQVVLGSLFLLLLSTSHGWQIRDRIGDNELEER IIYPGTL
WCGHGNKSSGPNELGRFKHTDACCRTH DMCPDVMSAG
ESKHGLTNTASHTRLSCDCDDKFYDCLKNSADTISSYFV
GKMYFNLIDTKCYKLEHPVTGCGERTEGRCLHYTVDKSKP
KVYQWFDLRKY
SEQ ID NO: 70
Active protein part of PLA2 (SEQ ID NO: 69)
(without signal sequence and propeptide).
H34, D64 and Y87 are highlighted in bold.
IIYPGTLWCGHGNKSSGPNELGRFKHTDACCRTHDMCPDVMSAG
ESKHGLTNTASHTRLSCDCDDKFYDCLKNSADTISSYFVGKMYFN
LIDTKCYKLEHPVTGCGERTEGRCLHYTVDKSKPKVYQWFDLRKY

TABLE 7
Sequences of Nanobodies AM1-21 and AM1-22
targeting Api m 1, including CDRs.

| SEQ | | |
|---|---|---|
| ID | Name | Amino acid sequence |
| 71 | AM1-21 | QVQLVESGGGLVQAGGSLRLSCAAS-GFTLDYYAIG- |
| | | WFRQAPGKEREGVS-CITSYDDSTY- |
| | | YTDSVKGRFTISRDNAKNTVYLQMNSLKPEDTAVYYCA- |
| | | AVGTWYLVNWWAPSEYDY-WGQGTQVTVS |
| 72 | AM1-22 | QVQLVESGGGLVQAGGSLRLSCAAS-GSIFNWA- |
| | | WYRQFPGKQREWVA-TITSGGNTN- |
| | | LADSVKGRFTISLDNAKNTMYLQMNSLKPEDTAVYYCN- |
| | | SARFRNRDAI-WGQGTQVTVS |

Bold: CDR1; Underlining: CDR2; Italic: CDR3

TABLE 8
CDRs for the Nanobodies AM1-21 and AM1-22
targeting Api m 1 according to Table 7.

| SEQ ID | Name | Amino acid sequence |
|---|---|---|
| 73 | AM1-21-CDR1 | GFTLDYYAIG |
| 74 | AM1-21-CDR2 | CITSYDDSTY |
| 75 | AM1-21-CDR3 | AVGTWYLVNWWAPSEYDY |
| 76 | AM1-22-CDR1 | GSIFNWA |
| 77 | AM1-22-CDR2 | TITSGGNTN |
| 78 | AM1-22-CDR3 | SARFRNRDAI |

TABLE 9
DNA Sequences encoding Nanobodies AM1-21 and
AM1-22 targeting Api m 1.

| | |
|---|---|
| AM1-21 | CAGGTGCAGCTCGTGGAGTCAGGGGGAGGCCTGGTGCAGGCT |
| SEQ ID | GGGGGGTCTCTGAGACTCTCCTGTGCAGCCTCTGGATTCACT |
| NO: 79 | TTGGATTATTATGCCATAGGCTGGTTCCGCCAGGCCCCAGGG |
| | AAGGAGCGCGAGGGGGTCTCATGTATTACTAGTTATGATGAT |
| | AGCACATACTATACAGACTCCGTGAAGGGCCGATTCACCATC |
| | TCCAGAGACAATGCCAAGAACACGGTGTATCTGCAAATGAAC |
| | AGCCTGAAACCTGAGGACACGGCCGTTTATTACTGTGCCGCA |
| | GTGGGTACCTGGTATCTCGTAAACTGGTGGGCCCCATCTGAG |
| | TATGACTACTGGGGCCAGGGGACCCAGGTCACCGTCTCC |
| AM1-22 | CAGGTGCAGCTCGTGGAGTCGGGGGGAGGCTTGGTGCAGGCT |
| SEQ ID | GGGGGGTCTCTGAGACTCTCCTGTGCAGCCTCTGGAAGCATC |
| NO: 80 | TTCAATTGGGCCTGGTACCGCCAGTTTCCAGGGAAACAGCGC |
| | GAGTGGGTTGCGACGATTACTAGTGGTGGTAACACAAACTTG |
| | GCAGACTCCGTGAAGGGCCGATTCACCATCTCCTTGGACAAC |
| | GCCAAGAACACGATGTATCTGCAGATGAACAGCCTGAAACCT |
| | GAAGACACAGCCGTCTATTATTGTAATTCCGCCCGGTTCCGC |
| | AACAGAGATGCCATCTGGGGCCAGGGGACCCAGGTCACCGTC |
| | TCC |

1. A single domain antibody having binding affinity for Api m 1, wherein said single domain antibody comprises CDR domains selected from the group consisting of:
i. CDR domains:
CDR1 according to SEQ ID NO: 13;
CDR2 according to SEQ ID NO: 14; and
CDR3 according to SEQ ID NO: 15;
ii. CDR domains:
CDR1 according to SEQ ID NO: 16;
CDR2 according to SEQ ID NO: 17; and
CDR3 according to SEQ ID NO: 18;
iii. CDR domains:
CDR1 according to SEQ ID NO: 19;
CDR2 according to SEQ ID NO: 20; and
CDR3 according to SEQ ID NO: 21;
iv. CDR domains:
CDR1 according to SEQ ID NO: 22;
CDR2 according to SEQ ID NO: 23; and
CDR3 according to SEQ ID NO: 24;
v. CDR domains:
CDR1 according to SEQ ID NO: 25;
CDR2 according to SEQ ID NO: 26; and
CDR3 according to SEQ ID NO: 27;
vi. CDR domains:
CDR1 according to SEQ ID NO: 28;
CDR2 according to SEQ ID NO: 29; and
CDR3 according to SEQ ID NO: 30;
vii. CDR domains:
CDR1 according to SEQ ID NO: 31;
CDR2 according to SEQ ID NO: 32; and
CDR3 according to SEQ ID NO: 33.
viii. CDR domains:
CDR1 according to SEQ ID NO: 34;
CDR2 according to SEQ ID NO: 35; and
CDR3 according to SEQ ID NO: 36;
ix. CDR domains:
CDR1 according to SEQ ID NO: 37;
CDR2 according to SEQ ID NO: 38; and
CDR3 according to SEQ ID NO: 39;
x. CDR domains:
CDR1 according to SEQ ID NO: 40;
CDR2 according to SEQ ID NO: 41; and
CDR3 according to SEQ ID NO: 42;
xi. CDR domains:
CDR1 according to SEQ ID NO: 43;
CDR2 according to SEQ ID NO: 44; and
CDR3 according to SEQ ID NO: 45;
xii. CDR domains:
CDR1 according to SEQ ID NO: 46;
CDR2 according to SEQ ID NO: 47; and
CDR3 according to SEQ ID NO: 48;
xiii. CDR domains:
CDR1 according to SEQ ID NO: 73;
CDR2 according to SEQ ID NO: 74; and
CDR3 according to SEQ ID NO: 75; and
xiv. CDR domains:
CDR1 according to SEQ ID NO: 76;
CDR2 according to SEQ ID NO: 77; and
CDR3 according to SEQ ID NO: 78;
or single domain antibodies according to any of i.-xiv. having 1-3 substitutions in one or more of the CDR1, CDR2 and CDR3 domains, preferably 1-2 substitutions and more preferably 1 substitution in one or more of the CDR1, CDR2 and CDR3 domains; OR
a single domain antibody having binding affinity for Api m 2, wherein said single domain antibody comprises CDR domains selected from the group consisting of:
xv. CDR domains:
CDR1 according to SEQ ID NO: 54;
CDR2 according to SEQ ID NO: 55; and
CDR3 according to SEQ ID NO: 56;
xvi. CDR domains:
CDR1 according to SEQ ID NO: 57;
CDR2 according to SEQ ID NO: 58; and
CDR3 according to SEQ ID NO: 59;
xvii. CDR domains:
CDR1 according to SEQ ID NO: 60;
CDR2 according to SEQ ID NO: 61; and
CDR3 according to SEQ ID NO: 62;
xviii. CDR domains:
CDR1 according to SEQ ID NO: 63;
CDR2 according to SEQ ID NO: 64; and
CDR3 according to SEQ ID NO: 65; and
xix. CDR domains:
CDR1 according to SEQ ID NO: 66;
CDR2 according to SEQ ID NO: 67; and
CDR3 according to SEQ ID NO: 68;
or single domain antibodies according to any of xv.-xix. having 1-3 substitutions in one or more of the CDR1, CDR2 and CDR3 domains, preferably 1-2 substitutions and more preferably 1 substitution in one or more of the CDR1, CDR2 and CDR3 domains.

2. A single domain antibody according to claim 1, wherein said single domain antibody comprises CDR domains selected from the group consisting of:
i. CDR domains:
CDR1 according to SEQ ID NO: 13;
CDR2 according to SEQ ID NO: 14; and
CDR3 according to SEQ ID NO: 15;
ii. CDR domains:
CDR1 according to SEQ ID NO: 16;
CDR2 according to SEQ ID NO: 17; and
CDR3 according to SEQ ID NO: 18;
iii. CDR domains:
CDR1 according to SEQ ID NO: 19;
CDR2 according to SEQ ID NO: 20; and
CDR3 according to SEQ ID NO: 21;
iv. CDR domains:
CDR1 according to SEQ ID NO: 22;
CDR2 according to SEQ ID NO: 23; and
CDR3 according to SEQ ID NO: 24;
v. CDR domains:
CDR1 according to SEQ ID NO: 25;
CDR2 according to SEQ ID NO: 26; and
CDR3 according to SEQ ID NO: 27;
vi. CDR domains:
CDR1 according to SEQ ID NO: 28;
CDR2 according to SEQ ID NO: 29; and
CDR3 according to SEQ ID NO: 30;
vii. CDR domains:
CDR1 according to SEQ ID NO: 31;
CDR2 according to SEQ ID NO: 32; and
CDR3 according to SEQ ID NO: 33.
viii. CDR domains:
CDR1 according to SEQ ID NO: 34;
CDR2 according to SEQ ID NO: 35; and
CDR3 according to SEQ ID NO: 36;
ix. CDR domains:
CDR1 according to SEQ ID NO: 37;
CDR2 according to SEQ ID NO: 38; and
CDR3 according to SEQ ID NO: 39;
x. CDR domains:
CDR1 according to SEQ ID NO: 40;
CDR2 according to SEQ ID NO: 41; and
CDR3 according to SEQ ID NO: 42;
xi. CDR domains:
CDR1 according to SEQ ID NO: 43;
CDR2 according to SEQ ID NO: 44; and
CDR3 according to SEQ ID NO: 45;
xii. CDR domains:
CDR1 according to SEQ ID NO: 46;
CDR2 according to SEQ ID NO: 47; and
CDR3 according to SEQ ID NO: 48;
xiii. CDR domains:
CDR1 according to SEQ ID NO: 73;
CDR2 according to SEQ ID NO: 74; and
CDR3 according to SEQ ID NO: 75; and
xiv. CDR domains:
CDR1 according to SEQ ID NO: 76;
CDR2 according to SEQ ID NO: 77; and
CDR3 according to SEQ ID NO: 78; OR
a single domain antibody having binding affinity for Api m 2, wherein said single domain antibody comprises CDR domains selected from the group consisting of:
xv. CDR domains:
CDR1 according to SEQ ID NO: 54;
CDR2 according to SEQ ID NO: 55; and
CDR3 according to SEQ ID NO: 56;
xvi. CDR domains:
CDR1 according to SEQ ID NO: 57;
CDR2 according to SEQ ID NO: 58; and
CDR3 according to SEQ ID NO: 59;
xvii. CDR domains:
CDR1 according to SEQ ID NO: 60;
CDR2 according to SEQ ID NO: 61; and
CDR3 according to SEQ ID NO: 62;
xviii. CDR domains:
CDR1 according to SEQ ID NO: 63;
CDR2 according to SEQ ID NO: 64; and
CDR3 according to SEQ ID NO: 65;
xix. CDR domains:
CDR1 according to SEQ ID NO: 66;
CDR2 according to SEQ ID NO: 67; and
CDR3 according to SEQ ID NO: 68.

3. The single domain antibody according to claim 1, being selected from the group consisting of
a) SEQ ID NO's: 1-12, SEQ ID NO's: 71-72, and SEQ ID NO's: 49-53; or
b) a single domain antibody having at least 70%, such at least 80%, for example at least 85%, such as at least 90% or more than 95% sequence identity, such as 100% sequence identity with the amino acid according to a), with the proviso that said single domain antibody comprises a CDR1 domain, a CDR2 domain and a CDR3 domain according to any of i.-xiv., or xv.-xix. as defined in claim 1.

4. (canceled)

5. The single domain antibody according to claim 1, comprising the CDR domains
CDR1 according to SEQ ID NO: 13;
CDR2 according to SEQ ID NO: 14; and
CDR3 according to SEQ ID NO: 15.

6. The single domain antibody according to claim 1, comprising SEQ ID NO: 1.

7. The single domain antibody according claim 1, being coupled to an Ig, such as IgE or IgG, such as IgG4 or IgG1, preferably to the Fc part of the Ig, such as the Fc part of IgG or the Fc part of IgE.

8. The single domain antibody according to claim 1, wherein the single domain antibody is comprised in a multi-specific antibody.

9. The single domain antibody according to claim 8, wherein the multi-specific antibody comprises one or more of the single domain antibodies selected from the group consisting of:
a. a single domain antibody having the CDR domains:
i. CDR1 sequence according to SEQ ID NO: 13;
ii. CDR2 sequence according to SEQ ID NO: 14; and
iii. CDR3 sequence according to SEQ ID NO: 15;
b. a single domain antibody having the CDR domains:
i. CDR1 according to SEQ ID NO: 73;
ii. CDR2 according to SEQ ID NO: 74; and
iii. CDR3 according to SEQ ID NO: 75; and/or
c. a single domain antibody having the CDR domains:
i. CDR1 according to SEQ ID NO: 76;
ii. CDR2 according to SEQ ID NO: 77; and
iii. CDR3 according to SEQ ID NO: 78.

10. (canceled)

11. (canceled)

12. The single domain antibody according to claim 1, having binding affinity for the active site of Api m 1, wherein the active site is any of the residues H34, D64 and Y87 of SEQ ID NO: 70.

13. (canceled)

14. The single domain antibody according to claim 1, wherein the single domain antibody or multi-specific antibody binds to one or more of the residues Cys31, His34, Asp35, Ala43, Glu45, Ser46, and Tyr87 of SEQ ID NO: 70.

15. The single domain antibody according to claim 1, wherein the single domain antibody is comprised in a bi-specific antibody.

16. The single domain antibody according to claim 1, wherein the catalytic activity of Api m 1 is reduced after being contacted with the single domain antibody or multi-specific antibody.

17. The single domain antibody according to claim 1, wherein the single domain antibody is comprised in a composition or combination.

18. (canceled)

19. (canceled)

20. (canceled)

21. (canceled)

22. A composition comprising:
a) a nucleic acid encoding a single domain antibody according to claim 1; and/or
b) the nucleic acid according to a), comprised in a vector; and/or
c) the nucleic acid according to a) or the vector according to b), comprised in a host cell.

23. The single domain antibody according to claim 1 for in vitro use, wherein the in vitro use is standardization, such as for standardizations of diagnostic or therapeutic products.

24. A method for treatment, prevention and/or alleviation of bee allergy, or a venom induced toxicity in a subject in need thereof, the method comprising administering one or more of the single domain antibodies according to claim 1 or a single domain antibody binding to one or more one or more of the residues Cys31, His34, Asp35, Ala43, Glu45, Ser46, Asp64 and Tyr87 of SEQ ID NO: 70.
